(19) United States
(12) Patent Application Publication
(10) Pub. No.: US 2020/0187500 A1
(43) Pub. Date: Jun. 18, 2020

(54) PYRIDINE COMPOUNDS

(71) Applicant: BASF SE, Ludwigshafen am Rhein (DE)

(72) Inventors: Ana ESCRIBANO CUESTA, Ludwigshafen (DE); Bernd MUELLER, Ludwigshafen (DE); Erica CAMBEIS, Sale (GB); Antje WOLF, Ludwigshafen (DE); Nadine RIEDIGER, Limburgerhof (DE); Marcus FEHR, Limburgerhof (DE); Jan Klaas LOHMANN, Ludwigshafen (DE); Michael SEET, Ludwigshafen (DE); Wassilios GRAMMENOS, Ludwigshafen (DE); Christian Harald WINTER, Ludwigshafen (DE); Violeta TERTERYAN-SEISER, Limburgerhof (DE); Thomas GROTE, Ludwigshafen (DE)

(21) Appl. No.: 16/500,541

(22) PCT Filed: Mar. 26, 2018
(86) PCT No.: PCT/EP2018/057594

§ 371 Date: Oct. 3, 2019

(30) Foreign Application Priority Data
Apr. 6, 2017 (EP) 17165179.7

(51) Int. Cl. A01N 43/42; C07D 401/04; A01M 21/04; A01C 1/06

(52) U.S. Cl. CPC A01N 43/42; A01C 1/06; A01M 21/043; C07D 401/04

(57) ABSTRACT

The present invention relates to compounds of formula I

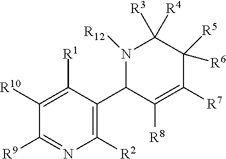

wherein the variables are defined as given in the description and claims. The invention further relates to uses and composition for compounds of formula I.

The present invention relates to pyridine compounds and the N-oxides and the salts thereof for combating phytopathogenic fungi, and to the use and methods for combating phytopathogenic fungi and to seeds coated with at least one such compound. The invention also relates to processes for preparing these compounds, intermediates, processes for preparing such intermediates, and to compositions comprising at least one compound I.

In many cases, in particular at low application rates, the fungicidal activity of the known fungicidal compounds is unsatisfactory. Based on this, it was an object of the present invention to provide compounds having improved activity and/or a broader activity spectrum against phytopathogenic harmful fungi.

Surprisingly, this object is achieved by the use of the inventive pyridine compounds of formula I having favorable fungicidal activity against phytopathogenic fungi.

Accordingly, the present invention relates to the compounds of formula I

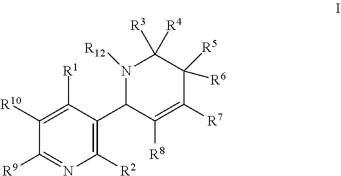

wherein
R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
   R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
   wherein the aliphatic moieties of R¹ are unsubstituted or substituted with identical or different groups R^{1a} which independently of one another are selected from:
   R^{1a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
   wherein the cycloalkyl, heteroaryl and aryl moieties of R¹ are not further substituted or carry 1, 2, 3, 4, 5 or up to the maximum number of identical or different groups R^{1b} which independently of one another are selected from:
   R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
   R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
   wherein the aliphatic moieties of R² are unsubstituted or substituted with identical or different groups R^{2a} which independently of one another are selected from:
   R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
   wherein the cycloalkyl, heteroaryl and aryl moieties of R¹ are not further substituted or carry 1, 2, 3, 4, 5 or up to the maximum number of identical or different groups R^{2b} which independently of one another are selected from:
   R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
   wherein the aliphatic moieties of and R⁴ are independently not further substituted or carry 1, 2, 3 or up to the maximum possible number of identical or different groups R^{4a}, respectively, which independently of one another are selected from:
   R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
   R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
   and wherein R^{x} is as defined above; or
   n is 0, 1, 2 or
R³, R⁴ together with the carbon atom to which they are bound (marked with * in formula I) form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
   o is 0, 1, 2 or 3; and
   R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)ₙ—C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
      and
      wherein the aliphatic moieties of R⁷⁸ are not further substituted or carry 1, 2, 3 or up to the maximum possible number of identical or different groups R^{78a} which independently of one another are selected from:
      R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
      wherein the alicyclic, phenyl, heterocyclic and heteroaryl moieties of R⁷⁸ are unsubstituted or substituted with identical or different groups R^{78b} which independently of one another are selected from:
      R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
   R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
   wherein the acyclic moieties of R⁹ are unsubstituted or substituted by groups R^{9a} which independently of one another are selected from:
   R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
   wherein the carbocyclic, heteroaryl and aryl moieties of R⁹ are unsubstituted or substituted by groups R^{9b} which independently of one another are selected from:
   R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
   and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent RN selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   R^{x} is as defined above;
      wherein the acyclic moieties of R¹¹ are unsubstituted or carry 1, 2, 3 or up to the maximum possible number of identical or different groups R^{11a} which independently of one another are selected from:
   R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{11a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
      wherein the carbocyclic, heterocyclic, heteroaryl and aryl moieties of R¹¹ are unsubstituted or substituted with identical or different groups R^{11b} which independently of one another are selected from:
   R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
   R^{Y} is defined as above;
   wherein the acyclic moieties of R¹² are not further substituted or carry one, two, three or up to the maximum possible number of identical or different groups R^{12a} which independently of one another are selected from:
   R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
      wherein the alicyclic, phenyl, heterocyclic and heteroaryl moieties of R¹² are not further substituted or carry one, two, three, four, five or up to the maximum number of identical or different groups R^{12b} which independently of one another are selected from:
   R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
   and wherein n is defined as above;
      with the proviso that if R⁷, R⁸ together with the carbon atoms to which they are bound form a ring A, wherein the ring A is phenyl
R¹ is hydrogen; and
R⁴ cannot be an unsubstituted C₁-C₆-alkyl;
   and the N-oxides and the agriculturally acceptable salts thereof.

The numbering of the ring members in the compounds of the present invention is as given in formula I above:

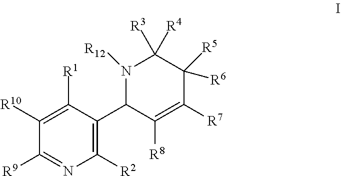

Compounds of formula I, when R¹² is not proton, can be accessed e.g. starting from compounds of the formula I-1 (R¹² is proton) A skilled person will realize that compounds of type I can be reached via reaction with a reactive group R¹²—W. Reactive groups are preferably C₁-C₈-alkyl halides, C₂-C₆-alkenyl halides, C₂-C₆-alkynyl halides, benzyl halides, aldehydes, ester, acid chlorides, amides, sulfates, silyl halides or phosphates, e.g. carboxylic acid (W═OH), aldehydes (W═H), acid chloride (W═Cl), amides (W═NMe₂) or phosphates (W═OCH₃).

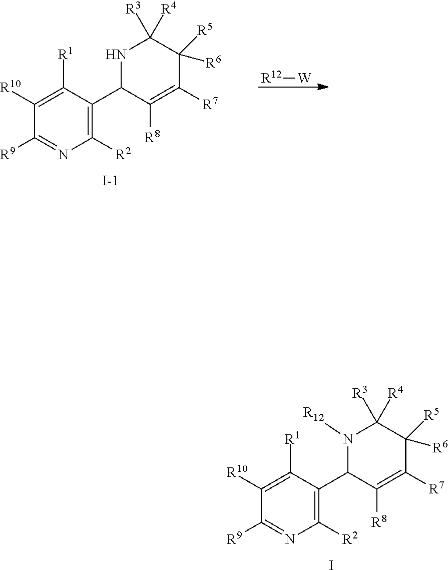

Typically the reaction is performed in a range between 0° C. and ambient temperature in the presence of a reactive group and organic base. Suitable base preferably NEt₃, pyridine NaOH, TEBAC, K₂CO₃, NaCO₃ or KOH. Most preferably solvents are THF, DMF, DMSO, MeOH or water (see for example, Journal of Medicinal Chemistry, 1989, 32(6), 1242-1248; European Journal of Medicinal Chemistry, 2009, 44(10), 4034-4043).

Compounds of formula I-1 can be accessed e.g. starting from compounds of the formula II via a reduction agent in an organic solvent (see for example WO2009095253, WO2008143263). Reduction agent can be for example NaBH₄ or NaCNBH₃.Typically the reaction is performed in a range between 0° C., room temperature and 60° C. in an organic solvent, such as THF, dichloromethane, acetonitrile, MeOH, EtOH or water or in a mixture of organic solvent and water.

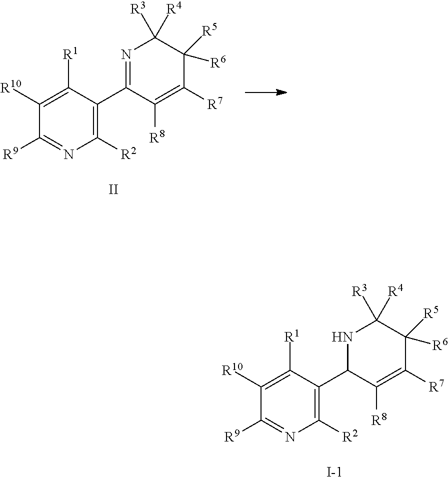

Compounds of formula II can be also reduced to I-1 via hydrogenation by using a metal catalyst in an organic solvent, water or a mix of water and organic solvent (see for example ChemCatChem, 5(10), 2939-2945; 2013; Organic Letters, 17(12), 2878-2881; 2015). As metal catalyst can be used for example Ru, Ir, and Pd, with or without ligands such as phosphines, phosphates, cyclooctadiene, diamines and imidazoles. The reaction can take place at temperature from 0° C. to 100° C. Preferable organic solvent are methanol, acetone, dichloromethane, 2,2,2-trifluoroethanol or DMF. The reaction can also take place the presence of an acid for example HCO₂H, trifluoro acetic acid and acetic acid.

Compounds of the formula II can be provided e.g. starting from alcohols of type III with nitriles of type IV in the presence of an acid in an organic solvent (see for example US 2008/0275242 or WO2005/070917). Preferably, sulfuric acid or a sulfonic acid, in particular triflic acid, are used as acid. Most suitable solvents are hydrocarbons, preferably benzene or dichloromethane.

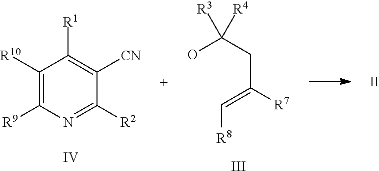

Depending on the nature of the starting materials, the reaction is performed at a temperature from −40° C. to 200° C., in particular from −10° C. to 120° C., more specifically from 0° C. to 100° C., even more specifically from room or ambient temperature (about 23° C.) to 80° C.

Nitriles of type IV are either commercially available or can be prepared by a skilled person from the corresponding halides following literature procedures (see, for example Journal of Organic Chemistry, 76(2), 665-668; 2011; Angewandte Chemie, International Edition, 52(38), 10035-10039; 2013; WO2004/013094).

Alcohols of type III can be prepared as described below. A skilled person will realize that compounds of type V can be reacted with organometallic reagents, preferably alkyl Grignard or alkyl-Lithium reagents, in ethereal solvents, preferably THF at low temperatures and under inert conditions to furnish compounds of type III.

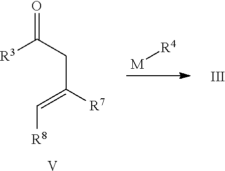

Alternatively, alcohols of type III can be prepared from epoxides Va and compounds VI (see below):

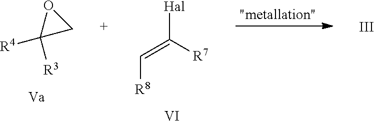

The metalation reaction may preferably be carried out using Lithium-organic compounds, such as for example n-butyl lithium, sec-butyl lithium or tert-butyl lithium to result in an exchange of halogen by lithium. Also suitable is the reaction with magnesium resulting in the formation of the respective Grignard reagents. A further possibility is the use of other Grignard reagents such as isopropyl-magnesium-bromide instead of Mg.

A typical preparation of compounds of type III can be achieved by reacting compounds of type VII with organometallic reagents, preferably alkyl Grignard or alkyl-Lithium reagents, in ethereal solvents, preferably THF at low temperatures and under inert conditions to furnish compounds of type IV as previously reported (see for example WO2012051036; WO2011042918).

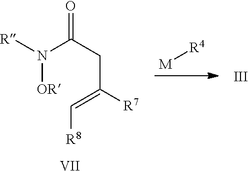

Compounds of type VII can be accessed by reacting a carbonyl compound of type VIII, preferably a carboxylic acid (X═OH) or an acid chloride (X═Cl), with NH(OR′)R″, wherein R′ and R″ are selected from (C₁-C₄)-alkyl, most preferably being methyl, in an organic solvent, preferably THF or dichloromethane. Typically the reaction is performed in a range between 0° C. and ambient temperature in the presence of an organic base, preferably NEt₃ or pyridine (see e.g. US 20130324506; Tetrahedron: Asymmetry, 17(4), 508-511; 2006). If X═OH, the addition of an activating reagent, preferably a carbodiimide, may be preferred (see for example ChemMedChem, 7(12), 2101-2112; 2012; 2011038204; Journal of Organic Chemistry, 76(1), 164-169; 2011).

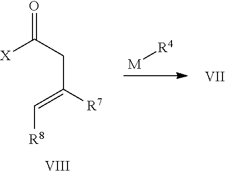

If required, compounds of type VIII can be prepared from the corresponding aryl halides of type IX (Hal is halogen, preferably Br or I). As described (Tetrahedron, 68(9), 2113-2120; 2012; Chemical Communications (Cambridge, United Kingdom), 49(60), 6767-6769; 2013), aryl halides VI will react with compounds of type IX in the presence of a transition metal catalyst, preferably a copper(I) salt, in an organic solvent, preferably DMF or DMSO, at elevated temperatures. Typically a base, preferably potassium phosphate, is added.

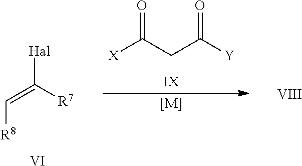

If appropriate, compounds of type III can be prepared as follows. A known or commercially available carbonyl compound can be reacted with an organometallic reagent of type X, preferably a Grignard or an organolithium reagent, readily prepared by a skilled person. Preferably, the reaction is performed in a temperature range from −78° C. to room temperature under inert conditions in an ethereal solvent.

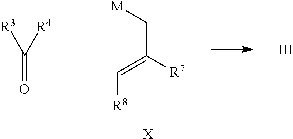

Alternatively compounds II can also be accessed by reacting a nitrile IV with an olefin IIIa under acidic conditions as described elsewhere (U.S. Pat. No. 7,632,783, B2, page 60, method A).

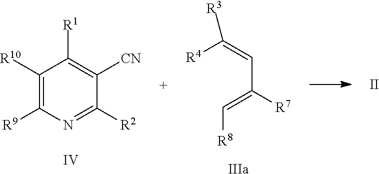

Alternatively compounds II can be prepared via intramolecular reaction of amide XI with an electron-rich heterocycle or aryl group. The intramolecular cyclization will take place in the presence of a dehydrating agent in an organic solvent (WO 2008143263, Synthetic Communications 2007, 37, 1331-1338.). Preferably, phosphoryl chloride (POCl₃), POCl₃/P₂O₅, H₃PO₄/P₂O₅, SnCl₄ or BF₃ are used as dehydrating agent. Most suitable solvents are hydrocarbons, preferably benzene, toluene or acetonitrile. Alternatively halogenated solvents can be used, for example dichloromethane, chloroform or chlorobenzene.

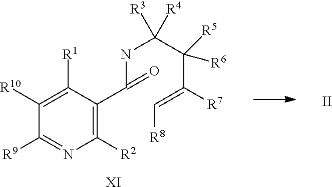

Depending on the nature of starting materials, the reaction is performed at temperature from −40° c. to 200° C., in particular from −10° C. to 120° C., more specifically from 0° C. to 100° C., even more specifically from room temperature to 100° C.

Amides of type XI can accessed by reacting a carbonyl of type XII, preferably a carboxylic acid (X═OH) or an acid chloride (X═Cl), with an amines of type XIII in an organic solvent, preferably THF or dichloromethane. Typically the reaction is performed in a range between 0° C. and room temperature in the presence of an organic base, preferably N(C₂H₅)₃ or pyridine (see e.g. WO 8303968). If X═OH, the addition of an activating agent, preferably a carbodiimide or acid chloride, may be preferred (see e.g Bioorganic & Medicinal Chemistry, 2010, 18, 3088-3115).

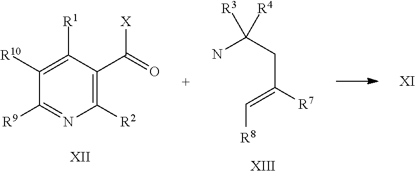

If required, compounds of type XIII can be synthesized from the correspond nitriles. As described Synlett. 2007, 4 652-654 or Tetrahedron 2012, 68, 2696-2703, nitriles will react with organometallic agents X, preferably Grignard or Lithium reagent, in ethereal solvents, preferably THF at low temperature and under inert conditions to furnish compounds of type XIII. The synthesis of compounds of type XIII can take place in two steps or one pot.

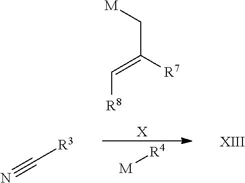

Alternatively, amines of type XIII can synthesized via formation of the correspond carboxylic azide and quench with water (Journal of the American Chemical Society, 1949, 71, 2233-7; Journal of the American Chemical Society, 1990, 112, 297-304) or via Grignard addition to imine (Tetrahedron Letters, 1992, 33, 1689-92; US20030216325)

Compound of type II can be also synthesized via Suzuki coupling of halides of type XIV with a boronic acid XV (see for example, Journal of Fluorine Chemistry, 2010, 131, 856-860); wherein R³¹ and R⁴¹ together with the groups they are attached to form a tetramethyl-1,3,2-dioxaborolane-ring or independently from one another mean hydrogen or C₁-C₆-alkyl to yield compounds I.A.1.

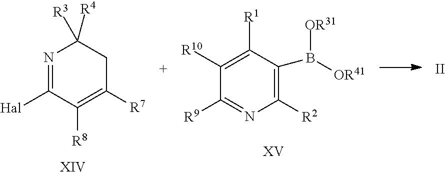

Compounds of type XIV, wherein Hal is halogen, preferably chloro and bromo, can be obtained by transformation of an amide of type XVI with a halogenating reagent, such as phosphorus oxachloride, phosphorus pentachloride, phosphoric trichloride, phosphorus oxybromide, thionyl chloride or Vilsmeier reagent. The reaction takes place in the presence of an organic solvent, preferably THF, benzene, CCl₄, or dichloromethane. Typically the reaction is performed in a range between 0° C. to 180° C. (see as reference, Journal of Medicinal Chemistry, 2004, 47, 663-672; Journal of Organic Chemistry, 1980, 45, 80-89; Bulletin des Societes Chimiques Belges, 1991, 100, 169-174).

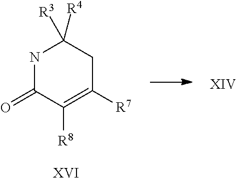

Amides of type XVI can be prepared from compounds of type XVII, wherein R^{x} is a C₁-C₆-alkyl. The reaction takes places in the presence of acid, preferably acetic acid, HCl, triflic acid or a mixture of sodium acetate and acetic acid. Typically the reaction in performed net or in polar solvents, preferably in water, methanol or acetonitrile (see WO2016/156085; Pharmaceutical Chemistry Journal, 2005, 39, 405-408).

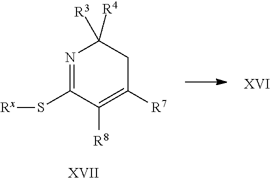

Alternatively, compounds of type XIV can be direct synthesized from compounds of type XVII in the presence of a halogenating reagent, such as sulfonyl chloride. The reaction takes places neat or in organic solvents, such as chloroform, dichloromethane or acetonitrile, in a range of temperature from 0° C. to room temperature (see, Tetrahedrons Letters, 2010, 51, 4609; Tetrahedron Letters, 1986, 27(24), 2743-6).

Compounds of type XVII can also be obtained by the reaction of alcohol III or alkene IIIa and a C₁-C₆-alkyl thiocyanate under acidic conditions, see for example Bioorganic & Medicinal Chemistry Letters, 2013, 23(7), 2181-2186; Pharmaceutical Chemistry Journal, 2005, 39, 405-408. Preferably acids are sulfuric acid, HCl or trific acid. The reaction takes place most preferably in water, dichloromethane, toluene or a mixture of solvents, in a range of temperatures from 0° C. to 110° C.

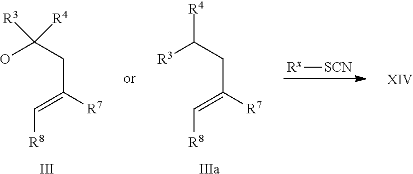

Compounds of type XIVa can be synthesized via ring expansion of oxime XVIII in the presence of an acid. Most suitable acids are for example, sulfuric acid, polyphosphoric acid or POCl₃. Typically the reaction in performed net or in a polar solvents, preferably in water, methanol or acetonitrile (see Bioorganic & Medicinal Chemistry Letters, 2002, 12(3), 387-390; Medicinal Chemistry Research, 2015, 24(2), 523-532).

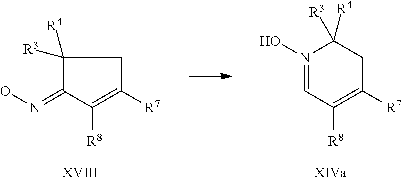

Oxime of type XVIII can be easily prepared from ketone of type XIX in the presence of hydroxylamine or hydroxylamine hydrochloride in polar solvents such as water, pyridine, ethanol or methanol. The reaction can take place in the presence of absence of a base, such as sodium acetate or sodium hydroxide, in a range of temperatures from room temperature to 120° C. (Journal of Organic Chemistry, 2016, 81(1), 336-342).

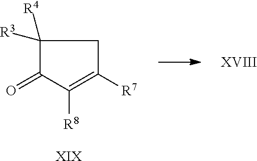

Ketone of type XIX are either commercial available or readily prepared by a skilled person.

Alternatively compounds II-3 can be synthesized from compounds XX, which are commercially available or can be synthesized according to procedures known in literature, in which X¹ denotes for hydrogen or halogen (Cl, Br, I).

Compounds XXI (and X¹ denotes for halogen (Cl, Br, I) or C₁-C₆-alkoxycarbonyl) can be metalated with Grignard-reagents (X³ denotes for Cl, Br or I), for example methyl magnesium-X³, ethyl magnesium-X³, isopropyl-magnesium-X³ and phenyl magnesium X³ among others, or lithium organic reagents like methyl-lithium, ethyl-lithium, butyl-lithium and phenyl-lithium among others, and reacted with compounds XXII to yield derivatives XX, whereas R³¹ and R⁴¹ independently from each other denote for C₁-C₆-alkyl, C₂-C₆-alkenyl, C₂-C₆-alkynyl, a saturated or partially unsaturated three-, four-, five-, six-, seven-, eight-, nine-, or ten-membered carbocycle or heterocycle, five- or six-membered heteroaryl and aryl.

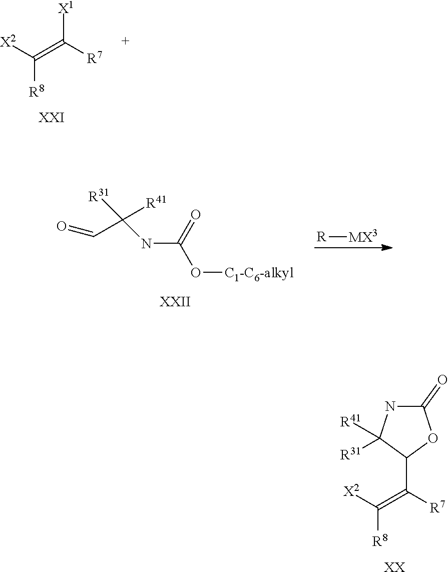

Subsequently compounds XX (X²═Cl, Br, I) can be reacted with carbon monoxide yielding esters XXIII following published literature (Science of Synthesis (2014), 2, 67-93; Comprehensive Inorganic Chemistry 11 (2013), 6, 1-24; RSC Catalysis Series (2015), 21 (New Trends in Cross-Coupling), 479-520; Metal-catalyzed Cross-Coupling Reactions and More (Editor: A. De Meijere) (2014), 1, 133-278; Domino Reactions (Editor L. Tietze) (2014), 7-30; Synthesis 2014, 46 (13), 1689-1708; RSC Advances (2014), 4 (20), 10367-10389), for example using Pd-catalyst (i.e. Pd(dppf)Cl₂ ([1,1′-bis(diphenylphosphino)ferrocene]dichloropalladium(II)) and sodium methanolat in methanol under elevated pressure (10-200 bar) of carbon monoxide.

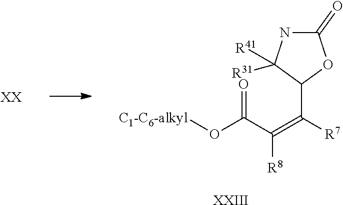

Compounds XXIII can be hydrolyzed using acidic or basic conditions, for example hydrochloric or sulfuric acid, or sodium or potassium carbonate, hydrogen carbonate or hydroxide in water or solvent mixtures with water and alcoholic solvents (preferably methanol, ethanol, isopropanol), or acetonitrile, acetone, dimethylformamide or N-methyl pyrrolidine, at temperatures from 0° C. to 100° C. yielding intermediates XXIV.

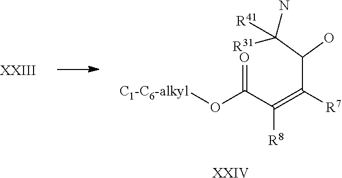

Intermediates XXIV can be activated with reagents like HATU (1-[Bis(dimethylamino)methylene]-1H-1,2,3-triazolo[4,5-b]pyridinium 3-oxid hexafluorophosphate), CDI (1,1′-Carbonyldiimidazole), DCC (N,N′-Methanetetraylbis[cyclohexanamine]) and others known in literature (Eur. JOC 2013, 4325; Tetrahedron 2004, 60, 2447; Tetrahedron 2005, 61, 10827; Chem. Soc. Rev. 2009, 38, 606; Chem. Rev. 2011, 111, 6557) to further react and yield compounds XXV.

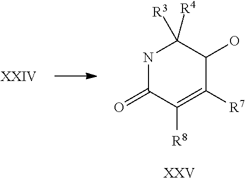

Furthermore compounds XXV are oxidized with MnO₂, hypochlorite, activated DMSO, Cr(VI)-containing reagents or employing other oxidizing conditions known in literature (Korean Chemical Society (2015), 36(12), 2799; Hudlicky, Oxidations in Organic Chemistry, American Chemical Society, Washington D.C., 1990; Acc. Chem. Res. 2002, 35, 774; JACS 1984, 106, 3374; Tetrahedron Letters 56 (2015) 6878; Backvall, Modern Oxidation Methods, Wiley, Weinheim 2004; Tojo, Oxidation of Alcohols to Aldehydes and Ketones, Springer 2006) to provide carbonyl compounds XXVI.

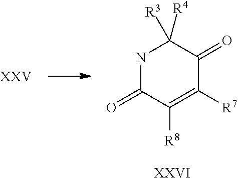

Subsequently the amides XXVI can be transferred into the triflate XXVII by reaction with trifluoromethyl sulfonic anhydride in an inert solvent, like dichloromethane, chloroform, carbon tetrachloride, benzene, toluene or chlorobenzene in the presence of a base, for example an organic base like pyridine, triethylamine or diisopropyl ethylamine or an aqueous base like solutions of sodium or potassium hydroxide, carbonate or hydrogen carbonate in water at temperatures preferably between 0° C. and 100° C.

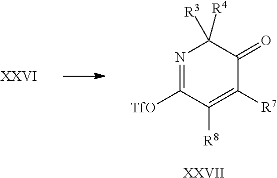

These compounds XXVII are reacted with fluorination reagents (Kirsch, Modern Fluoroorganic Chemistry, Wiley 2013)) like deoxo-fluor (BAST, bis(2-methoxyethyl)aminosulfur trifluoride, Journal of Fluorine Chemistry (2016), 182, 41; Singh, et al. Synthesis 17, 2561, (2002)), DAST (Diethylaminoschwefeltrifluorid, Hudlicky Org. React. 35, 513, (1988)), Fluolead (4-tert-Butyl-2,6-dimethylphenylsulfur trifluoride, WO 2013118915; US 20080039660), Diethylaminodifluorosulfinium tetrafluoroborate (XtalFluor-E) or morpholinodifluorosulfinium tetrafluoroborate (XtalFluor-M) (Journal of organic chemistry (2010), 75(10), 3401) to yield difluoro compounds XXVIII.

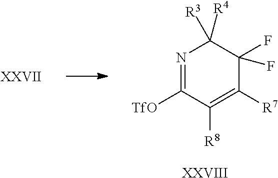

Subsequently these triflates XXVII can be reacted under Suzuki conditions (European Journal of Organic Chemistry (2008), (12), 2013) with boronic acids XV, in which R³¹¹ and R⁴¹¹ together with the groups they are attached to form a tetramethyl-1,3,2-dioxaborolane-ring or independently from one another mean hydrogen or C₁-C₆-alkyl to yield compounds II.

Alternatively, compounds of type II can also be obtained intramolecular cyclization of amines of type XXIX in the presence of an acid. Most preferably acids are HCl, trifluoroacetic acid, acetic acid or sulfuric acid. The reaction is preform in dichloromethane, water, ethanol, THF or chloroform, at temperature from room temperature to 120° C. (see, Synthesis, 1995, (5), 592-604; Heterocycles, 1988, 27(10), 2403-12).

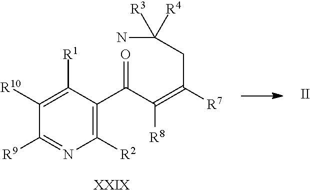

Amines of type XXIX are either commercial available or easily prepared by a skilled person or following the procedures described before.

The N-oxides may be prepared from the inventive compounds according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

In the following, the intermediate compounds are further described. A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

If the synthesis yields mixtures of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Likewise, the term “C₂-C₄-alkyl” refers to a straight-chained or branched alkyl group having 2 to 4 carbon atoms, such as ethyl, propyl (n-propyl), 1-methylethyl (iso-propoyl), butyl, 1-methylpropyl (sec.-butyl), 2-methylpropyl (iso-butyl), 1,1-dimethylethyl (tert.-butyl).

The term “C₁-C₆-halogenalkyl” refers to an alkyl group having 1 or 6 carbon atoms as defined above, wherein some or all of the hydrogen atoms in these groups may be replaced by halogen atoms as mentioned above. Examples are “C₁-C₂-halogenalkyl” groups such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl.

The term “C₁-C₆-hydroxyalkyl” refers to an alkyl group having 1 or 6 carbon atoms as defined above, wherein some or all of the hydrogen atoms in these groups may be replaced by OH groups.

The term “C₁-C₄-alkoxy-C₁-C₄-alkyl” refers to alkyl having 1 to 4 carbon atoms (as defined above), where According to one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 4 carbon atoms (as defined above), where According to one hydrogen atom of the alkyl radical is replaced by a C₁-C₆-alkoxy group (as defined above).

The term “C₂-C₆-alkenyl” refers to a straight-chain or branched unsaturated hydrocarbon radical having 2 to 6 carbon atoms and a double bond in any position. Examples are “C₂-C₄-alkenyl” groups, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond. Examples are “C₂-C₄-alkynyl” groups, such as ethynyl, prop-1-ynyl, prop-2-ynyl (propargyl), but-1-ynyl, but-2-ynyl, but-3-ynyl, 1-methyl-prop-2-ynyl.

The term “C₁-C₆-alkoxy” refers to a straight-chain or branched alkyl group having 1 to 6 carbon atoms which is bonded via an oxygen, at any position in the alkyl group. Examples are “C₁-C₄-alkoxy” groups, such as methoxy, ethoxy, n-propoxy, 1-methylethoxy, butoxy, 1-methyl¬propoxy, 2-methylpropoxy or 1,1-dimethylethoxy.

The term “C₁-C₆-halogenalkoxy” refers to a C₁-C₆-alkoxy radical as defined above, wherein some or all of the hydrogen atoms in these groups may be replaced by halogen atoms as mentioned above. Examples are “C₁-C₄-halogenalkoxy” groups, such as OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chlorothoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoro-propoxy, 2 chloropropoxy, 3-chloropropoxy, 2,3-dichloropropoxy, 2-bromopropoxy, 3 bromopropoxy, 3,3,3-trifluoropropoxy, 3,3,3-trichloropropoxy, OCH₂—C₂F₅, OCF₂—C₂F₅, 1-fluoromethyl-2-fluoroethoxy, 1-chloromethyl-2-chloroethoxy, 1-bromomethyl-2-bromoethoxy, 4-fluorobutoxy, 4-chlorobutoxy, 4-bromobutoxy or nonafluorobutoxy.

The term “C₂-C₆-alkenyloxy” refers to a straight-chain or branched alkenyl group having 2 to 6 carbon atoms which is bonded via an oxygen, at any position in the alkenyl group. Examples are “C₂-C₄-alkenyloxy” groups.

The term “C₂-C₆-alkynyloxy” refers to a straight-chain or branched alkynyl group having 2 to 6 carbon atoms which is bonded via an oxygen, at any position in the alkynyl group. Examples are “C₂-C₄-alkynyloxy” groups.

The term “C₃-C₆-cycloalkyl” refers to monocyclic saturated hydrocarbon radicals having 3 to 6 carbon ring members, such as cyclopropyl, cyclobutyl, cyclopentyl, cyclohexyl. Accordingly, a saturated three-, four-, five-, six-, seven-, eight-, nine or ten-membered carbocyclyl or carbocycle is a “C₃-C₁₀-cycloalkyl”.

The term “C₃-C₆-cycloalkenyl” refers to a monocyclic partially unsaturated 3-, 4- 5- or 6-membered carbocycle having 3 to 6 carbon ring members and at least one double bond, such as cyclopentenyl, cyclopentadienyl, cyclohexadienyl. Accordingly, a partially unsaturated three-, four-, five-, six-, seven-, eight-, nine or ten-membered carbocyclyl or carbocycle is a “C₃-C₁₀-cycloalkenyl”.

The term “C₃-C₈-cycloalkyl-C₁-C₄-alkyl” refers to alkyl having 1 to 4 carbon atoms (as defined above), where According to one hydrogen atom of the alkyl radical is replaced by a cycloalkyl radical having 3 to 8 carbon atoms (as defined above).

The term “C₁-C₆-alkylthio” as used herein refers to straight-chain or branched alkyl groups having 1 to 6 carbon atoms (as defined above) bonded via a sulfur atom. Accordingly, the term “C₁-C₆-halogenalkylthio” as used herein refers to straight-chain or branched halogenalkyl group having 1 to 6 carbon atoms (as defined above) bonded through a sulfur atom, at any position in the halogenalkyl group.

The term “C(═O)—C₁-C₆-alkyl” refers to a radical which is attached through the carbon atom of the group C(═O) as indicated by the number valence of the carbon atom. The number of valence of carbon is 4, that of nitrogen is 3. Likewise the following terms are to be construed: NH(C₁-C₄-alkyl), N(C₁-C₄-alkyl)₂, NH(C₃-C₆-cycloalkyl), N(C₃-C₆-cycloalkyl)₂, C(═O)—NH(C₁-C₆-alkyl), C(═O)—N(C₁-C₆-alkyl)₂.

The term “saturated or partially unsaturated three-, four-, five-, six-, seven-, eight-, nine or ten-membered heterocyclyl or heterocycle, wherein the heterocyclyl or heterocycle contains 1, 2, 3 or 4 heteroatoms selected from N, O and S” is to be understood as meaning both saturated and partially unsaturated heterocycles, wherein the ring member atoms of the heterocycle include besides carbon atoms 1, 2, 3 or 4 heteroatoms independently selected from the group of O, N and S. For example:

a 3- or 4-membered saturated heterocycle which contains 1 or 2 heteroatoms from the group consisting of O, N and S as ring members such as oxirane, aziridine, thiirane, oxetane, azetidine, thiethane, [1,2]dioxetane, [1,2]dithietane, [1,2]diazetidine; and
a 5- or 6-membered saturated or partially unsaturated heterocycle which contains 1, 2 or 3 heteroatoms from the group consisting of O, N and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and
a 7-membered saturated or partially unsaturated heterocycle such as tetra- and hexahydroazepinyl, such as 2,3,4,5-tetrahydro[1H]azepin-1-,-2-,-3-,-4-,-5-,-6- or -7-yl, 3,4,5,6-tetrahydro[2H]azepin-2-,-3-,-4-,-5-,-6- or -7-yl, 2,3,4,7-tetrahydro[1H]azepin-1-,-2-,-3-,-4-,-5-,-6- or -7-yl, 2,3,6,7-tetrahydro[1H]azepin-1-,-2-,-3-,-4-,-5-,-6- or -7-yl, hexahydroazepin-1-,-2-,-3- or -4-yl, tetra- and hexahydrooxepinyl such as 2,3,4,5-tetrahydro[1H]oxepin-2-,-3-,-4-,-5-,-6- or -7-yl, 2,3,4,7-tetrahydro[1H]oxepin-2-,-3-,-4-,-5-,-6- or -7-yl, 2,3,6,7-tetrahydro[1H]oxepin-2-, -3-,-4-,-5-,-6- or -7-yl, hexahydroazepin-1-,-2-,-3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “substituted” refers to substituted with 1, 2, 3 or up to the maximum possible number of substituents.

The term “5- or 6-membered heteroaryl” or “5- or 6-membered heteroaromatic” refers to aromatic ring systems including besides carbon atoms, 1, 2, 3 or 4 heteroatoms independently selected from the group consisting of N, O and S, for example,

a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or
a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

Agriculturally acceptable salts of the inventive compounds encompass especially the salts of those cations or the acid addition salts of those acids whose cations and anions, respectively, have no adverse effect on the fungicidal action of said compounds. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting such inventive compound with an acid of the corresponding anion, preferably of hydrochloric acid, hydrobromic acid, sulfuric acid, phosphoric acid or nitric acid.

The inventive compounds can be present in atropisomers arising from restricted rotation about a single bond of asymmetric groups. They also form part of the subject matter of the present invention.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

In the following, particular embodiments of the inventive compounds are described. Therein, specific meanings of the respective substituents are further detailed, wherein the meanings are in each case on their own but also in any combination with one another, particular embodiments of the present invention.

Furthermore, in respect of the variables, generally, the embodiments of the compounds I also apply to the intermediates.

R¹ according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl;
wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted with one, two, three, four or five substituents R^{x1} independently selected from C₁-C₄-alkyl, halogen, OH, CN, C₁-C₄-halogenalkyl, C₁-C₄-alkoxy and C₁-C₄-halogenalkoxy;
wherein the acyclic moieties of R¹ are unsubstituted or substituted with identical or different groups R^{1a} which independently of one another are selected from:
R^{1a} halogen, OH, CN, C₁-C₆-alkoxy, C₃-C₆-cycloalkyl, C₃-C₆-halogencycloalky, C₁-C₄-halogenalkoxy, C₁-C₆-alkylthio, aryl and phenoxy, wherein the aryl and phenoxy group is unsubstituted or unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R¹ are unsubstituted or substituted with identical or different groups R^{1b} which independently of one another are selected from:
R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

For every R¹ that is present in the inventive compounds, the following embodiments and preferences apply independently of the meaning of any other R¹ that may be present in the ring.

According to one embodiment of formula I, R¹ is H, halogen or C₁-C₆-alkyl, in particular H, CH₃, Et, F, Cl, more specifically H, CH₃, F or Cl most preferred H, F or Cl.

According to another embodiment of formula I, R¹ is hydrogen.

According to still another embodiment of formula I, R¹ is halogen, in particular Br, F or Cl, more specifically F or Cl.

According to another embodiment of formula I, R¹ is F

According to another embodiment of formula I, R¹ is Cl

According to another embodiment of formula I, R¹ is Br.

According to still another embodiment of formula I, R¹ is OH.

According to still another embodiment of formula I, R¹ is CN.

According to still another embodiment of formula I, R¹ is NO₂.

According to still another embodiment of formula I, R¹ is SH.

According to still another embodiment of formula I R¹ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x1} independently selected from C₁-C₄-alkyl, halogen, OH, CN, C₁-C₄-halogenalkyl, C₁-C₄-alkoxy, or C₁-C₄-halogenalkoxy. In particular C₁-C₄-alkyl, such as NHCH₃ and N(CH₃)₂. In particular R^{x} is C₁-C₄-alkyl, and phenyl that is substituted with one CH₃, more specifically SO₂—R^{x} is CH₃ and tosyl group (“Ts”).

According to still another embodiment of formula I, R¹ is C₁-C₆-alkyl, in particular C₁-C₄-alkyl, such as CH₃ or CH₂CH₃.

According to still another embodiment of formula I, R¹ is C₁-C₆-halogenalkyl, in particular C₁-C₄-halogenalkyl, such as CF₃, CHF₂, CH₂F, CCl₃, CHCl₂, CH₂Cl, CF₃CH₂, CCl₃CH₂ or CF₂CHF₂.

According to still another embodiment of formula I, R¹ is C₂-C₆-alkenyl or C₂-C₆-halogenalkenyl, in particular C₂-C₄-alkenyl or C₂-C₄-halogenalkenyl, such as CH═CH₂, C(CH₃)═CH₂, CH═CCl₂, CH═CF₂, CCl═CCl₂, CF═CF₂, CH═CH₂, CH₂CH═CCl₂, CH₂CH═CF₂, CH₂CCl═CCl₂, CH₂CF═CF₂, CCl₂CH═CCl₂, CF₂CH═CF₂, CCl₂CCl═CCl₂, or CF₂CF═CF₂.

According to still another embodiment of formula I, R¹ is C₂-C₆-alkynyl or C₂-C₆-halogenalkynyl, in particular C₂-C₄-alkynyl or C₂-C₄-halogenalkynyl, such as C≡CH, C≡CCl, C≡CF. CH₂C≡CH, CH₂C≡CCl, or CH₂C≡CF.

According to still another embodiment of formula I, R¹ is C₁-C₆-alkoxy, in particular C₁-C₄-alkoxy, more specifically C₁-C₂-alkoxy such as OCH₃ or OCH₂CH₃.

According to still another embodiment of formula I, R¹ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R¹ is C₃-C₆-cycloalkyl, in particular cyclopropyl.

According to still another embodiment of formula I, R¹ is C₃-C₆-cycloalkyl, for example cyclopropyl, substituted with one, two, three or up to the maximum possible number of identical or different groups R^{1b} as defined and preferably herein.

According to still another embodiment of formula I, R¹ is C₃-C₆-halogencycloalkyl. In a special embodiment R¹ is fully or partially halogenated cyclopropyl.

According to still another embodiment of formula I, R¹ is unsubstituted aryl or aryl that is substituted with one, two, three or four R^{1b}, as defined herein. In particular, R¹ is unsubstituted phenyl or phenyl that is substituted with one, two, three or four R^{1b}, as defined herein.

According to still another embodiment of formula I, R¹ is unsubstituted 5- or 6-membered heteroaryl. According to still a further embodiment, R¹ is 5- or 6-membered heteroaryl that is substituted with one, two or three R^{1b}, as defined herein.

According to still another embodiment of formula I, R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R¹ are not further substituted or carry one, two, three, four or five identical or different groups R^{1a} as defined below and wherein the carbocyclic, heteroaryl and aryl moieties of R¹ are not further substituted or carry one, two, three, four or five identical or different groups R^{1b} as defined below.

According to still another embodiment of formula I, R¹ is independently selected from hydrogen, halogen, CN, OH, C₁-C₆-alkyl, C₁-C₆-alkoxy, C₆-alkenyl, C₂-C₆-alkynyl, C₃-C₆-cycloalkyl, wherein the acyclic and cyclic moieties of R¹ are unsubstituted or substituted by halogen.

According to still another embodiment of formula I, R¹ is independently selected from hydrogen, halogen, CN, OH, C₁-C₆-alkyl, C₁-C₆-halogenalkyl, C₁-C₆-alkoxy and C₁-C₆-halogenalkoxy, in particular independently selected from H, F, Cl, Br, CN, OH, C₁-C₄-alkyl, C₁-C₄-halogenalkyl, C₁-C₄-alkoxy and C₁-C₄-halogenalkoxy.

According to still another embodiment of formula I, R¹ is independently selected from H, CN, halogen or C₁-C₆-alkyl, in particular H, CN, CH₃, Et, F, Cl, more specifically H, CN, CH₃, F or Cl most preferred H, CH₃, F or Cl.

R^{1a} are the possible substituents for the acyclic moieties of R¹.

R^{1a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

In to one embodiment R^{1a} is independently selected from halogen, OH, CN, C₁-C₂-alkoxy, C₃-C₆-cycloalkyl, C₃-C₆-halogencycloalkyl and C₁-C₂-halogenalkoxy. Specifically, R^{1a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{1a} is independently selected from halogen, such as F, Cl, Br and I, more specifically F, Cl and Br.

According to still another embodiment of formula I, R^{1a} is independently selected from OH, C₃-C₆-cycloalkyl, C₃-C₆-halogencycloalkyl and C₁-C₂-halogenalkoxy. Specifically, R^{1a} is independently selected from OH, cyclopropyl and C₁-C₂-halogenalkoxy.

According to still another embodiment of formula I, R^{1a} is independently selected from aryl and phenoxy, wherein the aryl group is unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

R^{1b} are the possible substituents for the carbocyclic, heteroaryl and aryl moieties of R¹.

R^{1b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;

According to one embodiment thereof R^{1b} is independently selected from halogen, CN, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{1b} is independently selected from F, Cl, Br, OH, CN, CH₃, OCH₃, CHF₂, OCHF₂, cyclopropyl, 1-F-cyclopropyl, 1-Cl-cyclopropyl, 1,1-F₂-cyclopropyl, 1,1-Cl₂-cyclopropyl, OCF₃, and OCHF₂.

According to still another embodiment thereof R^{1b} is independently selected from halogen, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{1b} is independently selected from halogen, CN, OH, CH₃, CHF₂, OCHF₂, OCF₃, OCH₃, cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from F, Cl, OH, CH₃, OCH₃, CHF₂, OCH₃, cyclopropyl, 1-F-cyclopropyl, 1-Cl-cyclopropyl, 1,1-F₂-cyclopropyl, 1,1-Cl₂-cyclopropyl, OCHF₂ and OCF₃.

R^{x} in the substituent NH—SO₂—R^{x} is in each case independently selected from C₁-C₄-alkyl, C₁-C₄-halogenalkyl, unsubstituted aryl and aryl that is substituted with one, two, three, four or five substituents R^{x1} independently selected from C₁-C₄-alkyl, halogen, OH, CN, C₁-C₄-halogenalkyl, C₁-C₄-alkoxy and C₁-C₄-halogenalkoxy. In particular, R^{x} is in each case independently selected from C₁-C₄-alkyl, halogen, OH, CN and phenyl that is substituted with one, two or three R^{x1} independently selected from C₁-C₂-alkyl, more specifically R^{x} is in each case independently selected from C₁-C₄-alkyl and phenyl that is substituted with one CH₃, more specifically SO₂—R^{x} is the tosyl group (“Ts”).

Particularly preferred embodiments of R¹ according to the invention are in Table P1 below, wherein each line of lines P1-1 to P1-16 corresponds to one particular embodiment of the invention. Thereby, for every R¹ that is present in the inventive compounds, these specific embodiments and preferences apply independently of the meaning of any other R¹ that may be present in the ring:

TABLE P1
“Ts” in the table stands for the tosylgroup SO₂-(p-CH₃)phenyl.

| No. | R¹ |
|---|---|
| P1-1 | H |
| P1-2 | Cl |
| P1-3 | F |
| P1-4 | Br |
| P1-5 | OH |
| P1-6 | CN |
| P1-7 | NO₂ |
| P1-8 | CH₃ |
| P1-9 | CH₂CH₃ |
| P1-10 | CH₃ |
| P1-11 | CHF₂ |
| P1-12 | OCH₃ |
| P1-13 | OCH₂CH₃ |
| P1-14 | OCF₃ |
| P1-15 | OCHF₂ |
| P1-16 | NH-Ts |

R² according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy and C₁-C₄-halogenalkoxy;
   wherein the acyclic moieties of R² are unsubstituted or substituted with identical or different groups R^{2a} which independently of one another are selected from:
   R^{2a} halogen, OH, CN, C₁-C₆-alkoxy, C₃-C₆-cycloalkyl, C₃-C₆-halogencycloalky, C₁-C₄-halogenalkoxy, C₁-C₆-alkylthio, aryl and phenoxy, wherein the aryl group is unsubstituted or substituted with R^{21a} selected from the group consisting of halogen, OH, C₁-C₄-alkyl, C₁-C₄-halogenalkyl, C₁-C₄-alkoxy and C₁-C₄-halogenalkoxy;
   wherein the carbocyclic, heteroaryl and aryl moieties of R² are unsubstituted or substituted with identical or different groups R^{2b} which independently of one another are selected from:
   R^{2b} halogen, OH, CN, C₁-C₄-alkyl, C₁-C₄-alkoxy, C₁-C₄-halogenalkyl, C₃-C₆-cycloalkyl, C₃-C₆-halogencycloalky, C₁-C₄-halogenalkoxy and C₁-C₆-alkylthio.

For every R² that is present in the inventive compounds, the following embodiments and preferences apply independently of the meaning of the other R² that may be present in the ring.

According to one embodiment of formula I, R² is H, halogen or C₁-C₆-alkyl, in particular H, CH₃, Et, F, Cl, more specifically H, CH₃, F or Cl most preferred H, F or Cl.

According to another of formula I, R² is halogen, in particular Br, F or Cl, more specifically F or Cl.

According to another embodiment of formula I, R² is F

According to another embodiment of formula I, R² is Cl

According to another embodiment of formula I, R² is Br.

According to still another embodiment of formula I, R² is hydrogen.

According to still another embodiment of formula I, R² is OH.

According to still another embodiment of formula I, R² is CN.

According to still another embodiment of formula I, R² is NO₂.

According to still another embodiment of formula I, R² is SH.

In a further specific embodiment R² is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy, or C₁-C₄-halogenalkoxy. In particular C₁-C₄-alkyl, such as NHCH₃ and N(CH₃)₂. In particular R^{x} is C₁-C₄-alkyl, and phenyl that is substituted with one CH₃, more specifically SO₂—R^{x} is CH₃ and tosyl group (“Ts”).

According to still another embodiment of formula I, R² is C₁-C₆-alkyl, in particular C₁-C₄-alkyl, such as CH₃ or CH₂CH₃.

According to still another embodiment of formula I, R² is C₁-C₆-halogenalkyl, in particular C₁-C₄-halogenalkyl, such as CF₃, CHF₂, CH₂F, CCl₃, CHCl₂, CH₂Cl, CF₃CH₂, CCl₃CH₂ or CF₂CHF₂.

According to still a further embodiment, R² is C₂-C₆-alkenyl or C₂-C₆-halogenalkenyl, in particular C₂-C₄-alkenyl or C₂-C₄-halogenalkenyl, such as CH═CH₂, CH═CCl₂, CH═CF₂, CCl═CCl₂, CF═CF₂, CH═CH₂, CH₂CH═CCl₂, CH₂CH═CF₂, CH₂CCl═CCl₂, CH₂CF═CF₂, CCl₂CH═CCl₂, CF₂CH═CF₂, CCl₂CCl═CCl₂, or CF₂CF═CF₂.

According to still a further embodiment, R² is C₂-C₆-alkynyl or C₂-C₆-halogenalkynyl, in particular C₂-C₄-alkynyl or C₂-C₄-halogenalkynyl, such as C≡CH, C≡CCl, C≡CF. CH₂C≡CH, CH₂C≡CCl, or CH₂C≡CF.

According to still another embodiment of formula I, R² is C₁-C₆-alkoxy, in particular C₁-C₄-alkoxy, more specifically C₁-C₂-alkoxy such as OCH₃ or OCH₂CH₃.

According to still another embodiment of formula I, R² is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

In a further specific embodiment R² is C₃-C₆-cycloalkyl, in particular cyclopropyl.

In a further specific embodiment, R² is C₃-C₆-cycloalkyl, for example cyclopropyl, substituted with one, two, three or up to the maximum possible number of identical or different groups R^{2b} as defined and preferably herein.

According to still another embodiment of formula I, R² is C₃-C₆-halogencycloalkyl. In a special embodiment R² is fully or partially halogenated cyclopropyl.

According to still another embodiment of formula I, R² is unsubstituted aryl or aryl that is substituted with one, two, three or four R^{2b}, as defined herein. In particular, R² is unsubstituted phenyl or phenyl that is substituted with one, two, three or four R^{2b}, as defined herein.

According to still another embodiment of formula I, R² is unsubstituted 5- or 6-membered heteroaryl. According to still a further embodiment, R² is 5- or 6-membered heteroaryl that is substituted with one, two or three R^{2b}, as defined herein.

According to still another embodiment of formula I, R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R² are not further substituted or carry one, two, three, four or five identical or different groups R^{2a} as defined below and wherein the cycloalkyl moieties of R² are not further substituted or carry one, two, three, four or five identical or different groups R^{2b} as defined below.

According to still another embodiment of formula I, R² is independently selected from hydrogen, halogen, CN, OH, C₁-C₆-alkyl, C₁-C₆-alkoxy, C₆-alkenyl, C₂-C₆-alkynyl, C₃-C₆-cycloalkyl, wherein the acyclic and cyclic moieties of R² are unsubstituted or substituted by halogen.

According to still another embodiment of formula I, R² is independently selected from hydrogen, halogen, OH, C₁-C₆-alkyl, C₁-C₆-halogenalkyl, C₁-C₆-alkoxy and C₁-C₆-halogenalkoxy, in particular independently selected from H, F, Cl, Br, CN, OH, C₁-C₄-alkyl, C₁-C₄-halogenalkyl, C₁-C₄-alkoxy and C₁-C₄-halogenalkoxy.

According to still another embodiment of formula I, R² is independently selected from H, CN, halogen or C₁-C₆-alkyl, in particular H, CN, CH₃, Et, F, Cl, more specifically H, CN, CH₃, F or Cl most preferred H, CH₃, F or Cl.

R^{2a} are the possible substituents for the acyclic moieties of R².

R^{2a} according to the invention is independently selected from halogen, OH, CN, C₁-C₆-alkoxy, C₃-C₆-cycloalkyl, C₃-C₆-halogencycloalky, C₁-C₄-halogenalkoxy, C₁-C₆-alkylthio, aryl and phenoxy, wherein the aryl and phenoxy group is unsubstituted or substituted with R^{22a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

According to one embodiment R^{2a} is independently selected from halogen, OH, CN, C₁-C₂-alkoxy, C₃-C₆-cycloalkyl, C₃-C₆-halogencycloalky and C₁-C₂-halogenalkoxy. Specifically, R^{2a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{2a} is independently selected from halogen, such as F, Cl, Br and I, more specifically F, Cl and Br.

According to still another embodiment of formula I, R^{2a} is independently selected from OH, C₃-C₆-cycloalkyl, C₃-C₆-halogencycloalky and C₁-C₂-halogenalkoxy. Specifically, R^{2a} is independently selected from OH, cyclopropyl and C₁-C₂-halogenalkoxy.

According to still another embodiment of formula I, R^{2a} is independently selected from aryl and phenoxy, wherein the aryl and phenoxy group is unsubstituted or substituted with R^{22a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

R^{2b} are the possible substituents for the carbocyclic, heteroaryl and aryl moieties of R².

R^{2b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalky, C₁-C₄-halogenalkoxy and C₁-C₆-alkylthio;

According to one embodiment thereof R^{2b} is independently selected from halogen, CN, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{2b} is independently selected from F, Cl, Br, OH, CN, CH₃, OCH₃, CHF₂, OCHF₂, cyclopropyl, 1-F-cyclopropyl, 1-Cl-cyclopropyl 1,1-F₂-cyclopropyl, 1,1-Cl₂-cyclopropyl, OCF₃, and OCHF₂.

According to still another embodiment thereof R^{2b} is independently selected from halogen, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{2b} is independently selected from halogen, OH, CH₃, OCH₃, CN, CHF₂, OCHF₂, OCF₃, OCH₃ cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from F, Cl, OH, CH₃, OCH₃, CHF₂, OCH₃, cyclopropyl, 1-F-cyclopropyl, 1-Cl-cyclopropyl, 1,1-F₂-cyclopropyl, 1,1-Cl₂-cyclopropyl, OCHF₂ and OCF₃.

Particularly preferred embodiments of R² according to the invention are in Table P2 below, wherein each line of lines P2-1 to P2-16 corresponds to one particular embodiment of the invention. Thereby, for every R² that is present in the inventive compounds, these specific embodiments and preferences apply independently of the meaning of any other R² that may be present in the ring:

TABLE P2
“Ts” in the table stands for the tosylgroup SO₂-(p-CH₃)phenyl.

| No. | R² |
|---|---|
| P2-1 | H |
| P2-2 | Cl |
| P2-3 | F |
| P2-4 | Br |
| P2-5 | OH |
| P2-6 | CN |
| P2-7 | NO₂ |
| P2-8 | CH₃ |
| P2-9 | CH₂CH₃ |
| P2-10 | CF₃ |
| P2-11 | CHF₂ |
| P2-12 | OCH₃ |
| P2-13 | OCH₂CH₃ |
| P2-14 | OCF₃ |
| P2-15 | OCHF₂ |
| P2-16 | NH-Ts |

R³ is in each case independently selected from CH₃, CH₂F, CHF₂ and CF₃.

According to one embodiment R³ is CH₃.

According to another embodiment R³ is CH₂F.

According to still another embodiment R³ is CHF₂.

According to another embodiment R³ is CF₃.

R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted with R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl;

wherein R^{x} is as defined above;
wherein the acyclic moieties of R⁴ are independently not further substituted or carry one, two, three or up to the maximum possible number of identical or different groups R^{4a}, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₃-C₆-cycloalkyl, C₃-C₆-halogencycloalkyl, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbo- or heterocycle, a five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbo-, heterocyclic, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′ and R″ are as defined above; n is 0, 1, 2; and
wherein the carbo-, heterocyclic, heteroaryl and aryl moieties of R⁴ are independently unsubstituted or substituted with identical or different groups R^{4b}, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or carry one, two, three, four or five substituents selected from the group consisting of halogen, OH, C₁-C₄-alkyl, C₁-C₄-halogenalkyl, C₁-C₄-alkoxy and C₁-C₄-halogenalkoxy; and wherein R^{x} and n are as defined above.

According to one embodiment of formula I, R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or

wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy.

According to one embodiment of formula I, R⁴ is selected from substituted C₁-C₆-alkyl, C₁-C₆-halogenalkyl, C₂-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₃-C₆-cycloalkynyl, C₁-C₆-alkoxy, CN, CH(═O), C(═O)C₂-C₆-alkyl, C(═O)O(C₂-C₆-alkyl), CR′═NOR″, C₃-C₆-cycloalkyl, C₃-C₆-halogencycloalkyl, C₁-C₆-alkyl-five- and six-membered heteroaryl, a five- or six-membered heteroaryl, benzyl, aryl; wherein R′ and R″ are defined below; and wherein the acyclic moieties of R⁴ are unsubstituted or substituted with identical or different groups R^{4a} as defined below and wherein wherein the carbocycle, heterocycle and heteroaryl and aryl moieties are unsubstituted or substituted with substituents R^{4b} as defined below.

According to one embodiment of formula I, R⁴ is selected from C₁-C₆-alkyl substituted with halogen, CN, C₁-C₆-alkoxy, C₁-C₄-halogenalkoxy, C₁-C₆-alkylthio, S(O)ₙ—C₁-C₆-alkyl, NH—SO₂—R^{x}, N(C₁-C₆-alkyl)₂, NH—SO₂—R^{x}, NH(C₁-C₆-alkyl), N(C₁-C₆-alkyl)₂, CH(═O), C(═O)C₁-C₆-alkyl, C(═O)O(C₁-C₆-alkyl), a saturated three-, four-, five-, six-, membered carbo- or heterocycle, aryl, a five- or six-membered heteroaryl; wherein R^{x} is defined below; and wherein the acyclic moieties of R⁴ are unsubstituted or substituted with identical or different groups R^{4a} as defined below and wherein wherein the carbocycle, heterocycle and heteroaryl and aryl moieties are unsubstituted or substituted with substituents R^{4b} as defined below.

According to another embodiment of formula I, R⁴ is F

According to another embodiment of formula I, R⁴ is Cl

According to another embodiment of formula I, R⁴ is Br.

According to still another embodiment of formula I, R⁴ is OH.

According to still another embodiment of formula I, R⁴ is CN.

According to still another embodiment of formula I, R⁴ is NO₂.

According to still another embodiment of formula I, R⁴ is SH.

According to still another embodiment of formula I, R⁴ is C₁-C₆-alkylthio, such as SCH₃, SC₂H₅, Sn-propyl, Si-propyl, Sn-butyl, Si-butyl, Stert-butyl, Sn-pentyl, Si-pentyl, CH₂SCH₃ or CH₂SCH₂CH₃.

According to still another embodiment of formula I, R⁴ is C₁-C₆-halogenalkylthio, such as SCF₃, SCCl₃, CH₂SCF₃ or CH₂SCF₃.

According to still another embodiment of formula I, R⁴ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or is substituted with substituents R^{4b} as defined below. According to one embodiment thereof, the carbocycle is unsubstituted. In a particular embodiment, R⁴ is selected from C₁-C₆-halogenalkyl, phenyl-CH₂, halogenphenyl-CH₂, phenyl, halogenphenyl and three-, four-, five- or six-membered carbo- and heterocycle, wherein the carbo- and heterocycle is unsubstituted or is substituted with substituents R^{4b} as defined below.

According to still another embodiment of formula I, R⁴ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or substituted by substituents R^{4b} as defined below. According to one embodiment thereof, the carbo- and heterocycle is unsubstituted. In a particular embodiment, R⁴ is selected from substituted C₁-C₆-halogenalkyl, phenyl, halogenphenyl and three-, four-, five- or six-membered carbo- and heterocycle, wherein the carbo- and heterocycle is unsubstituted or substituted by substituents R^{4b} as defined below.

According to another embodiment of formula I, R⁴ is selected from CN, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, five- or six-membered heteroaryl or aryl which is unsubstituted or substituted with halogen or C₁-C₆-halogenalkyl, and wherein the acyclic moieties of R⁴ are unsubstituted or substituted with identical or different groups R^{4a} as defined below and wherein wherein the carbocycle, heterocycle and heteroaryl and aryl moieties are unsubstituted or substituted with substituents R^{4b} as defined below.

According to still another embodiment of formula I, R⁴ is selected from CN, substituted C₁-C₆-alkyl, C₁-C₆-halogenalkyl, CN, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, phenyl, pyridine, pyrimidine, thiophene, imidazole, triazol, oxadiazol wherein the acyclic moieties of R⁴ are unsubstituted or substituted with identical or different groups R^{4a} as defined below and wherein wherein the carbocycle, heterocycle and heteroaryl and aryl moieties are unsubstituted or substituted with substituents R^{4b} as defined below.

According to still another embodiment of formula I, R⁴ is C₁-C₆-alkyl such as CH₃, C₂H₅, n-propyl, i-propyl, n-butyl, i-butyl, tert-butyl, n-pentyl or i-pentyl.

According to still another embodiment of formula I, R⁴ is C₁-C₆-alkyl such as CH₃.

According to still another embodiment of formula I, R⁴ is C₁-C₆-alkyl such as C₂H₅.

According to still another embodiment of formula I, R⁴ is C₁-C₆-alkyl such as CH₃, C₂H₅, n-propyl, i-propyl, n-butyl, i-butyl, tert-butyl, n-pentyl or i-pentyl which is substituted with at least one group R^{4a}, which independently of one another are selected from:

R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁴ is CH₃ is substituted with at least one group R^{4a}, which independently of one another are selected from:

R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, five-, six- or ten-membered heteroaryl, an aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁴ is C₂H₅ is substituted with at least one group R^{4a}, which independently of one another are selected from:

R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ saturated or partially unsaturated three-, four-, five-, six-, seven-, eight-, nine-, or ten-membered carbocycle or heterocycle, five-, six- or ten-membered heteroaryl, phenyl or phenoxy; wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁴ is CH₂CN.

According to still another embodiment of formula I, R⁴ is CH₂OH.

According to still another embodiment of formula I, R⁴ is C₁-C₆-halogenalkyl, in particular C₁-C₄-halogenalkyl, more specifically C₁-C₂-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I, R⁴ is CH₂F.

According to still another embodiment of formula I, R⁴ is CHF₂.

According to still another embodiment of formula I, R⁴ is CF₃.

According to still a further embodiment of formula I, R⁴ is C₂-C₆-alkenyl, in particular C₂-C₄-alkenyl, such as CH═CH₂, CH₂CH═CH₂ or C(CH₃)C═CH₂.

According to a further specific embodiment of formula I, R⁴ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still a further embodiment of formula I, R⁴ is C₂-C₆-cycloalkenyl, in particular C₂-C₄-cycloalkenyl, such as CH═CH₂-cPr.

According to still a further embodiment of formula I, R⁴ is C₂-C₆-alkynyl or C₂-C₆-halogenalkynyl, in particular C₂-C₄-alkynyl or C₂-C₄-halogenalkynyl, such as C≡CH, C≡C—Cl, C≡C—CH₃, CH₂—C≡CH, CH₂—C≡CCl or CH₂—C≡C—CH₃.

According to still a further embodiment of formula I, R⁴ is C₂-C₆-cycloalkynyl in particular C₂-C₄-cycloalkynyl, such as C≡C-cPr.

According to a further specific embodiment of formula I, R⁴ is C₁-C₆-alkoxy, in particular C₁-C₄-alkoxy, more specifically C₁-C₂-alkoxy such as OCH₃, CH₂CH₃ or CH₂OCH₃.

According to a further specific embodiment of formula I, R⁴ is C₁-C₆-alkyl-C₁-C₆-alkoxy, in particular C₁-C₄-alkyl-C₁-C₄-alkoxy, more specifically C₁-C₂-alkyl-C₁-C₂-alkoxy, such as CH₂OCH₃ or CH₂OCH₂CH₃.

According to a further specific embodiment of formula I, R⁴ is C₂-C₆-alkenyloxy, in particular C₂-C₄-alkenyloxy, more specifically C₁-C₂-alkenyloxy such as OCH═CH₂, OCH₂CH═CH₂ OC(CH₃)CH═CH₂, CH₂OCH═CH₂, or CH₂OCH₂CH═CH₂.

According to a further specific embodiment of formula I, R⁴ is C₂-C₆-alkynyloxy, in particular C₂-C₄-alkynyloxy, more specifically C₁-C₂-alkynyloxy such as OC≡CH, OCH₂C≡CH or CH₂OC≡CH

According to a further specific embodiment of formula I, R⁴ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R⁴ is C₁-C₆-alkyl-C₁-C₆-halogenalkoxy, in particular C₁-C₄-alkyl-C₁-C₄-halogenalkoxy, more specifically C₁-C₂-alkyl-C₁-C₂-halogenalkoxy such as CH₂OCF₃, CH₂OCHF₂, CH₂OCH₂F, CH₂OCCl₃, CH₂OCHCl₂ or CH₂OCH₂Cl, in particular CH₂OCF₃, CH₂OCHF₂, CH₂OCCl₃ or CH₂OCHCl₂.

According to a further specific embodiment of formula I, R⁴ is CH(═O), C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁴ is C₁-C₄-alkyl-CH(═O), C₁-C₄-alkyl-C(═O)C₁-C₆-alkyl, C₁-C₄-alkyl-C(═O)O(C₁-C₆-alkyl), C₁-C₄-alkyl-C(═O)NH(C₁-C₆-alkyl) or C₁-C₄-alkyl-C(═O)N(C₁-C₆-alkyl)₂, especially CH₂CH(═O), CH₂C(═O)C₁-C₆-alkyl, CH₂C(═O)O(C₁-C₆-alkyl), CH₂C(═O)NH(C₁-C₆-alkyl) or CH₂C(═O)N(C₁-C₆-alkyl)₂ wherein alkyl is CH₃, C₂H₅, n-propyl, i-propyl, n-butyl, i-butyl, tert-butyl, n-pentyl or i-pentyl.

According to a further specific embodiment of formula I, R⁴ is CR′═NOR″ such as C(CH₃)═NOCH₃, C(CH₃)═NOCH₂CH₃ or C(CH₃)═NOCF₃.

According to a further specific embodiment of formula I, R⁴ is C₁-C₆-alkyl-NH(C₁-C₄-alkyl) or C₁-C₆-alkyl-N(C₁-C₄-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁴ is C₁-C₆-alkylthio, in particular C₁-C₄-alkoxy, more specifically C₁-C₃-alkylthio such as CH₂SCH₃ or CH₂SCH₂CH₃.

According to a further specific embodiment of formula I, R⁴ is C₁-C₆-alkyl-S(O)ₙ—C₁-C₆-alkyl, wherein alkyl is CH₃, C₂H₅, n-propyl, i-propyl, n-butyl, i-butyl, tert-butyl, n-pentyl or i-pentyl and n is 1,2 or 3.

According to a further specific embodiment of formula I, R⁴ is C₁-C₆-alkyl-S(O)ₙ—C₁-C₆-halogenalkyl, wherein halogenalkyl is CF₃ or CHF₂ and n is 1, 2 or 3.

According to a further specific embodiment of formula I, R⁴ is C₁-C₆-alkyl-S(O)ₙ-aryl, wherein the aryl or phenyl moiety in each case is unsubstituted or substituted with identical or different groups R^{4b} which independently of one another are selected from halogen, C₁-C₂-alkyl, C₁-C₂-alkoxy, C₁-C₂-halogenalkyl, C₁-C₂-halogenalkoxy and S(O)ₙ—C₁-C₆-alkyl, in particular F, Cl, Br, CH₃, OCH₃, CF₃, CHF₂, OCHF₂, OCF₃. According to one embodiment, R⁴ is unsubstituted phenyl. According to another embodiment, R⁴ is phenyl, that is substituted with one, two or three, in particular one, halogen, in particular selected from F, Cl and Br, more specifically selected from F and Cl.

According to a further specific embodiment of formula I, R⁴ is C₁-C₆-alkyl-NH—SO₂—R^{x} wherein R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy, or C₁-C₄-halogenalkoxy, such as CH₂NHSO₂CF₃ or CH₂NHSO₂CH₃.

According to still another embodiment of formula I, R⁴ is selected from C₁-C₆-alkyl which is substituted, a saturated three-, four-, five-, six-, seven-, eight-, nine-, or ten-membered carbocycle, in particular three-, four-, five- or six-membered, wherein the carbocycle is unsubstituted or substituted with substituents R^{4b} as defined below. According to one embodiment thereof, the carbocycle is unsubstituted.

According to one embodiment, R⁴ is selected from C₁-C₆-alkyl, especially CH₂ which is substituted with a 3-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is selected from C₁-C₆-alkyl, especially CH₂ which is substituted with a 4-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is selected from C₁-C₆-alkyl, especially CH₂ which is substituted with a 5-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is selected from C₁-C₆-alkyl, especially CH₂ which is substituted with a 6-membered saturated carbocycle. According to one embodiment thereof, the carbocycle is unsubstituted, i.e. it does not carry any substituent R^{4b}. According to still another embodiment of formula I, it is substituted with R^{4b}.

According to a further specific embodiment of formula I, R⁴ is C₁-C₆-alkylheterocycle, especially CH₂ substituted with a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{4b}. According to still another embodiment of formula I, it is substituted with R^{4b}.

According to a further specific embodiment of formula I, R⁴ is C₁-C₆-alkylheterocycle, especially CH₂ substituted with a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{4b}. According to still another embodiment of formula I, it is substituted with R^{4b}.

According to a further specific embodiment of formula I, R⁴ is C₁-C₆-alkylheterocycle, especially CH₂ substituted by a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{4b}. According to still another embodiment of formula I, it is substituted with R^{4b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{4b}. According to still another embodiment of formula I, it is substituted with R^{4b}.

According to a further specific embodiment of formula I, R⁴ is C₁-C₆-alkylheterocycle, especially CH₂ substituted with a 5-membered saturated heterocycle which contains one N as ring member and optionally one or two groups CH₂ are replaced by C(═O).

According to still another embodiment of formula I, R⁴ is a partially unsaturated three-, four-, five-, six-, seven-, eight-, nine-, or ten-membered carbocycle, in particular three-, four-, five- or six-membered, wherein the carbocycle is unsubstituted or substituted with substituents R^{4b} as defined below. According to one embodiment thereof, the carbocycle is unsubstituted.

According to still another embodiment of formula I, R⁴ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

According to still a further embodiment, R⁴ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

According to still another embodiment of formula I, R⁴ is a saturated three-, four-, five-, six-, seven-, eight-, nine-, or ten-membered carbocycle, in particular three-, four-, five- or six-membered, wherein the carbocycle is unsubstituted or substituted with substituents R^{4b} as defined below. According to one embodiment thereof, the carbocycle is unsubstituted.

According to one embodiment, R⁴ is a 3-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is a 3-membered saturated carbocycle, which is unsubstituted such as cyclopropyl.

According to one embodiment, R⁴ is a 3-membered saturated carbocycle, which is substituted with halogen, more specifically by F, such as C₃H₃F₂.

According to one embodiment, R⁴ is a 3-membered saturated carbocycle, which is substituted with halogen. More specifically by Cl, such as C₃H₃Cl₂.

According to one embodiment, R⁴ is a 4-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is a 5-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R⁴ is a 6-membered saturated carbocycle. According to one embodiment thereof, the carbocycle is unsubstituted, i.e. it does not carry any substituent R^{4b}. According to still another embodiment of formula I, it is substituted with R^{4b}.

According to still another embodiment of formula I, R⁴ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{4b} as defined below. According to one embodiment thereof, the heterocycle is unsubstituted.

According to still another embodiment of formula I, R⁴ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{4b} as defined below. According to one embodiment thereof, the heterocycle is unsubstituted.

According to still another embodiment of formula I, in the embodiments of R⁴ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one 0.

According to one embodiment, R⁴ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{4b}. According to still another embodiment of formula I, it is substituted with R^{4b}.

According to still another embodiment of formula I, R⁴ is a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{4b}. According to still another embodiment of formula I, it is substituted with R^{4b}.

According to still another embodiment of formula I, R⁴ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{4b}. According to still another embodiment of formula I, it is substituted with R^{4b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{4b}. According to still another embodiment of formula I, it is substituted with R^{4b}.

According to still another embodiment of formula I, R⁴ is phenyl-C₁-C₆-alkyl, such as phenyl-CH₂, wherein the phenyl moiety in each case is unsubstituted or substituted with one, two or three identical or different groups R^{4b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃ and S(O)₂CH₃.

According to still another embodiment of formula I, R⁴ is aryl, in particular phenyl, wherein the aryl or phenyl moiety in each case is unsubstituted or substituted with identical or different groups R^{4b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃. According to one embodiment, R⁴ is unsubstituted phenyl. According to another embodiment, R⁴ is phenyl, that is substituted with one, two or three, in particular one, halogen, in particular selected from F, Cl and Br, more specifically selected from F and Cl.

According to still another embodiment of formula I, R⁴ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁴ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further specific embodiment of formula I, R⁴ is C₁-C₆-alkyl, especially CH₂ substituted by a 5-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains one N as ring member. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains two N as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains three N as ring members. According to one embodiment thereof, the heteroaryl is unsubstituted, i.e. it does not carry any substituent R^{4b}. According to still another embodiment of formula I, it is substituted by R^{4b}. According to one specific embodiment thereof, said 5-membered saturated heterocycle contains 1 or 2, in particular 1, heteroatom(s) O.

According to a further specific embodiment of formula I, R⁴ is C₁-C₆-alkyl, especially CH₂ substituted by a 5-membered saturated heteroaryl which contains one S as ring member.

According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains one S and one N as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains one S and two N as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains one oxygen and one N as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 5-membered saturated heteroaryl which contains one oxygen and two N as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 6-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 6-membered saturated heteroaryl which one N as ring member. According to one embodiment thereof, the heteroaryl is unsubstituted, i.e. it does not carry any substituent R^{4b}. According to still another embodiment of formula I, it is substituted by R^{4b}.

According to a further specific embodiment of formula I, R⁴ is C₁-C₆-alkyl, especially CH₂ substituted by a 6-membered saturated heteroaryl which two N as ring members. According to one embodiment thereof, the heteroaryl is unsubstituted, i.e. it does not carry any substituent R^{4b}. According to still another embodiment of formula I, it is substituted by R^{4b}. According to a further specific embodiment of formula I, R⁴ is C₁-C₆-alkyl, especially CH₂ substituted by a 10-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{4b}.

According to still another embodiment of formula I, it is substituted by R^{4b}. According to one specific embodiment thereof, said 10-membered saturated heterocycle contains 1 or 2, in particular 1, heteroatom(s) N.

According to a further specific embodiment of formula I, R⁴ is C₁-C₆-alkyl, especially CH₂ substituted by a 10-membered saturated heteroaryl which one N as ring members. According to one embodiment thereof, the heteroaryl is unsubstituted, i.e. it does not carry any substituent R^{4b}. According to still another embodiment of formula I, it is substituted by R^{4b}.

According to still another embodiment of formula I, R⁴ is CH₂ substituted by a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁴ is CH₂ substituted by a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further particular embodiment, R⁴ is selected from C₁-C₆-alkyl, C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{4b} as defined below. According to one embodiment thereof, the carbocycle, heterocycle, heteroaryl and aryl are unsubstituted. In a particular embodiment, R⁴ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{4b} as defined below.

According to a further particular embodiment, R⁴ is selected from C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{4b} as defined below. According to one embodiment thereof, the carbocycle, heterocycle, heteroaryl and aryl are unsubstituted. In a particular embodiment, R⁴ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{4b} as defined below.

Particularly preferred embodiments of R⁴ according to the invention are in Table P4 below, wherein each line of lines P4-1 to P4-182 corresponds to one particular embodiment of the invention, wherein P4-1 to P4-182 are also in any combination with one another a preferred embodiment of the present invention. The connection point to the carbon atom, to which R⁴ is bound is marked with “#” in the drawings.

TABLE P4

| No. | R⁴ |
|---|---|
| P4-1 | CH₃ |
| P4-2 | CH₂CH₃ |
| P4-3 | CF₃ |
| P4-4 | CH₂F |
| P4-5 | CH₂Cl |
| P4-6 | CHF₂ |
| P4-7 | CHCl₂ |
| P4-8 | CH₂CF₃ |
| P4-9 | CH₂CCl₃ |
| P4-10 | CF₂CHF₂ |
| P4-11 | CH₂OCH₃ |
| P4-12 | CH₂OCH₂F |
| P4-13 | CH₂OCHF₂ |
| P4-14 | CH₂OCF₃ |
| P4-15 | CH₂OCF₂CHF₂ |
| P4-16 | CH₂NHMe |
| P4-17 | CH₂SMe |
| P4-18 | CH₂SOMe |
| P4-19 | CH₂SO₂Me |
| P4-20 | CH₂NMe₂ |
| P4-21 | CH₂NSO₂CF₃ |
| P4-22 | CH₂NSO₂CH₃ |
| P4-23 | CN |
| P4-24 | CH₂CN |
| P4-25 | CHO |
| P4-26 | COMe |
| P4-27 | CO₂Me |
| P4-28 | CH₂CHO |
| P4-29 | CH₂COMe |
| P4-30 | CH₂CO₂Me |
| P4-31 | 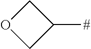 |
| P4-32 | 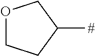 |
| P4-33 | 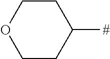 |
| P4-34 | 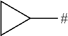 |
| P4-35 | 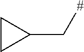 |
| P4-36 | 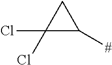 |
| P4-37 | 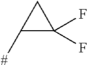 |
| P4-38 | 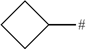 |
| P4-39 | 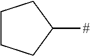 |
| P4-40 | 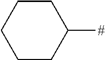 |
| P4-41 | 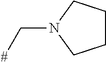 |
| P4-42 | 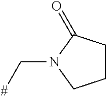 |
| P4-43 | 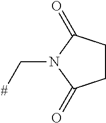 |
| P4-44 | 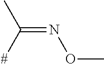 |
| P4-45 | 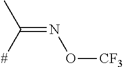 |
| P4-46 | 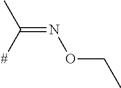 |
| P4-47 | 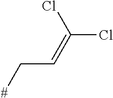 |
| P4-48 | 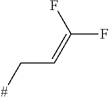 |
| P4-49 | 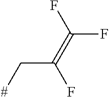 |
| P4-50 | 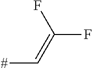 |
| P4-51 | 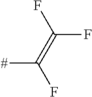 |
| P4-52 | 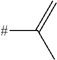 |
| P4-53 | 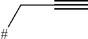 |
| P4-54 | 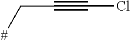 |
| P4-55 | 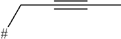 |
| P4-56 | 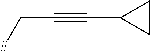 |
| P4-57 | 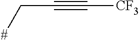 |
| P4-58 | 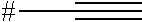 |
| P4-59 | 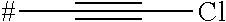 |
| P4-60 | 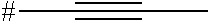 |
| P4-61 | 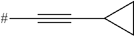 |
| P4-62 | 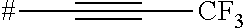 |
| P4-63 | C₆H₅ |
| P4-64 | 4-Cl—C₆H₄ |
| P4-65 | 3-Cl—C₆H₄ |
| P4-66 | 2-Cl—C₆H₄ |
| P4-67 | 2,4-Cl₂—C₆H₃ |
| P4-68 | 4-F—C₆H₄ |
| P4-69 | 3-F—C₆H₄ |
| P4-70 | 2-F—C₆H₄ |
| P4-71 | 2,4-F₂—C₆H₃ |
| P4-72 | 2,4-MeO—C₆H₄ |
| P4-73 | 3-MeO—C₆H₄ |
| P4-74 | 2-MeO—C₆H₄ |
| P4-75 | 4-MeO₂S—C₆H₄ |
| P4-76 | 3-MeO₂S—C₆H₄ |
| P4-77 | 2-MeO₂S—C₆H₄ |
| P4-78 | —CH₂—C₆H₅ |
| P4-79 | —CH₂—C₆H₄—4-F |
| P4-80 | —CH₂—C₆H₄—4-Cl |
| P4-81 | —CH₂—C₆H₃—2,4-Cl₂ |
| P4-82 | —CH₂—C₆H₄—4-SO₂Me |
| P4-83 | 3-py |
| P4-84 | 2-py |
| P4-85 | 4-py |
| P4-86 | 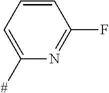 |
| P4-87 | 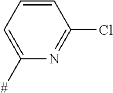 |
| P4-88 | 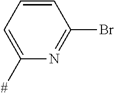 |
| P4-89 | 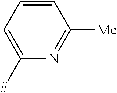 |
| P4-90 | 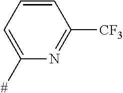 |
| P4-91 | 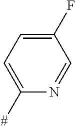 |
| P4-92 | 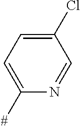 |
| P4-93 | 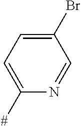 |
| P4-94 | 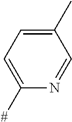 |
| P4-95 | 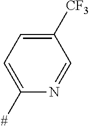 |
| P4-96 | 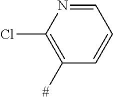 |
| P4-97 | 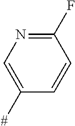 |
| P4-98 | 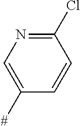 |
| P4-99 | 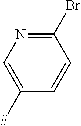 |
| P4-100 | 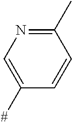 |
| P4-101 | 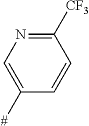 |
| P4-102 | 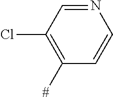 |
| P4-103 | 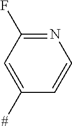 |
| P4-104 | 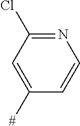 |
| P4-105 | 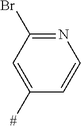 |
| P4-106 | 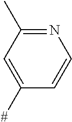 |
| P4-107 | 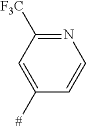 |
| P4-108 | 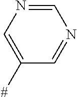 |
| P4-109 | 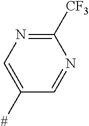 |
| P4-110 | 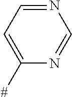 |
| P4-111 | 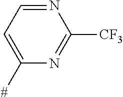 |
| P4-112 | 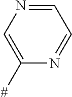 |
| P4-113 | 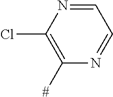 |
| P4-114 | 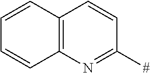 |
| P4-115 | 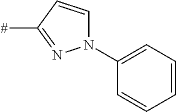 |
| P4-116 | 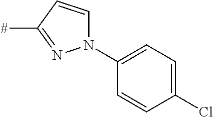 |
| P4-117 | 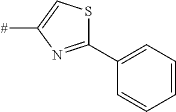 |
| P4-118 | 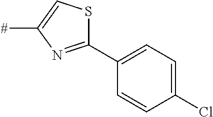 |
| P4-119 | 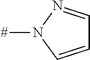 |
| P4-120 | 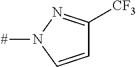 |
| P4-121 | 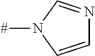 |
| P4-122 | 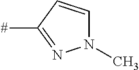 |
| P4-123 | 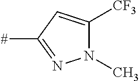 |
| P4-124 | 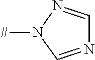 |
| P4-125 | 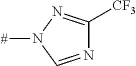 |
| P4-126 | 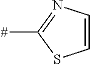 |
| P4-127 | 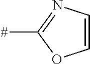 |
| P4-128 | 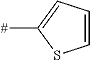 |
| P4-129 | 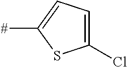 |
| P4-130 | 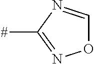 |
| P4-131 | 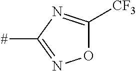 |
| P4-132 | 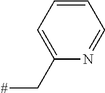 |
| P4-133 | 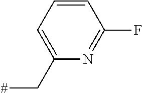 |
| P4-134 | 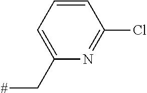 |
| P4-135 | 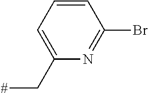 |
| P4-136 | 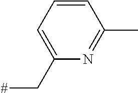 |
| P4-137 | 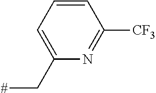 |
| P4-138 | 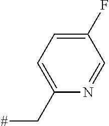 |
| P4-139 | 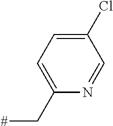 |
| P4-140 | 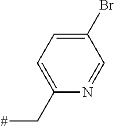 |
| P4-141 | 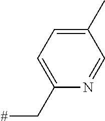 |
| P4-142 | 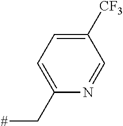 |
| P4-143 | 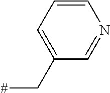 |
| P4-144 | 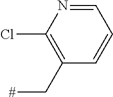 |
| P4-145 | 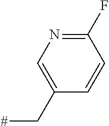 |
| P4-146 | 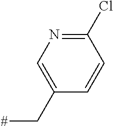 |
| P4-147 | 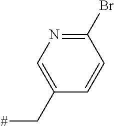 |
| P4-148 | 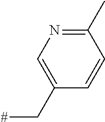 |
| P4-149 | 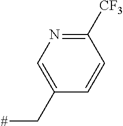 |
| P4-150 | 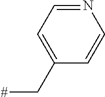 |
| P4-151 | 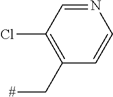 |
| P4-152 | 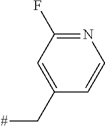 |
| P4-153 | 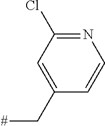 |
| P4-154 | 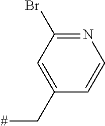 |
| P4-155 | 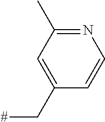 |
| P4-156 | 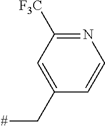 |
| P4-157 | 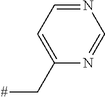 |
| P4-158 | 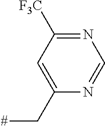 |
| P4-159 | 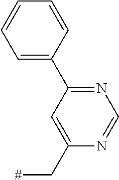 |
| P4-160 | 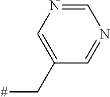 |
| P4-161 | 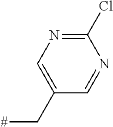 |
| P4-162 | 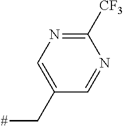 |
| P4-163 | 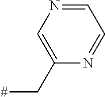 |
| P4-164 | 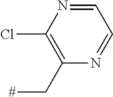 |
| P4-165 | 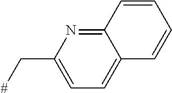 |
| P4-166 | 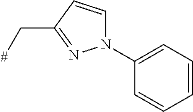 |
| P4-167 | 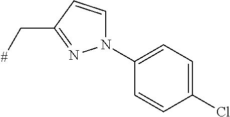 |
| P4-168 | 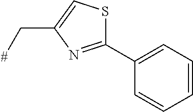 |
| P4-169 | 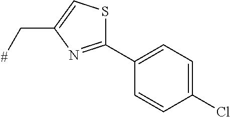 |
| P4-170 | 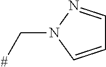 |
| P4-171 | 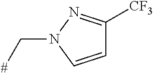 |
| P4-172 | 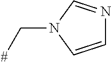 |
| P4-173 | 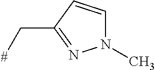 |
| P4-174 | 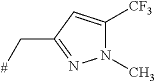 |
| P4-175 | 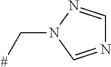 |
| P4-176 | 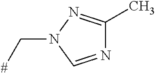 |
| P4-177 | 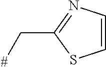 |
| P4-178 | 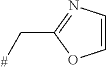 |
| P4-179 | 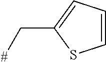 |
| P4-180 | 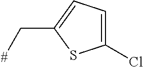 |
| P4-181 | 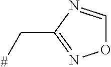 |
| P4-182 | 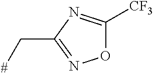 |

(py = pyridyl)

According to still another embodiment of formula I, R³, R⁴ together with the carbon atom to which they are bound form a saturated or partially unsaturated three-, four-, five-, six-, seven-, eight-, nine-, or ten-membered carbo- or heterocycle; wherein the heterocycle contains one, two, three or four heteroatoms selected from N, O and S, wherein the heteroatom N may carry one substituent R^{N} selected from C₁-C₄-alkyl, C₁-C₄-halogenalkyl and SO₂Ph, wherein Ph is unsubstituted phenyl or phenyl that is substituted with one, two or three substituents selected from CN, C₁-C₄-alkyl, halogen, C₁-C₄-halogenalkyl, C₁-C₄-alkoxy and C₁-C₄-halogenalkoxy; and wherein the heteroatom S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of CN, halogen, OH, C₁-C₄-alkyl, C₁-C₄-halogenalkyl, C₁-C₄-alkoxy, C₁-C₄-halogenalkoxy; and wherein in each case one or two CH₂ groups of the carbo- or heterocycle may be replaced by a group independently selected from C(═O) and C(═S).

According to one embodiment, R³ and R⁴ form a 3-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R³ and R⁴ form a 4-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R³ and R⁴ form a 5-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R³ and R⁴ form a 6-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R³ and R⁴ form a 7-membered saturated carbocycle. According to one embodiment thereof, the carbocycle is unsubstituted, i.e. it does not carry any substituent R^{4b}. According to still another embodiment of formula I, it is substituted with R^{4b}.

According to one embodiment, R³ and R⁴ together with the carbon atom to which they are bound form a saturated or partially unsaturated three-, four-, five-, six-, seven-, eight-, nine-, or ten-membered heterocycle that is unsubstituted or substituted. According to a further embodiment, the heterocycle formed by R³ and R⁴ is saturated.

According to a further embodiment, the heterocycle formed by R³ and R⁴ is a saturated unsubstituted or substituted heterocycle, wherein the heterocycle contains one, two or three, more particularly one or two, specifically one, heteroatom(s) selected from NH, NR^{N}, O, S, S(═O) and S(═O)₂, wherein RN is defined and preferably defined above. According to one embodiment, this saturated heterocycle is unsubstituted. According to a further embodiment, the saturated heterocycle carries one, two, three or four substituents R³⁴. In one further particular embodiment, said heterocycle is four- or six-membered.

According to a further embodiment, the unsubstituted or substituted and saturated or partially unsaturated heterocycle is three-, four-, five- or six-membered and contains one, two or three, more particularly one or two, heteroatoms selected from NH, NR^{N}, O, S, S(═O) and S(═O)₂, wherein R^{N} is as defined above or preferably selected from C₁-C₂-alkyl, C₁-C₂-halogenalkyl and SO₂Ph, wherein Ph is unsubstituted phenyl or phenyl that is substituted by one C₁-C₂-alkyl. In one further particular embodiment, said heterocycle is four- or six-membered.

According to a further embodiment, the heterocycle formed by R³ and R⁴ contains one, two or three, more specifically one or two, heteroatoms selected from NH and NR^{N}, wherein RN is as defined and preferably defined below, more particularly selected from C₁-C₂-alkyl, C₁-C₂-halogenalkyl and SO₂Ph, wherein Ph is unsubstituted phenyl or phenyl that is substituted by one methyl. In one embodiment thereof, it contains one or two heteroatoms NH, in particular one NH. In another embodiment, it contains one or two heteroatoms NR^{N}, in particular one NR^{N}, wherein R^{N} in each case is as defined and preferably defined above.

According to a further embodiment, the heterocycle formed by R³ and R⁴ contains one, two or three, more specifically one or two, in particular one, heteroatom(s) selected from S, S(═O) and S(═O)₂. In one embodiment thereof, it contains one or two heteroatoms S, in particular one S. In another embodiment, it contains one or two heteroatoms S(═O), in particular one S(═O). In still another embodiment, it contains one or two heteroatoms S(═O)₂, in particular one S(═O)₂.

According to a further embodiment, the heterocycle formed by R³ and R⁴ contains one or two heteroatoms O. In one embodiment thereof, it contains one heteroatom O. In another embodiment, it contains two heteroatoms O.

According to a further embodiment, the heterocycle formed by R³ and R⁴ is unsubstituted, i.e. it does not carry any substituent R³⁴. According to a further embodiment, it carries one, two, three or four R³⁴.

According to one particular embodiment, R³ and R⁴ together form a 4-membered saturated heterocycle which contains 1 or 2 heteroatoms, in particular 1 heteroatom, from the group consisting of NH, NR^{N}, O, S, S(═O) and S(═O)₂, as ring members, wherein R^{N} is defined and preferably defined above. In one embodiment, the heterocycle contains one O as heteroatom. For example, the formed heterocycle is oxetane. According to one embodiment thereof, the heterocycle is unsubstituted, i.e. it does not carry any substituent R³⁴. According to a further embodiment, it carries one, two, three or four R³⁴.

According to a further particular embodiment, R³ and R⁴ together form a 5-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined above. According to one embodiment thereof, the heterocycle is unsubstituted, i.e. it does not carry any substituent R³⁴. According to a further embodiment, it carries one, two, three or four R³⁴.

According to a further particular embodiment, R³ and R⁴ together form a 6-membered saturated heterocycle which contains 1, 2 or 3, in particular 1 or 2, heteroatoms from the group consisting of NH, NR^{N}, O, S, S(═O) and S(═O)₂, as ring members, wherein RN is as defined and preferably defined below. According to one embodiment thereof, the heterocycle is unsubstituted, i.e. it does not carry any substituent R³⁴. According to a further embodiment, it carries one, two, three or four R³⁴. According to one specific embodiment thereof, said 6-membered saturated heterocycle contains 1 or 2 heteroatoms selected from NH and NR^{N}. According to a further specific embodiment thereof, said 6-membered saturated heterocycle contains 1 or 2 heteroatoms O. According to a further specific embodiment thereof, said 6-membered saturated heterocycle contains 1 or 2 heteroatoms selected from S, S(═O) and S(═O)₂. According to one embodiment thereof, the respective 6-membered heterocycle is unsubstituted, i.e. it does not carry any substituent R³⁴. According to a further embodiment, it carries one, two, three or four R³⁴.

According to one further embodiment R³ together with R⁴ and with the carbon atom to which they are bound form a saturated three-, four-, five-, six-, seven-, eight-, nine-, or ten-membered carbocycle, in particular three-, four-, five- or six-membered carbocycle, more specifically five- or six-membered carbocycle, that is unsubstituted or carries one, two, three or four substituents R³⁴ as defined below. According to one embodiment thereof, R³ and R⁴ form a cyclopropyl, that is unsubstituted or carries one, two, three or four substituents R³⁴ as defined below. According to a further embodiment thereof, R³ and R⁴ form a cyclobutyl, that is unsubstituted or carries one, two, three or four substituents R³⁴ as defined below. According to still a further embodiment thereof, R³ and R⁴ form a cyclopentyl, that is unsubstituted or carries one, two, three or four substituents R³⁴ as defined below. According to still a further embodiment thereof, R³ and R⁴ form a cyclohexyl, that is unsubstituted or carries one, two, three or four substituents R³⁴ as defined below. According to still a further embodiment thereof, R³ and R⁴ form a cycloheptyl, that is unsubstituted or carries one, two, three or four substituents R³⁴ as defined below.

R³⁴ are the possible substituents for the carbo- or heterocycle formed by R³ and R⁴ and are independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S).

In one preferred embodiment, R³⁴ is in each case independently selected from halogen, OH, CN, SH, C₁-C₆-alkyl, C₁-C₆-halogenalkyl, C₁-C₆-alkoxy, C₁-C₆-halogenalkoxy and C₁-C₆-alkylthio. In one further preferred embodiment, R³⁴ is in each case independently selected from halogen, C₁-C₆-alkyl and C₁-C₆-halogenalkyl. In one further particular embodiment, R³⁴ is in each case independently selected from C₁-C₆-alkyl, such as methyl and ethyl.

R^{N} is the substituent of the heteroatom NR^{N} that is contained in the heterocycle formed by R³ and R⁴ in some of the inventive compounds. R^{N} is selected from C₁-C₄-alkyl, C₁-C₄-halogenalk and SO₂Ph, wherein Ph is unsubstituted phenyl or phenyl that is substituted by one, two or three substituents selected from C₁-C₄-alkyl. In one preferred embodiment, R^{N} is in each case independently selected from C₁-C₂-alkyl, C₁-C₂-halogenalkyl and SO₂Ph, wherein Ph is unsubstituted phenyl or phenyl that is substituted by one methyl substituents. In one particular embodiment, RN is in each case independently selected from C₁-C₂-alkyl, more particularly methyl. In one particular embodiment, RN is in each case independently selected from SO₂Ph, wherein Ph is unsubstituted phenyl or phenyl that is substituted by one methyl.

According to still another embodiment of formula I, R³, R⁴ together with the carbon atom to which they are bound form a saturated or partially unsaturated three-, four-, five-, six-, seven-, eight-, nine-, or ten-membered carbo- or heterocycle; wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of CN, halogen, OH, C₁-C₄-alkyl, C₁-C₄-halogenalkyl, C₁-C₄-alkoxy, C₁-C₄-halogenalkoxy.

According to still another embodiment of formula I, R³, R⁴ together with the carbon atom to which they are bound form a saturated or partially unsaturated four-, five-, six-membered carbo- or heterocycle; wherein the carbocycle or heterocycle is unsubstituted or carries one, two, three or four substituents R³⁴ independently selected from halogen, C₁-C₆-alkyl, C₁-C₆-halogenalkyl, C₁-C₆-alkoxy, C₁-C₆-halogenalkoxy.

Particularly preferred embodiments of combinations of R³ and R⁴ according to the invention are in Table P34 below, wherein each line of lines P34-1 to P34-190 corresponds to one particular embodiment of the invention, wherein P34-1 to P34-190 are also in any combination with one another a preferred embodiment of the present invention. The carbon atom, to which R³ and R⁴ are bound is marked with * in the drawings. “Ts” in the drawings stands for the tosyl group SO₂-(p-CH₃)phenyl.

TABLE P34

| No. | R³ | R⁴ |
|---|---|---|
| P34-1 | CH₃ | CH₃ |
| P34-2 | CH₃ | CH₂CH₃ |
| P34-3 | CH₃ | CF₃ |
| P34-4 | CH₃ | CHF₂ |
| P34-5 | CH₃ | CH₂F |
| P34-6 | CH₃ | CN |
| P34-7 | CH₃ | CH₂Cl |
| P34-8 | CH₃ | CH₂CH₂Cl |
| P34-9 | CH₃ | CH₂CH₂OCH₃ |
| P34-10 | CH₃ | 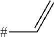 |
| P34-11 | CH₃ | 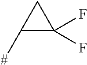 |
| P34-12 | CH₃ | 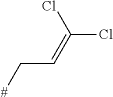 |
| P34-13 | CH₃ | 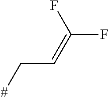 |
| P34-14 | CH₃ | 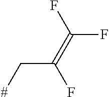 |
| P34-15 | CH₃ | 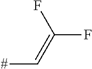 |
| P34-16 | CH₃ | 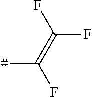 |
| P34-17 | CH₃ | 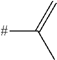 |
| P34-18 | CH₃ | 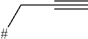 |
| P34-19 | CH₃ | 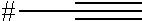 |
| P34-20 | CH₃ | C₆H₅ |
| P34-21 | CH₃ | 4-F—C₆H₄ |
| P34-22 | CH₃ | —CH₂—C₆H₅ |
| P34-23 | CH₃ | —CH₂—C₆H₄—4-F |
| P34-24 | CH₃ | 3-py |
| P34-25 | CH₃ | 2-py |
| P34-26 | CH₃ | 4-py |
| P34-27 | CH₃ | 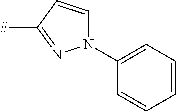 |
| P34-28 | CH₃ | 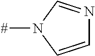 |
| P34-29 | CH₃ | 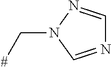 |
| P34-30 | CH₃ | 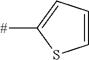 |
| P34-31 | CH₃ | 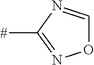 |
| P34-32 | CH₃ | 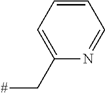 |
| P34-33 | CH₃ | 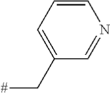 |
| P34-34 | CH₃ | 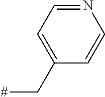 |
| P34-35 | CH₃ | 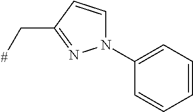 |
| P34-36 | CH₃ | 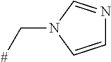 |
| P34-37 | CH₃ | 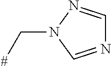 |
| P34-38 | CH₃ | 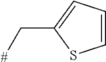 |
| P34-39 | CH₃ | 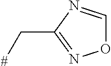 |
| P34-40 | CH₂F | CH₃ |
| P34-41 | CH₂F | CH₂CH₃ |
| P34-42 | CH₂F | CF₃ |
| P34-43 | CH₂F | CHF₂ |
| P34-44 | CH₂F | CH₂F |
| P34-45 | CH₂F | CN |
| P34-46 | CH₂F | CH₂Cl |
| P34-47 | CH₂F | CH₂CH₂Cl |
| P34-48 | CH₂F | CH₂CH₂OCH₃ |
| P34-49 | CH₂F | 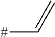 |
| P34-50 | CH₂F | 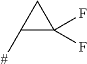 |
| P34-51 | CH₂F | 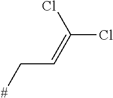 |
| P34-52 | CH₂F | 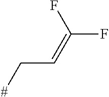 |
| P34-53 | CH₂F | 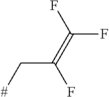 |
| P34-54 | CH₂F | 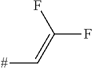 |
| P34-55 | CH₂F | 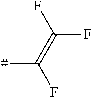 |
| P34-56 | CH₂F | 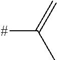 |
| P34-57 | CH₂F | 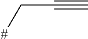 |
| P34-58 | CH₂F | 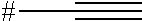 |
| P34-59 | CH₂F | C₆H₅ |
| P34-60 | CH₂F | 4-F—C₆H₄ |
| P34-61 | CH₂F | —CH₂—C₆H₅ |
| P34-62 | CH₂F | —CH₂—C₆H₄—4-F |
| P34-63 | CH₂F | 3-py |
| P34-64 | CH₂F | 2-py |
| P34-65 | CH₂F | 4-py |
| P34-66 | CH₂F | 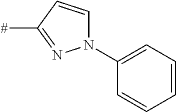 |
| P34-67 | CH₂F | 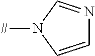 |
| P34-68 | CH₂F | 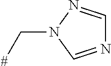 |
| P34-69 | CH₂F | 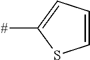 |
| P34-70 | CH₂F | 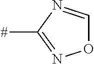 |
| P34-71 | CH₂F | 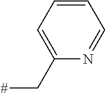 |
| P34-72 | CH₂F | 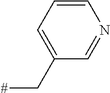 |
| P34-73 | CH₂F | 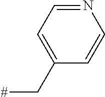 |
| P34-74 | CH₂F | 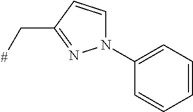 |
| P34-75 | CH₂F | 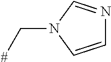 |
| P34-76 | CH₂F | 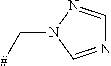 |
| P34-77 | CH₂F | 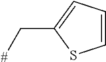 |
| P34-78 | CH₂F | 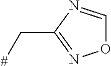 |
| P34-79 | CHF₂ | CH₃ |
| P34-80 | CHF₂ | CH₂CH₃ |
| P34-81 | CHF₂ | CF₃ |
| P34-82 | CHF₂ | CHF₂ |
| P34-83 | CHF₂ | CH₂F |
| P34-84 | CHF₂ | CN |
| P34-85 | CHF₂ | CH₂Cl |
| P34-86 | CHF₂ | CH₂CH₂Cl |
| P34-87 | CHF₂ | CH₂CH₂OCH₃ |
| P34-88 | CHF₂ | 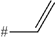 |
| P34-89 | CHF₂ | 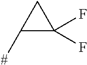 |
| P34-90 | CHF₂ | 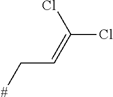 |
| P34-91 | CHF₂ | 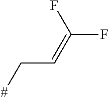 |
| P34-92 | CHF₂ | 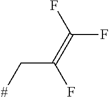 |
| P34-93 | CHF₂ | 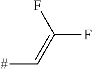 |
| P34-94 | CHF₂ | 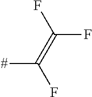 |
| P34-95 | CHF₂ | 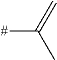 |
| P34-96 | CHF₂ | 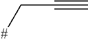 |
| P34-97 | CHF₂ | 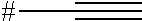 |
| P34-98 | CHF₂ | C₆H₅ |
| P34-99 | CHF₂ | 4-F—C₆H₄ |
| P34-100 | CHF₂ | —CH₂—C₆H₅ |
| P34-101 | CHF₂ | —CH₂—C₆H₄—4-F |
| P34-102 | CHF₂ | 3-py |
| P34-103 | CHF₂ | 2-py |
| P34-104 | CHF₂ | 4-py |
| P34-105 | CHF₂ | 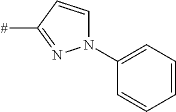 |
| P34-106 | CHF₂ | 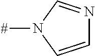 |
| P34-107 | CHF₂ | 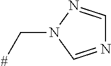 |
| P34-108 | CHF₂ | 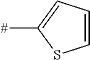 |
| P34-109 | CHF₂ | 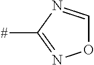 |
| P34-110 | CHF₂ | 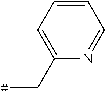 |
| P34-111 | CHF₂ | 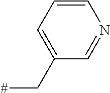 |
| P34-112 | CHF₂ | 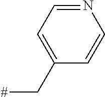 |
| P34-113 | CHF₂ | 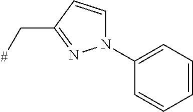 |
| P34-114 | CHF₂ | 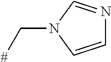 |
| P34-115 | CHF₂ | 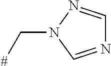 |
| P34-116 | CHF₂ | 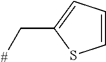 |
| P34-117 | CHF₂ | 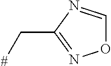 |
| P34-118 | CF₃ | CH₃ |
| P34-119 | CF₃ | CH₂CH₃ |
| P34-120 | CF₃ | CF₃ |
| P34-121 | CF₃ | CHF₂ |
| P34-122 | CF₃ | CH₂F |
| P34-123 | CF₃ | CN |
| P34-124 | CF₃ | CH₂Cl |
| P34-125 | CF₃ | CH₂CH₂Cl |
| P34-126 | CF₃ | CH₂CH₂OCH₃ |
| P34-127 | CF₃ | 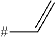 |
| P34-128 | CF₃ | 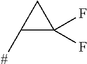 |
| P34-129 | CF₃ | 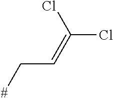 |
| P34-130 | CF₃ | 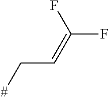 |
| P34-131 | CF₃ | 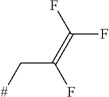 |
| P35-132 | CF₃ | 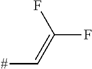 |
| P34-133 | CF₃ | 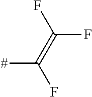 |
| P34-134 | CF₃ | 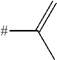 |
| P34-135 | CF₃ | 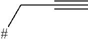 |
| P34-136 | CF₃ | 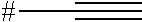 |
| P34-137 | CF₃ | C₆H₅ |
| P34-138 | CF₃ | 4-F—C₆H₄ |
| P34-139 | CF₃ | —CH₂—C₆H₅ |
| P34-140 | CF₃ | —CH₂—C₆H₄—4-F |
| P34-141 | CF₃ | 3-py |
| P34-142 | CF₃ | 2-py |
| P34-143 | CF₃ | 4-py |
| P34-144 | CF₃ | 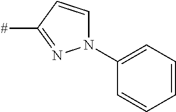 |
| P34-145 | CF₃ | 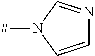 |
| P34-146 | CF₃ | 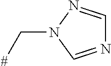 |
| P34-147 | CF₃ | 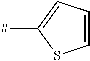 |
| P34-148 | CF₃ | 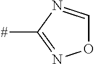 |
| P34-149 | CF₃ | 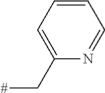 |
| P34-150 | CF₃ | 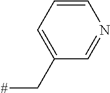 |
| P34-151 | CF₃ | 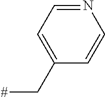 |
| P34-152 | CF₃ | 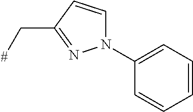 |
| P34-153 | CF₃ | 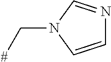 |
| P34-154 | CF₃ | 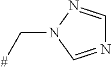 |
| P34-155 | CF₃ | 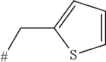 |
| P34-156 | CF₃ | 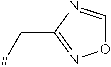 |

| | |
|---|---|
| P34-157 | 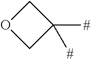 |
| P34-158 | 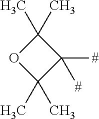 |
| P34-159 | 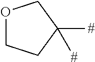 |
| P34-160 | 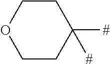 |
| P34-161 | 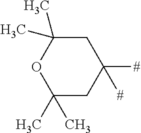 |
| P34-162 | 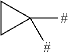 |
| P34-163 | 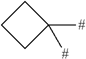 |
| P34-164 | 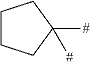 |
| P34-165 | 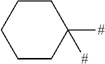 |
| P34-166 | 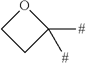 |
| P34-167 | 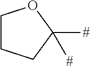 |
| P34-168 | 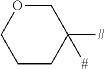 |
| P34-169 | 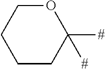 |
| P34-170 | 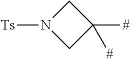 |
| P34-171 | 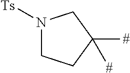 |
| P34-172 | 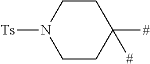 |
| P34-173 | 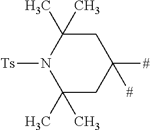 |
| P34-174 | 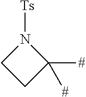 |
| P34-175 | 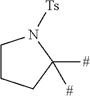 |
| P34-176 | 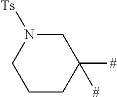 |
| P34-177 | 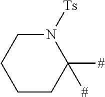 |
| P34-178 | 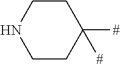 |
| P34-179 | 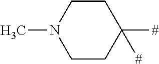 |
| P34-180 | 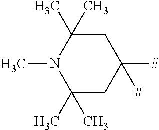 |
| P34-181 | 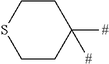 |
| P34-182 | 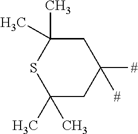 |
| P34-183 | 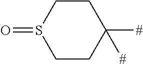 |
| P34-184 | 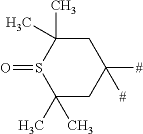 |
| P34-185 | 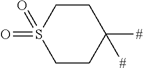 |
| P34-186 | 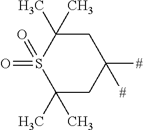 |
| P34-187 | 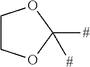 |
| P34-188 | 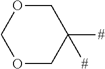 |
| P34-189 | 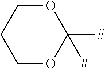 |
| P34-190 | 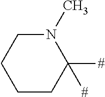 |

R^{x} in the substituent NH—SO₂—R^{x} is in each case independently selected from C₁-C₄-alkyl, C₁-C₄-halogenalkyl, unsubstituted aryl and aryl that is substituted by one, two, three, four or five substituents R^{x1} independently selected from C₁-C₄-alkyl, halogen, OH, CN, C₁-C₄-halogenalkyl, C₁-C₄-alkoxy and C₁-C₄-halogenalkoxy. In particular, R^{x} is in each case independently selected from C₁-C₄-alkyl and phenyl that is substituted by one, two or three R^{x1} independently selected from C₁-C₂-alkyl, more specifically R^{x} is in each case independently selected from C₁-C₄-alkyl and phenyl that is substituted by one CH₃, more specifically SO₂—R^{x} is the tosyl group (“Ts”).

R^{4a} are the possible substituents for the the acyclic moieties of R⁴ and the R^{4a} are in each case independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), CR′═NOR″, a saturated or partially unsaturated three-, four-, five-, six-, seven-, eight-, nine-, or ten-membered carbocycle or heterocycle, five-, six- or ten-membered heteroaryl, aryl, phenoxy; wherein in each case one or two CH₂ groups of the carbocycle and heterocycle may be replaced by a group independently selected from C(═O) and C(═S); wherein the heterocycle and heteroaryl contain independently one, two, three or four heteroatoms selected from N, O and S; wherein the carbocyclic, heterocyclic, phenyl and heteroaryl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy and S(O)ₙ—C₁-C₆-alkyl; wherein n is 0, 1 and 2;

According to one preferred embodiment, R^{4a} is in each case independently selected from halogen, OH, CN, NH(C₁-C₄-alkyl), N(C₁-C₄-alkyl)₂, NH—SO₂—R^{x}, CH(═O), C(═O)C₁-C₆-alkyl, C(═O)O(C₁-C₆-alkyl), C(═O)NH(C₁-C₆-alkyl) and CR′═NOR″.

According to one preferred embodiment, R^{4a} is in each case independently selected from OH, CN, CH(═O), C(═O)C₁-C₆-alkyl, C(═O)O(C₁-C₆-alkyl), C(═O)NH(C₁-C₆-alkyl) such as CN, CHO, C(O)O(CH₃),CO₂NH(CH₃), CO₂N(CH₃)₂ or NHSO₂CF₃.

According to one preferred embodiment, R^{4a} is in each case independently selected from C₁-C₆-alkylthio, C₁-C₆-halogenalkylthio, S(O)ₙ—C₁-C₆-alkyl, S(O)ₙ-aryl, such as SCH₃, SO₂CH₃, SO₂Ph.

According to one preferred embodiment, R^{4a} is in each case independently selected from NH(C₁-C₄-alkyl), N(C₁-C₄-alkyl)₂, NH—SO₂—R^{x}, such as NH(CH₃), N(CH₃)₂ or NHSO₂CH₃, NHSO₂CF₃.

According to one preferred embodiment, R^{4a} is in each case independently selected from C₃-C₆-cycloalkyl, C₃-C₆-halogencycloalkyl, such as cyclopropyl or fully or partially halogenated cyclopropyl.

According to one preferred embodiment, R^{4a} is in each case independently selected from C₁-C₆-alkoxy, C₁-C₆-halogenalkoxy, such as OCF₃, OCHF₂, OCH₂F, OCCl₃, OCHCl₂ or OCH₂Cl, in particular OCF₃, OCHF₂, OCCl₃ or OCHCl₂.

According to one preferred embodiment, R^{4a} is in each case independently selected from heterocarbocycle, wherein the heretocyclocycle is a saturated, two CH₂ groups are replaced by C(═O) and contains one N as a ring member.

According to one preferred embodiment, R^{4a} is in each case independently selected from aryl, wherein the aryl is substituted with halogen selected from the group consisting of F, Cl, Br, CH₃, CHF₂, OCH₃, OCHF₃, CN or SO₂CH₃.

According to one prefer embodiment, R⁴ is unsubstituted 5- or 6-membered heteroaryl. According to still a further embodiment, R⁴ is 5- or 6-membered heteroaryl substituted by halogen selected from the group consisting of F, Cl, Br, CH₃, CHF₂, OCH₃, OCHF₃, CN or SO₂CH₃.

According to one preferred embodiment, R^{4a} is in each case independently selected from halogen, OH, CN, C₃-C₆-cycloalkyl, C₃-C₆-halogencycloalkyl and heterocycle, wherein the heretocyclocycle is a saturated and contains one N as a ring member.

According to one preferred embodiment, R^{4a} is in each case independently selected from halogen, OH, CN, C₃-C₆-cycloalkyl, C₃-C₆-halogencycloalkyl and heterocycle, wherein the heretocyclocycle is a saturated, one CH₂ group is replaced by C(═O) and contains one N as a ring member.

According to one preferred embodiment, R^{4a} is in each case independently selected from halogen, OH, CN, C₃-C₆-cycloalkyl, C₃-C₆-halogencycloalkyl and heterocycle, wherein the heretocyclocycle is a saturated, two CH₂ groups are replaced by C(═O) and contains one N as a ring member.

According to one preferred embodiment, R^{4a} is in each case independently selected from halogen, CN, C₁-C₆-alkoxy, C₁-C₆-halogenalkoxy, C₃-C₇-cycloalkyl, C₃-C₇-halogencycloalkyl, aryl, and heteroaryl, wherein the aryl and heteroaryl are substituted from the group consisting of F, Cl, Br, CH₃, CHF₂, OCH₃, OCHF₃, CN or SO₂CH₃. According to one further preferred embodiment, R^{4a} is in each case independently selected from halogen, phenyl, halogenphenyl and heteroaryl, wherein the halogenphenyl is substituted with halogen selected from the group consisting of F, Cl and Br, in particular selected from F and Cl.

According to one preferred embodiment, R^{4a} is in each case independently selected from halogen, CN, C₁-C₆-alkoxy, C₁-C₆-halogenalkoxy, aryl, and heteroaryl, wherein the aryl and heteroaryl are substituted from the group consisting of F, Cl, Br, CH₃, CHF₂, OCH₃, OCHF₃, CN or SO₂CH₃. According to one further preferred embodiment, R^{4a} is in each case independently selected from halogen, phenyl, halogenphenyl and heteroaryl, wherein the halogenphenyl is substituted with halogen selected from the group consisting of F, Cl and Br, in particular selected from F and Cl.

According to one further preferred embodiment, R^{4a} is in each case independently selected from halogen, CN, C₁-C₆-alkoxy and phenyl; wherein the phenyl is substituted with halogen selected from the group consisting of F, Cl and Br or by C₁-C₄-alkyl, C₁-C₄-halogenalkyl, C₁-C₄-alkoxy and C₁-C₄-halogenalkoxy. According to one further preferred embodiment, R^{4a} is in each case independently selected from CN, halogen, C₁-C₆-alkoxy, phenyl and heteroaryl, wherein the phenyl and heteroaryl is substituted with halogen selected from the group consisting of F, Cl and Br, in particular selected from F and Cl.

R^{4b} are the possible substituents for the carbocycle, heterocycle, heteroaryl and aryl moieties of R⁴ and are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy.

According to one preferred embodiment, R^{4b} is in each case independently selected from halogen, OH, CN, SH, C₁-C₆-alkyl, C₁-C₆-halogenalkyl, C₁-C₆-alkoxy, C₁-C₆-halogenalkoxy, C₁-C₆-alkylthio and S(O)ₙ—C₁-C₆-alkyl. According to one further preferred embodiment, R^{4b} is in each case independently selected from halogen, C₁-C₆-alkoxy, C₁-C₆-halogenalkyl, C₁-C₆-halogenalkoxy and S(O)ₙ—C₁-C₆-alkyl. According to one further particular embodiment, R^{4b} is in each case independently selected from C₁-C₆-alkyl, such as methyl and ethyl. According to one further particular embodiment, R^{4b} is in each case independently selected from halogen, such as F, Cl and Br. According to one further particular embodiment, R^{4b} is in each case independently selected from C₁-C₆-alkoxy, such as OCH₃. According to one further particular embodiment, R^{4b} is in each case independently selected from C₁-C₄-halogenalkoxy, such as OCHF₂ and OCF₃. According to one further particular embodiment, R^{4b} is in each case independently selected from S(O)ₙ—C₁-C₆-alkyl. such as SO₂CH₃.

### R⁵ is H.

### R⁶ is H.

R⁷ and R⁸ together with the carbon atoms to which they are bound together form a phenyl or five- or six-membered heteroaryl; wherein the heteroaryl contains one, two or three heteroatoms selected from N, O and S; and wherein the heteroaryl carries zero, one, two, three or four substituents (R⁷⁸)ₒ, wherein o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)ₙ—C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R^{x}, R′ and R″ are as defined above.
and wherein the acyclic moieties of R⁷⁸ are not further substituted or carry one, two, three or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl and phenyl group is unsubstituted or carries one, two, three, four or five substituents R^{78aa} selected from the group consisting of halogen, OH, C₁-C₄-alkyl, C₁-C₄-halogenalkyl, C₁-C₄-alkoxy and C₁-C₄-halogenalkoxy;
wherein the carbocyclic, phenyl, heterocyclic and heteroaryl moieties of R⁷⁸ are not further substituted or carry one, two, three, four, five or up to the maximum number of identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment, R⁷ and R⁸ together with the carbon atoms to which they are bound form phenyl; wherein the phenyl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to one embodiment, R⁷ and R⁸ together with the carbon atoms to which they are bound form a five- or six-membered heteroaryl; wherein the heteroaryl contains one or two heteroatoms selected from N, O and S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a five- or six-membered heteroaryl; wherein the heteroaryl contains one or two heteroatoms N, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a five- or six-membered heteroaryl; wherein the heteroaryl contains one or two heteroatoms selected from S and O, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a five- or six-membered heteroaryl; wherein the heteroaryl contains one heteroatom S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a five- or six-membered heteroaryl; wherein the heteroaryl contains one heteroatom O, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to one embodiment, R⁷ and R⁸ together with the carbon atoms to which they are bound form a five-membered heteroaryl; wherein the heteroaryl contains one or two heteroatoms selected from N, O and S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to one embodiment, R⁷ and R⁸ together with the carbon atoms to which they are bound form a five-membered heteroaryl; wherein the heteroaryl contains one or two heteroatoms N, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to one embodiment, R⁷ and R⁸ together with the carbon atoms to which they are bound form a five-membered heteroaryl; wherein the heteroaryl contains one or two heteroatoms selected from O and S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to one embodiment, R⁷ and R⁸ together with the carbon atoms to which they are bound form a five-membered heteroaryl; wherein the heteroaryl contains one heteroatom S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2.

According to one embodiment, R⁷ and R⁸ together with the carbon atoms to which they are bound form a five-membered heteroaryl; wherein the heteroaryl contains one heteroatom 0, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a six-membered heteroaryl; wherein the heteroaryl contains one or two heteroatoms selected from N, O and S, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to a further embodiment, R⁷ and R⁸ together with the carbon atoms to which they are bound form a six-membered heteroaryl; wherein the heteroaryl contains one or two heteroatoms N, and wherein the heteroaryl carries zero, one or two substituents (R⁷⁸)ₒ, as defined and preferably defined herein, wherein o is 0, 1 or 2. According to one specific embodiment, o is 0. According to a further embodiment, o is 1 or 2. Particular embodiments thereof are listed in Table P78.

According to the invention, there can be zero, one, two or three R⁷⁸ present, namely for o is 0, 1, 2 or 3.

According to one embodiment, o is 0.

According to a further embodiment, o is 1.

According to a further embodiment, o is 2 or 3. According to one specific embodiment thereof, o is 2, according to a further specific embodiment, o is 3.

For every R⁷⁸ that is present in the inventive compounds, the following embodiments and preferences apply independently of the meaning of any other R⁷⁸ that may be present in the ring. Furthermore, the particular embodiments and preferences given herein for R⁷⁸ apply independently for each of o=1, o=2 and o=3.

According to one specific embodiment, R⁷⁸ is halogen, in particular F, Cl, Br or I, more specifically F, Cl or Br, in particular F or Cl.

According to still another embodiment of formula I, R⁷⁸ is F.

According to still another embodiment of formula I, R⁷⁸ is Cl.

According to still another embodiment of formula I, R⁷⁸ is Br.

According to a further specific embodiment, R⁷⁸ is OH.

According to a further specific embodiment, R⁷⁸ is CN.

According to a further specific embodiment, R⁷⁸ is NO₂.

According to still another embodiment of formula I, R⁷⁸ is SH.

According to still another embodiment of formula I, R⁷⁸ is NH₂.

According to still another embodiment of formula I, R⁷⁸ is, NH(C₁-C₄-alkyl), N(C₁-C₄-alkyl)₂, NH(C(═O)(C₁-C₄-alkyl), N(C(═O)(C₁-C₄-alkyl)₂, wherein C₁-C₄-alkyl is CH₃, C₂H₅, n-propyl, i-propyl, n-butyl, i-butyl, tert-butyl, n-pentyl or i-pentyl.

According to a further specific embodiment of formula I, R⁷⁸ is NH—SO₂—R^{x} such as NH—SO₂—CH₃, NH—SO₂—CH₂—CH₃, NH—SO₂—CF₃ or NH—SO₂-Ts.

According to a further specific embodiment of formula I, R⁷⁸ is CH(═O), C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R⁷⁸ is CR′═NOR″ such as C(CH₃)═NOCH₃, C(CH₃)═NOCH₂CH₃ or C(CH₃)═NOCF₃.

According to a further specific embodiment, R⁷⁸ is C₁-C₆-alkyl, in particular C₁-C₄-alkyl, such as CH₃, C₂H₅, n-propyl, i-propyl, n-butyl, i-butyl, tert-butyl, n-pentyl or i-pentyl, in particular CH₃.

According to a further specific embodiment, R⁷⁸ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂. According to still a further embodiment, R⁷⁸ is C₂-C₆-alkenyl, in particular C₂-C₄-alkenyl, such as CH═CH₂ or CH₂ CH═CH₂.

According to still another embodiment of formula I R⁷⁸ is C₃-C₆-cycloalkyl, in particular cyclopropyl.

According to still another embodiment of formula I, R⁷⁸ is C₃-C₆-halogencycloalkyl. In a special embodiment R¹ is fully or partially halogenated cyclopropyl.

According to still a further embodiment, R⁷⁸ is C₃-C₆-cycloalkyl-C₂-C₆-alkenyl, in particular C₃-C₆-cycloalkyl-C₂-C₄-alkenyl, more specifically C₃-C₆-cycloalkyl-C₂-C₃-alkenyl, such as C₃H₅—CH═CH₂.

According to a further specific embodiment, R⁷⁸ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂. CH₂CF═CF₂, CH₂CCl═CCl₂. CF₂CF═CF₂ or CCl₂CCl═CCl₂.

According to still a further embodiment, R⁷⁸ is C₂-C₆-alkynyl, in particular C₂-C₄-alkynyl, more specifically C₂-C₃-alkynyl, such as C═CH.

According to still a further embodiment, R⁷⁸ is C₂-C₆-halogenalkynyl, in particular C₂-C₄-halogenalkynyl, more specifically C₂-C₃-halogenalkynyl.

According to a further specific embodiment, R⁷⁸ is C₁-C₆-alkoxy, in particular C₁-C₄-alkoxy, more specifically C₁-C₂-alkoxy such as OCH₃ or OCH₂CH₃.

According to a further specific embodiment, R⁷⁸ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂, OCH₂Cl and OCF₂CHF₂, in particular OCF₃, OCHF₂ and OCF₂CHF₂.

According to a further specific embodiment of formula I, R⁷⁸ is C₂-C₆-alkenyloxy, in particular C₂-C₄-alkenyloxy, more specifically C₁-C₂-alkenyloxy such as OCH═CH₂, OCH₂CH═CH₂.

According to a further specific embodiment of formula I, R⁷⁸ is C₂-C₆-alkynyloxy, in particular C₂-C₄-alkynyloxy, more specifically C₁-C₂-alkynyloxy such as OC≡CH

According to a further specific embodiment of formula I, R⁷⁸ is S(O)ₙ—C₁-C₆-alkyl, wherein alkyl is CH₃, C₂H₅, n-propyl, i-propyl, n-butyl, i-butyl, tert-butyl, n-pentyl or i-pentyl and n is 1, 2 or 3.

According to a further specific embodiment of formula I, R⁷⁸ is S(O)ₙ—C₁-C₆-halogenalkyl, wherein halogenalkyl is CF₃ or CHF₂ and n is 1, 2 or 3.

According to still another embodiment of formula I, R⁷⁸ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{78b} as defined below. According to one embodiment thereof, the heterocycle is unsubstituted.

According to still another embodiment of formula I, R⁷⁸ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{78b} as defined below. According to one embodiment thereof, the heterocycle is unsubstituted.

According to still another embodiment of formula I, in the embodiments of R⁷⁸ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one O.

According to one embodiment, R⁷⁸ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{78b}. According to still another embodiment of formula I, it is substituted by R^{78b}.

According to still another embodiment of formula I, R⁷⁸ is a 5-membered saturated heterocycle which contains 1, 2 or 3, in particular 1 or 2, heteroatoms from the group consisting of N, O and S, as ring members. According to one embodiment, the heterocycle contains one O as heteroatom.

According to one embodiment thereof, the heterocycle is unsubstituted, i.e. it does not carry any substituent R^{78b}. According to still another embodiment of formula I, it is substituted by R^{78b}.

According to still another embodiment of formula I, R⁷⁸ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{78b}. According to still another embodiment of formula I, it is substituted by R^{78b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{78b}. According to still another embodiment of formula I, it is substituted by R^{78b}.

According to still another embodiment of formula I, R⁷⁸ is phenyl-C₁-C₆-alkyl, such as phenyl-CH₂, wherein the phenyl moiety in each case is unsubstituted or substituted by one, two or three identical or different groups R^{78b} which independently of one another are selected from halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, CF₃ OCHF₂, and OCF₃.

According to still a further specific embodiment, R⁷⁸ is unsubstituted phenyl or phenyl that is substituted by one, two, three or four R^{78b}, as defined and preferably herein. In particular, R⁷⁸ is unsubstituted phenyl or phenyl that is substituted by one, two, three or four R^{78b}, as defined herein. In one embodiment R⁷⁸ is unsubstituted phenyl.

According to still another embodiment of formula I, R⁷⁸ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁷⁸ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to one further embodiment, R⁷⁸ is in each case independently selected from halogen, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy, C₃-C₆-cycloalkyl, S(O)ₙ—C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; and wherein the acyclic moieties of R⁷⁸ are not further substituted or carry one, two, three or up to the maximum possible number of identical or different groups R^{78a} as defined and preferably defined herein, and wherein the heterocyclic, alicyclic, phenyl and heteroaryl moieties of R⁷⁸ are not further substituted or carry one, two, three, four, five or up to the maximum number of identical or different groups R^{78b} as defined and preferably defined herein.

According to one further embodiment, R⁷⁸ is in each case independently selected from halogen, CN, C₁-C₆-alkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl, S(O)ₙ—C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; and wherein the acyclic moieties of R⁷⁸ are not further substituted or carry one, two, three or up to the maximum possible number of identical or different groups R^{78a} as defined and preferably defined herein, and wherein the heterocyclic, alicyclic, phenyl and heteroaryl moieties of R⁷⁸ are not further substituted or carry one, two, three, four, five or up to the maximum number of identical or different groups R^{78b} as defined and preferably defined herein. According to one specific embodiment, the acyclic and cyclic moieties of R⁷⁸ are not further substituted, according to another embodiment, the acyclic moieties of R⁷⁸ carry one, two, three or four identical or different groups R^{78a} as defined and preferably defined herein.

According to a further embodiment, R⁷⁸ is in each case independently selected from halogen, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy, C₃-C₆-cycloalkyl and S(O)ₙ—C₁-C₆-alkyl, wherein the acyclic moieties of R⁷⁸ are not further substituted or carry one, two, three or up to the maximum possible number of identical or different groups R^{78a} as defined and preferably defined herein, and wherein the cycloalkyl moieties of R⁷⁸ are not further substituted or carry one, two, three, four, five or up to the maximum number of identical or different groups R^{78b} as defined and preferably defined herein.

According to a further embodiment, R⁷⁸ is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl and S(O)ₙ—C₁-C₆-alkyl, wherein the acyclic moieties of R⁷⁸ are not further substituted or carry one, two, three or up to the maximum possible number of identical or different groups R^{78a} as defined and preferably defined herein, and wherein the cycloalkyl moieties of R⁷⁸ are not further substituted or carry one, two, three, four, five or up to the maximum number of identical or different groups R^{78b} as defined and preferably defined herein. According to one specific embodiment, the acyclic and cyclic moieties of R⁷⁸ are not further substituted, according to another embodiment, the acyclic moieties of R⁷⁸ carry one, two, three or four identical or different groups R^{78a} as defined and preferably defined herein.

According to still a further embodiment, R⁷⁸ is in each case independently selected from halogen, C₁-C₆-alkyl and C₁-C₆-alkoxy, wherein the acyclic moieties of R⁷⁸ are not further substituted or carry one, two, three or up to the maximum possible number of identical or different groups R^{78a} defined and preferably defined herein.

According to still a further embodiment, R⁷⁸ is in each case independently selected from CN, halogen, C₁-C₆-alkyl, C₁-C₆-halogenalkyl, C₁-C₆-alkoxy and C₁-C₆-halogenalkoxy, wherein the acyclic moieties of R⁷⁸ are not further substituted or carry one, two, three or up to the maximum possible number of identical or different groups R^{78a} defined and preferably defined herein. According to one specific embodiment, the acyclic and cyclic moieties of R⁷⁸ are not further substituted, according to another embodiment, the acyclic moieties of R⁷⁸ carry one, two, three or four identical or different groups R^{78a} as defined and preferably defined herein.

According to still a further embodiment, R⁷⁸ is in each case independently selected from halogen, C₁-C₆-alkyl, C₁-C₆-halogenalkyl, C₁-C₆-alkoxy, C₁-C₆-halogenalkoxy or CN.

R^{78a} are the possible substituents for the acyclic moieties of R⁷⁸. R^{78a} is independently selected from halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl and phenyl group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy.

According to one embodiment R^{78a} is independently selected from halogen, C₁-C₆-alkoxy, C₃-C₆-cycloalkyl, C₃-C₆-halogencycloalkyl and C₁-C₄-halogenalkoxy. Specifically, R^{78a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to a further embodiment, R^{78a} is independently halogen, in particular selected from F, Cl, Br and I, more specifically F, Cl and Br.

R^{78b} are the possible substituents for the cycloalkyl, heterocycle, heteroaryl and phenyl moieties of R⁷⁸. R^{78b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{78b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{78b} is independently selected from F, Cl, CN, CH₃, OCH₃ and halogenmethoxy.

Particularly preferred embodiments of R⁷ and R⁸, optionally substituted by (R⁷⁸)ₒ, according to the invention are in Table P78 below, wherein each line of lines P78-1 to P78-82 corresponds to one particular embodiment of the invention, wherein P78-1 to P78-82 are also in any combination with one another a preferred embodiment of the present invention. Thereby, the positions of the heteroaryls marked with “#” represents the connection points (carbon atoms 5′ and 6′ in formula I) with the remaining skeleton of the compounds of formula I:

TABLE P78

| “No. | R⁷ + R⁸ |
|---|---|
| P78-1 | 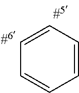 |
| P78-2 | 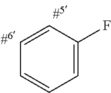 |
| P78-3 | 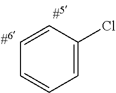 |
| P78-4 | 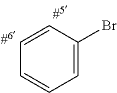 |
| P78-5 | 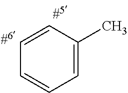 |
| P78-6 | 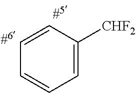 |
| P78-7 | 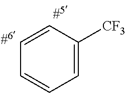 |
| P78-8 | 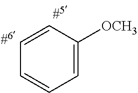 |
| P78-9 | 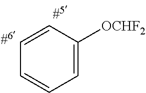 |
| P78-10 | 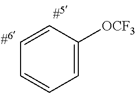 [SMILES: FC(F)(F)Oc1ccccc1] |
| P78-11 | 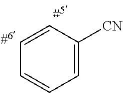 |
| P78-12 | 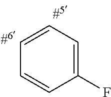 |
| P78-13 | 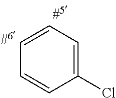 |
| P78-14 | 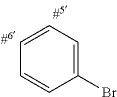 |
| P78-15 | 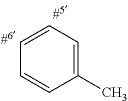 |
| P78-16 | 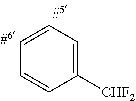 |
| P78-17 | 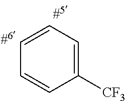 |
| P78-18 | 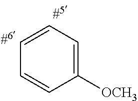 |
| P78-19 | 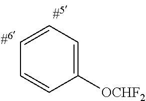 |
| P78-20 | 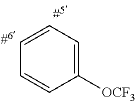 [SMILES: FC(F)(F)Oc1ccccc1] |
| P78-21 | 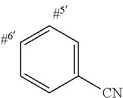 |
| P78-22 | 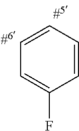 |
| P78-23 | 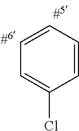 |
| P78-24 | 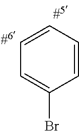 |
| P78-25 | 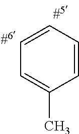 |
| P78-26 | 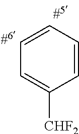 |
| P78-27 | 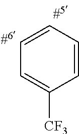 |
| P78-28 | 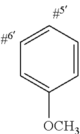 |
| P78-29 | 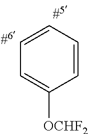 |
| P78-30 | 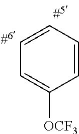 [SMILES: FC(F)(F)Oc1ccccc1] |
| P78-31 | 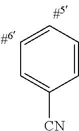 |
| P78-32 | 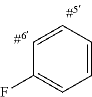 |
| P78-33 | 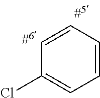 |
| P78-34 | 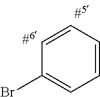 |
| P78-35 | 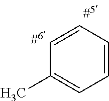 |
| P78-36 | 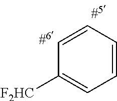 |
| P78-37 | 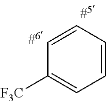 |
| P78-38 | 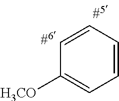 |
| P78-39 | 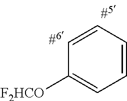 |
| P78-40 | 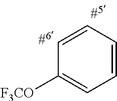 [SMILES: FC(F)(F)Oc1ccccc1] |
| P78-41 | 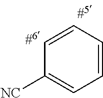 |
| P78-42 | 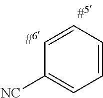 |
| P78-43 | 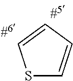 |
| P78-44 | 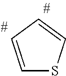 |
| P78-45 | 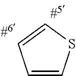 |
| P78-46 | 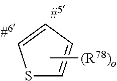 |
| P78-47 | 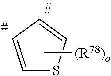 |
| P78-48 | 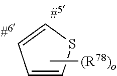 |
| P78-49 | 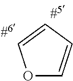 |
| P78-50 | 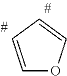 |
| P78-51 | 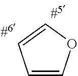 |
| P78-52 | 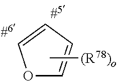 |
| P78-53 | 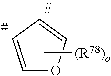 |
| P78-54 | 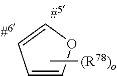 |
| P78-55 | 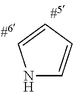 |
| P78-56 | 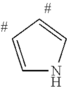 |
| P78-57 | 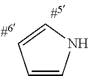 |
| P78-58 | 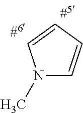 |
| P78-59 | 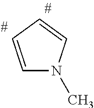 |
| P78-60 | 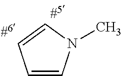 |
| P78-61 | 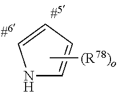 |
| P78-62 | 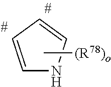 |
| P78-63 | 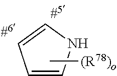 |
| P78-64 | 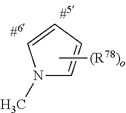 |
| P78-65 | 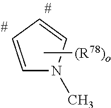 |
| P78-66 | 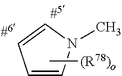 |
| P78-67 | 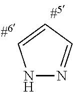 |
| P78-68 | 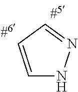 |
| P78-69 | 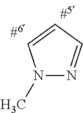 |
| P78-70 | 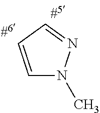 |
| P78-71 | 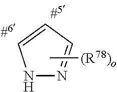 |
| P78-72 | 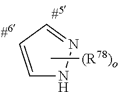 |
| P78-73 | 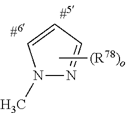 |
| P78-74 | 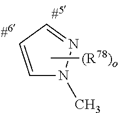 |
| P78-75 | 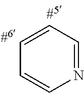 |
| P78-76 | 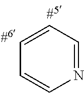 |
| P78-77 | 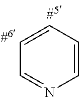 |
| P78-78 | 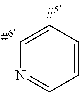 |
| P78-79 | 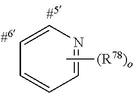 |
| P78-80 | 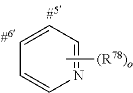 |
| P78-81 | 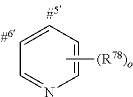 |
| P78-82 | 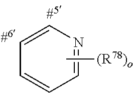 |

R⁹ is in each case independently selected from H, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₂-C₄-alkyl)(C₂-C₄-alkenyl), N(C₂-C₄-alkyl)(C₂-C₄-alkynyl), N(C₂-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, (═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl; phenyl and phenyl-C₁-C₆-alkyl; wherein the phenyl group is unsubstituted or substituted with substituents selected from the group consisting of halogen, CN, OH, C₁-C₆-alkyl, C₁-C₆-halogenalkyl, C₁-C₆-alkoxy and C₁-C₆-halogenalkoxy;
wherein the acyclic moieties of R⁹ are unsubstituted or substituted with groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenoxy group is unsubstituted or substituted with substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocycle, heteroaryl and aryl moieties of R⁹ are unsubstituted or substituted with groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R⁹ is selected from the group consisting of H, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl, and OR^{Y}.

According to one embodiment of formula I, R⁹ is H.

According to still another embodiment of formula I, R⁹ is halogen, in particular F, Cl, Br or I, more specifically F, Cl or Br, in particular F or Cl.

According to still another embodiment of formula I, R⁹ is F.

According to still another embodiment of formula I, R⁹ is Cl.

According to still another embodiment of formula I, R⁹ is Br.

According to still another embodiment of formula I, R⁹ is OH.

According to still another embodiment of formula I, R⁹ is CN.

According to still another embodiment of formula I, R⁹ is NO₂.

According to still another embodiment of formula I, R⁹ is SH.

According to still another embodiment of formula I, R⁹ is NH₂.

According to still another embodiment of formula I, R⁹ is, NH(C₁-C₄-alkyl), in particular NH(CH₃), NH(C₂H₅).

According to still another embodiment of formula I, R⁹ is N(C₁-C₄-alkyl)₂, in particular NH(CH₃)₂, NH(C₂H₅)₂.

According to still another embodiment of formula I, R⁹ is, NH(C₂-C₄-alkenyl), in particular NH(CH═CH₂), NH(CH₂CH═CH₂).

According to still another embodiment of formula I, R⁹ is, N(C₂-C₄-alkenyl)₂, in particular N(CH═CH₂)₂, N(CH₂CH═CH₂)₂.

According to still another embodiment of formula I, R⁹ is, NH(C₂-C₄-alkynyl), in particular NH(C≡CH), NH(CH₂C≡CH).

According to still another embodiment of formula I, R⁹ is, N(C₂-C₄-alkynyl)₂, in particular N(C≡CH)₂, N(CH₂C≡CH)₂.

According to still another embodiment of formula I, R⁹ is, NH(C₃-C₆-cycloalkyl), in particular NH(C₃H₇), NH(C₄H₉).

According to still another embodiment of formula I, R⁹ is, N(C₃-C₆-cycloalkyl)₂, in particular N(C₃H₇)₂, N(C₄H₉)₂.

According to still another embodiment of formula I, R⁹ is N(C₁-C₄-alkyl)(C₂-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

According to still another embodiment of formula I, R⁹ is N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

According to still another embodiment of formula I, R⁹ is N(C₁-C₄-alkyl)(C₃-C₆-cycloalkyl), in particular N(CH₃)(C₃H₇), N(CH₃)(C₄H₉), N(C₂H₅)(C₃H₇), N(CH₃)(C₄H₉).

According to still another embodiment of formula I, R⁹ is N(C₂-C₄-alkenyl)(C₂-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

According to still another embodiment of formula I, R⁹ is N(C₂-C₄-alkenyl)(C₃-C₆-cycloalkyl), in particular N(CH═CH₂)(C₃H₇), N(CH₂CH═CH₂)(C₄H₉), N(CH═CH₂)(C₃H₇), N(CH₂CH═CH₂)(C₄H₉).

According to still another embodiment of formula I, R⁹ is N(C₂-C₄-alkynyl)(C₃-C₆-cycloalkyl), in particular N(C≡CH)(C₃H₇), N(CH₂C≡CH)(C₄H₉), N(C≡CH)(C₃H₇), N(CH₂C≡CH)(C₄H₉).

According to still another embodiment of formula I, R⁹ is, NH(C(═O)(C₁-C₄-alkyl), in particular NH(C(═O)(CH₃), NH(C(═O)(C₂H₅).

According to still another embodiment of formula I, R⁹ is N(C(═O)(C₁-C₄-alkyl)₂, in particular N(C(═O)(CH₃)₂, N(C(═O)(C₂H₅)₂.

According to a further specific embodiment of formula I, R⁹ is NH—SO₂—R^{x} such as NH—SO₂—CH₃, NH—SO₂—CH₂—CH₃, NH—SO₂—CF₃, NH—SO₂-Ts.

According to still another embodiment of formula I, R⁹ is S(O)ₙ—C₁-C₆-alkyl such as SCH₃, S(═O) CH₃, S(O)₂CH₃.

According to still another embodiment of formula I, R⁹ is S(O)ₙ-aryl such as S-phenyl, S(═O) phenyl, S(O)₂phenyl.

According to still another embodiment of formula I, R⁹ is S(O)ₙ—C₂-C₆-alkenyl such as SCH═CH₂, S(═O)CH═CH₂, S(O)₂CH═CH₂, SCH₂CH═CH₂, S(═O)CH₂CH═CH₂, S(O)₂CH₂CH═CH₂.

According to still another embodiment of formula I, R⁹ is S(O)ₙ—C₂-C₆-alkynyl such as SC≡CH, S(═O)C≡CH, S(O)₂C≡CH, SCH₂C≡CH, S(═O)CH₂C≡CH, S(O)₂CH₂C≡CH.

According to a further specific embodiment of formula I, R⁹ is CH(═O).

According to a further specific embodiment of formula I, R⁹ is C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁹ is C(═O)C₂-C₆-alkenyl, C(═O)O(C₂-C₆-alkenyl), (═O)NH(C₂-C₆-alkenyl) or C(═O)N(C₂-C₆-alkenyl)₂, wherein alkenyl is CH═CH₂, CH₂CH═CH₂.

According to a further specific embodiment of formula I, R⁹ is C(═O)C₂-C₆-alkynyl, C(═O)O(C₂-C₆-alkynyl), C(═O)NH(C₂-C₆-alkynyl) or C(═O)N(C₂-C₆-alkynyl)₂, wherein alkynyl is C≡CH, CH₂C≡CH.

According to a further specific embodiment of formula I, R⁹ is C(═O)C₃-C₆-cycloalkyl, C(═O)O(C₃-C₆-cycloalkyl), C(═O)NH(C₃-C₆-cycloalkyl) or C(═O)N(C₃-C₇-cycloalkyl)₂, wherein cycloalkyl is cyclopropyl (C₃H₇) or cyclobutyl (C₄H₉).

According to a further specific embodiment of formula I, R⁹ is CH(═S).

According to a further specific embodiment of formula I, R⁹ is C(═S)C₁-C₆-alkyl, C(═S)OC₁-C₆-alkyl, C(═S)NH(C₁-C₆-alkyl) or C(═S)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R⁹ is C(═S)C₂-C₆-alkenyl, C(═S)OC₂-C₆-alkenyl, C(═S)NH(C₂-C₆-alkenyl) or C(═S)N(C₂-C₆-alkenyl)₂, wherein alkenyl is CH═CH₂, CH₂CH═CH₂.

According to a further specific embodiment of formula I, R⁹ is C(═S)C₂-C₆-alkynyl, C(═S)O(C₂-C₆-alkynyl), C(═S)NH(C₂-C₆-alkynyl) or C(═S)N(C₂-C₆-alkynyl)₂, wherein alkynyl is C≡CH, CH₂C≡CH.

According to a further specific embodiment of formula I, R⁹ is C(═S)C₃-C₆-cycloalkyl, C(═S)O(C₃-C₇-cycloalkyl), C(═S)NH(C₃-C₇-cycloalkyl) or, C(═S)N(C₃-C₇-cycloalkyl)₂, wherein cycloalkyl is cyclopropyl (C₃H₇) or cyclobutyl (C₄H₉).

According to still another embodiment of formula I, R⁹ is C₁-C₆-alkyl, in particular C₁-C₄-alkyl, such as CH₃. or C₂H₅, in particular CH₃ or CH₂CH₃.

According to still another embodiment of formula I, R⁹ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still a further embodiment of formula I, R⁹ is C₂-C₆-alkenyl, in particular C₂-C₄-alkenyl, such as CH═CH₂, C(CH₃)═CH₂, CH₂CH═CH₂.

According to a further specific embodiment of formula I, R⁹ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R⁹ is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R⁹ is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl.

According to a further specific embodiment of formula I, R⁹ is OR, wherein R^{Y} is C₁-C₆-alkyl, in particular C₁-C₄-alkyl, more specifically C₁-C₂-alkoxy. R⁹ is such as OCH₃ or OCH₂CH₃.

According to a further specific embodiment of formula I, R⁹ is OR^{Y}, wherein R^{Y} is C₁-C₆-halogenalkyl, in particular C₁-C₄-halogenalkyl, more specifically C₁-C₂-halogenalkyl. R⁹ is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R⁹ is OR, wherein R^{Y} C₂-C₆-alkenyl, in particular C₂-C₄-alkenyl, more specifically C₁-C₂-alkenyl. R⁹ is such as OCH═CH₂, OCH₂CH═CH₂.

According to a further specific embodiment of formula I, R⁹ is OR^{Y}, wherein R^{Y} C₂-C₆-halogenalkenyl, in particular C₂-C₄-halogenalkenyl, more specifically C₁-C₂-halogenalkenyl.

According to a further specific embodiment of formula I, R⁹ is OR^{Y}, wherein R^{Y} C₂-C₆-alkynyl, in particular C₂-C₆-alkynyl, in particular C₂-C₄-alkynyl, more specifically C₁-C₂-alkynyl. R⁹ is such as OC≡CH,

According to a further specific embodiment of formula I, R⁹ is OR^{Y}, wherein R^{Y} C₂-C₆-halogenalkynyl, in particular C₂-C₆-halogenalkynyl, in particular C₂-C₄-halogenalkynyl, more specifically C₁-C₂-halogenalkynyl. R⁹ is such as OC≡CCl, OCH₂C≡CCl, or OCCl₂C≡CCl.

According to still another embodiment of formula I, R⁹ is is OR^{Y}, wherein R^{Y} C₃-C₆-cycloalkenyl, in particular cyclopropenyl.

According to still another embodiment of formula I, R⁹ is C₃-C₆-cycloalkyl, in particular cyclopropyl.

According to still another embodiment of formula I, R⁹ is C₃-C₆-halogencycloalkyl. In a special embodiment R^{9b} is fully or partially halogenated cyclopropyl, such as 1-F-cyclopropyl, 1-Cl-cyclopropyl, 1,1-F₂-cyclopropyl, 1,1-Cl₂-cyclopropyl.

According to still another embodiment of formula I, R⁹ is phenyl-C₁-C₆-alkyl, such as phenyl-CH₂, wherein the phenyl moiety in each case is unsubstituted or substituted by one, two or three identical or different groups R^{9b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R⁹ is aryl, in particular phenyl, wherein the aryl or phenyl moiety in each case is unsubstituted or substituted with identical or different groups R^{9b} which independently of one another are selected from CN, halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, OCHF₂, CF₃ and OCF₃. According to one embodiment, R⁹ is unsubstituted phenyl. According to another embodiment, R⁹ is phenyl, that is substituted with one, two or three, in particular one, halogen, in particular selected from F, Cl and Br, more specifically selected from F and Cl.

According to still another embodiment of formula I, R⁹ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁹ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R⁹ is in each case independently selected from H, halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl and C₃-C₆-halogencycloalkyl, wherein the acyclic moieties of R⁹ are unsubstituted or substituted with identical or different groups R^{9a} as defined and preferably defined herein, and wherein the carbocyclic, phenyl and heteroaryl moieties of R⁹ are unsubstituted or substituted with identical or different groups R^{9b} as defined and preferably defined herein.

According to still another embodiment of formula I, R⁹ is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy; wherein the acyclic moieties of R⁹ are unsubstituted or substituted with identical or different groups R^{9a} as defined and preferably defined herein.

According to still another embodiment of formula I, R⁹ is in each case independently selected from CN, halogen, C₁-C₆-alkyl, C₁-C₆-halogenalkyl, C₂-C₆-alkynyl, OR^{Y}, C₃-C₆-cycloalkyl; wherein R^{Y} is C₁-C₆-alkyl, C₁-C₆-halogenalkyl, C₂-C₆-alkenyl or C₂-C₆-alkynyl.

R^{9a} are the possible substituents for the acyclic moieties of R⁹.

According to one embodiment R^{9a} is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{91a} selected from the group consisting of halogen, OH, C₁-C₄-alkyl, C₁-C₄-halogenalkyl, C₁-C₄-alkoxy and C₁-C₄-halogenalkoxy.

According to one embodiment R^{9a} is independently selected from halogen, C₁-C₆-alkoxy, C₃-C₆-cycloalkyl, C₃-C₆-halogencycloalkyl and C₁-C₄-halogenalkoxy. Specifically, R^{9a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R^{9a} is independently halogen, in particular selected from F, Cl, Br and I, more specifically F, Cl and Br.

R^{9b} are the possible substituents for the carbocyclic, heteroaryl and phenyl moieties of R⁹. R^{9b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{9b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{9b} is independently selected from F, Cl, CN, CH₃, OCH₃ and halogenmethoxy.

Particularly preferred embodiments of R⁹ according to the invention are in Table P9 below, wherein each line of lines P9-1 to P9-43 corresponds to one particular embodiment of the invention, wherein P9-1 to P9-43 are also in any combination with one another a preferred embodiment of the present invention. The connection point to the carbon atom, to which R⁹ is bound is marked with “#” in the drawings.

TABLE P9

| No. | R⁹ |
|---|---|
| P9-1 | H |
| P9-2 | CH₃ |
| P9-3 | CH₂F |
| P9-4 | CHF₂ |
| P9-5 | CF₃ |
| P9-6 | C₂H₅ |
| P9-7 | CH(CH₃)₂ |
| P9-8 | CH₂CH₂CH₃ |
| P9-9 | CH₂CH₂CH₂CH₃ |
| P9-10 | CH₂CH(CH₃)₂ |
| P9-11 | C(CH₃)₃ |
| P9-12 | CH₂CH₂CH₂CH₂CH₃ |
| P9-13 | CH═CH₂ |
| P9-14 | CH₂CH═CH₂ |
| P9-15 | C≡CH |
| P9-16 | CH₂C≡CH |
| P9-17 | CH₂CH₂CH(CH₃)₂ |
| P9-18 | OH |
| P9-19 | OCH₃ |
| P9-20 | OCHF₂ |
| P9-21 | OC₂H₅ |
| P9-22 | CN |
| P9-23 | F |
| P9-24 | Cl |
| P9-25 | Br |
| P9-26 | NO₂ |
| P9-27 | NH₂ |
| P9-28 | CO—NH₂ |
| P9-29 | CO—NH(CH₃) |
| P9-30 | CO—N(CH₃)₂ |
| P9-31 | HNCH₃ |
| P9-32 | HNC₂H₅ |
| P9-33 | (CH₃)₂N |
| P9-34 | SO₂H |
| P9-35 | SO₂—CH₃ |
| P9-36 | SO—CH₃ |
| P9-37 | S—CH₃ |
| P9-38 | 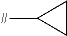 |
| P9-39 | 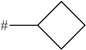 |
| P9-40 | 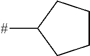 |
| P9-41 | 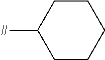 |
| P9-42 | 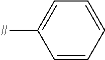 |
| P9-43 | 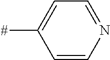 |

R¹⁰ is in each case independently selected from H, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₂-C₄-alkyl)(C₂-C₄-alkenyl), N(C₂-C₄-alkyl)(C₂-C₄-alkynyl), N(C₂-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is as defined above;
wherein the acyclic moieties of R¹⁰ are unsubstituted or substituted with groups R^{10a} which independently of one another are selected from:
R^{10a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{10a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R¹⁰ are unsubstituted or substituted with groups R^{10b} which independently of one another are selected from:
R^{10b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R¹⁰ is selected from the group consisting of H, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy and OR^{Y}.

According to one embodiment of formula I, R¹⁰ is H.

R¹⁰ is selected from the group consisting of halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy and OR^{Y}.

According to still another embodiment of formula I, R¹⁰ is halogen, in particular F, Cl, Br or I, more specifically F, Cl or Br, in particular F or Cl.

According to still another embodiment of formula I, R¹⁰ is F.

According to still another embodiment of formula I, R¹⁰ is Cl.

According to still another embodiment of formula I, R¹⁰ is Br.

According to still another embodiment of formula I, R¹⁰ is OH.

According to still another embodiment of formula I, R¹⁰ is CN.

According to still another embodiment of formula I, R¹⁰ is NO₂.

According to still another embodiment of formula I, R¹⁰ is SH.

According to still another embodiment of formula I, R¹⁰ is NH₂.

According to still another embodiment of formula I, R¹⁰ is, NH(C₁-C₄-alkyl), in particular NH(CH₃), NH(C₂H₅).

According to still another embodiment of formula I, R¹⁰ is, N(C₁-C₄-alkyl)₂, in particular NH(CH₃)₂, NH(C₂H₅)₂.

According to still another embodiment of formula I, R¹⁰ is, NH(C₂-C₄-alkenyl), in particular NH(CH═CH₂), NH(CH₂CH═CH₂).

According to still another embodiment of formula I, R¹⁰ is, N(C₂-C₄-alkenyl)₂, in particular N(CH═CH₂)₂, N(CH₂CH═CH₂)₂.

According to still another embodiment of formula I, R¹⁰ is, NH(C₂-C₄-alkynyl), in particular NH(C≡CH), NH(CH₂C≡CH).

According to still another embodiment of formula I, R¹⁰ is, N(C₂-C₄-alkynyl)₂, in particular N(C≡CH)₂, N(CH₂C≡CH)₂.

According to still another embodiment of formula I, R¹⁰ is, NH(C₃-C₆-cycloalkyl), in particular NH(C₃H₇), NH(C₄H₉).

According to still another embodiment of formula I, R¹⁰ is, N(C₃-C₆-cycloalkyl)₂, in particular N(C₃H₇)₂, N(C₄H₉)₂.

According to still another embodiment of formula I, R¹⁰ is N(C₁-C₄-alkyl)(C₂-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

According to still another embodiment of formula I, R¹⁰ is N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

According to still another embodiment of formula I, R¹⁰ is N(C₁-C₄-alkyl)(C₃-C₆-cycloalkyl), in particular N(CH₃)(C₃H₇), N(CH₃)(C₄H₉), N(C₂H₅)(C₃H₇), N(CH₃)(C₄H₉).

According to still another embodiment of formula I, R¹⁰ is N(C₂-C₄-alkenyl)(C₂-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

According to still another embodiment of formula I, R¹⁰ is N(C₂-C₄-alkenyl)(C₃-C₆-cycloalkyl), in particular N(CH═CH₂)(C₃H₇), N(CH₂CH═CH₂)(C₄H₉), N(CH═CH₂)(C₃H₇), N(CH₂CH═CH₂)(C₄H₉).

According to still another embodiment of formula I, R¹⁰ is N(C₂-C₄-alkynyl)(C₃-C₆-cycloalkyl), in particular N(C≡CH)(C₃H₇), N(CH₂C≡CH)(C₄H₉), N(C≡CH)(C₃H₇), N(CH₂C≡CH)(C₄H₉).

According to still another embodiment of formula I, R¹⁰ is, NH(C(═O)(C₁-C₄-alkyl), in particular NH(C(═O)(CH₃), NH(C(═O)(C₂H₅).

According to still another embodiment of formula I, R¹⁰ is N(C(═O)(C₁-C₄-alkyl)₂, in particular N(C(═O)(CH₃)₂, N(C(═O)(C₂H₅)₂.

According to a further specific embodiment of formula I, R¹⁰ is NH—SO₂—R^{x} such as NH—SO₂—CH₃, NH—SO₂—CH₂—CH₃, NH—SO₂—CF₃, NH—SO₂-Ts.

According to still another embodiment of formula I, R¹⁰ is S(O)ₙ—C₁-C₆-alkyl such as SCH₃, S(═O) CH₃, S(O)₂CH₃.

According to still another embodiment of formula I, R¹⁰ is S(O)ₙ-aryl such as S-phenyl, S(═O) phenyl, S(O)₂phenyl.

According to still another embodiment of formula I, R¹⁰ is S(O)ₙ—C₂-C₆-alkenyl such as SCH═CH₂, S(═O)CH═CH₂, S(O)₂CH═CH₂, SCH₂CH═CH₂, S(═O)CH₂CH═CH₂, S(O)₂CH₂CH═CH₂.

According to still another embodiment of formula I, R¹⁰ is S(O)ₙ—C₂-C₆-alkynyl such as SC≡CH, S(═O)C≡CH, S(O)₂C≡CH, SCH₂C≡CH, S(═O)CH₂C≡CH, S(O)₂CH₂C≡CH.

According to a further specific embodiment of formula I, R¹⁰ is CH(═O).

According to a further specific embodiment of formula I, R¹⁰ is C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R¹⁰ is C(═O)C₂-C₆-alkenyl, C(═O)O(C₂-C₆-alkenyl), C(═O)NH(C₂-C₆-alkenyl) or C(═O)N(C₂-C₆-alkenyl)₂, wherein alkenyl is CH═CH₂, C(CH₃)═CH₂, CH₂CH═CH₂.

According to a further specific embodiment of formula I, R¹⁰ is C(═O)C₂-C₆-alkynyl, C(═O)O(C₂-C₆-alkynyl), C(═O)NH(C₂-C₆-alkynyl) or C(═O)N(C₂-C₆-alkynyl)₂), wherein alkynyl is C≡CH, CH₂C≡CH,

According to a further specific embodiment of formula I, R¹⁰ is C(═O)C₃-C₆-cycloalkyl, C(═O)O(C₃-C₆-cycloalkyl), C(═O)NH(C₃-C₆-cycloalkyl) or C(═O)N(C₃-C₇-cycloalkyl)₂, wherein cycloalkyl is cyclopropyl (C₃H₇) or cyclobutyl (C₄H₉).

According to a further specific embodiment of formula I, R¹⁰ is CH(═S).

According to a further specific embodiment of formula I, R¹⁰ is C(═S)C₁-C₆-alkyl, C(═S)OC₁-C₆-alkyl, C(═S)NH(C₁-C₆-alkyl) or C(═S)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R¹⁰ is C(═S)C₂-C₆-alkenyl, C(═S)OC₂-C₆-alkenyl, C(═S)NH(C₂-C₆-alkenyl) or C(═S)N(C₂-C₆-alkenyl)₂, wherein alkenyl is CH═CH₂, CH₂CH═CH₂.

According to a further specific embodiment of formula I, R¹⁰ is C(═S)C₂-C₆-alkynyl, C(═S)O(C₂-C₆-alkynyl), C(═S)NH(C₂-C₆-alkynyl) or C(═S)N(C₂-C₆-alkynyl), wherein alkynyl is C≡CH, CH₂C≡CH.

According to a further specific embodiment of formula I, R¹⁰ is C(═S)C₃-C₆-cycloalkyl, C(═S)O(C₃-C₇-cycloalkyl), C(═S)NH(C₃-C₇-cycloalkyl) or, C(═S)N(C₃-C₇-cycloalkyl)₂, wherein cycloalkyl is cyclopropyl (C₃H₇) or cyclobutyl (C₄H₉).

According to still another embodiment of formula I, R¹⁰ is C₁-C₆-alkyl, in particular C₁-C₄-alkyl, such as CH₃. or C₂H₅, in particular CH₃ or CH₂CH₃.

According to still another embodiment of formula I, R¹⁰ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still a further embodiment of formula I, R¹⁰ is C₂-C₆-alkenyl, in particular C₂-C₄-alkenyl, such as CH═CH₂.

According to a further specific embodiment of formula I, R¹⁰ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R¹⁰ is C₂-C₆-alkynyl or C₂-C₆-halogenalkynyl, in particular C₂-C₄-alkynyl or C₂-C₄-halogenalkynyl, such as C≡CH, CH₂ C≡CH, C≡CCl, CH₂C≡CCl, or CCl₂C≡CCl.

According to a further specific embodiment of formula I, R¹⁰ is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl.

According to a further specific embodiment of formula I, R¹⁰ is OR^{Y}, wherein R^{Y} is C₁-C₆-alkyl, in particular C₁-C₄-alkyl, more specifically C₁-C₂-alkoxy. R¹⁰ is such as OCH₃ or OCH₂CH₃.

According to a further specific embodiment of formula I, R¹⁰ is OR^{Y}, wherein R^{Y} is C₁-C₆-halogenalkyl, in particular C₁-C₄-halogenalkyl, more specifically C₁-C₂-halogenalkyl. R¹⁰ is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R¹⁰ is OR^{Y}, wherein R^{Y} C₂-C₆-alkenyl, in particular C₂-C₄-alkenyl, more specifically C₁-C₂-alkenyl. R¹⁰ is such as OCH═CH₂, OCH₂CH═CH₂.

According to a further specific embodiment of formula I, R¹⁰ is OR^{Y}, wherein R^{Y} C₂-C₆-alkynyl, in particular C₂-C₆-alkynyl, in particular C₂-C₄-alkynyl, more specifically C₁-C₂-alkynyl. R¹⁰ is such as OC≡CH, OC≡CCl, OCH₂C≡CCl, or OCCl₂C≡CCl

According to still another embodiment of formula I R¹⁰ is OR^{Y}, wherein R^{Y} is C₃-C₆-cycloalkyl, in particular cyclopropyl.

According to still another embodiment of formula I, R¹⁰ is OR^{Y}, wherein R^{Y} is C₃-C₆-halogencycloalkyl.

In a special embodiment R¹ is fully or partially halogenated cyclopropyl.

According to still another embodiment of formula I, R¹⁰ is is OR^{Y}, wherein R^{Y} C₃-C₆-cycloalkenyl, in particular cyclopropenyl.

According to still another embodiment of formula I, R¹⁰ is C₃-C₆-cycloalkyl, in particular cyclopropyl.

According to still another embodiment of formula I, R¹⁰ is C₃-C₆-halogencycloalkyl. In a special embodiment R^{10b} is fully or partially halogenated cyclopropyl, such as 1-F-cyclopropyl, 1-Cl-cyclopropyl, 1,1-F₂-cyclopropyl, 1,1-Cl₂-cyclopropyl

According to still another embodiment of formula I, R¹⁰ is phenyl-C₁-C₆-alkyl, such as phenyl-CH₂, wherein the phenyl moiety in each case is unsubstituted or substituted by one, two or three identical or different groups R^{10b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R¹⁰ is aryl, in particular phenyl, wherein the aryl or phenyl moiety in each case is unsubstituted or substituted with identical or different groups R^{10b} which independently of one another are selected from CN, halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, OCHF₂, CF₃ and OCF₃. According to one embodiment, R¹⁰ is unsubstituted phenyl. According to another embodiment, R¹⁰ is phenyl, that is substituted with one, two or three, in particular one, halogen, in particular selected from F, Cl and Br, more specifically selected from F and Cl.

According to still another embodiment of formula I, R¹⁰ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁹ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R¹⁰ is in each case independently selected from H, halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl and C₃-C₆-halogencycloalkyl, wherein the acyclic moieties of R¹⁰ are unsubstituted or substituted with identical or different groups R^{10a} as defined and preferably defined herein, and wherein the carbocyclic, phenyl and heteroaryl moieties of R¹⁰ are unsubstituted or substituted with identical or different groups R^{10b} as defined and preferably defined herein.

According to still another embodiment of formula I, R¹⁰ is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy; wherein the acyclic moieties of R¹⁰ are unsubstituted or substituted with identical or different groups R^{10a} as defined and preferably defined herein.

According to still another embodiment of formula I, R¹⁰ is in each case independently selected from CN, halogen, C₁-C₆-alkyl, C₁-C₆-halogenalkyl, C₂-C₆-alkynyl, OR^{Y}, C₃-C₆-cycloalkyl; wherein R^{Y} is C₁-C₆-alkyl, C₁-C₆-halogenalkyl, C₂-C₆-alkenyl or C₂-C₆-alkynyl.

R^{10a} are the possible substituents for the acyclic moieties of R¹⁰.

According to one embodiment R^{10a} is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{101a} selected from the group consisting of halogen, OH, C₁-C₄-alkyl, C₁-C₄-halogenalkyl, C₁-C₄-alkoxy and C₁-C₄-halogenalkoxy.

According to one embodiment R^{10a} is independently selected from halogen, C₁-C₆-alkoxy, C₃-C₆-cycloalkyl, C₃-C₆-halogencycloalkyl and C₁-C₄-halogenalkoxy. Specifically, R^{10a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R¹⁰⁰ is independently halogen, in particular selected from F, Cl, Br and I, more specifically F, Cl and Br.

R^{10b} are the possible substituents for the carbocyclic, heteroaryl and phenyl moieties of R¹⁰. R^{10b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{10b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{10b} is independently selected from F, Cl, CN, CH₃, OCH₃ and halogenmethoxy.

Particularly preferred embodiments of R¹⁰ according to the invention are in Table P10 below, wherein each line of lines P10-1 to P10-43 corresponds to one particular embodiment of the invention, wherein P10-1 to P10-43 are also in any combination with one another a preferred embodiment of the present invention. The connection point to the carbon atom, to which R¹⁰ is bound is marked with “#” in the drawings.

TABLE P10

| No. | R¹⁰ |
|---|---|
| P10-1 | H |
| P10-2 | CH₃ |
| P10-3 | CH₂F |
| P10-4 | CHF₂ |
| P10-5 | CF₃ |
| P10-6 | C₂H₅ |
| P10-7 | CH(CH₃)₂ |
| P10-8 | CH₂CH₂CH₃ |
| P10-9 | CH₂CH₂CH₂CH₃ |
| P10-10 | CH₂CH(CH₃)₂ |
| P10-11 | C(CH₃)₃ |
| P10-12 | CH₂CH₂CH₂CH₂CH₃ |
| P10-13 | CH═CH₂ |
| P10-14 | CH₂CH═CH₂ |
| P10-15 | C≡CH |
| P10-16 | CH₂C≡CH |
| P10-17 | CH₂CH₂CH(CH₃)₂ |
| P10-18 | OH |
| P10-19 | OCH₃ |
| P10-20 | OCHF₂ |
| P10-21 | OC₂H₅ |
| P10-22 | CN |
| P10-23 | F |
| P10-24 | Cl |
| P10-25 | Br |
| P10-26 | NO₂ |
| P10-27 | NH₂ |
| P10-28 | CO—NH₂ |
| P10-29 | CO—NH(CH₃) |
| P10-30 | CO—N(CH₃)₂ |
| P10-31 | HNCH₃ |
| P10-32 | HNC₂H₅ |
| P10-33 | (CH₃)₂N |
| P10-34 | SO₂H |
| P10-35 | SO₂—CH₃ |
| P10-36 | SO—CH₃ |
| P10-37 | S—CH₃ |
| P10-38 | 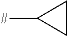 |
| P109-39 | 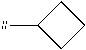 |
| P10-40 | 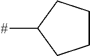 |
| P10-41 | 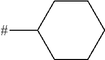 |
| P10-42 | 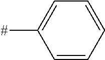 |
| P10-43 | 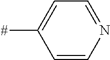 |

According to still another embodiment of formula I, R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent RN selected from C₁-C₄-alkyl, C₁-C₄-halogenalkyl and SO₂Ph, wherein Ph is unsubstituted or substituted with substituents selected from C₁-C₄-alkyl, halogen, C₁-C₄-halogenalkyl, C₁-C₄-alkoxy and C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ wherein m is 0, 1, 2, 3 or 4;
R^{N} is the substituent of the heteroatom N that is contained in the heterocycle formed by R⁹ and R¹⁰ in some of the inventive compounds. R^{N} is selected from C₁-C₄-alkyl, C₁-C₄-halogenalkyl and SO₂Ph, wherein Ph is unsubstituted phenyl or phenyl that is substituted with one, two or three substituents selected from C₁-C₄-alkyl. In one preferred embodiment, R^{N} is in each case independently selected from C₁-C₂-alkyl, C₁-C₂-halogenalkyl and SO₂Ph, wherein Ph is unsubstituted phenyl or phenyl that is substituted with one methyl substituents. In one particular embodiment, R^{N} is in each case independently selected from C₁-C₂-alkyl, more particularly methyl.

In one particular embodiment, R^{N} is in each case independently selected from SO₂Ph, wherein Ph is unsubstituted phenyl or phenyl that is substituted with one methyl.

According to still another embodiment of formula I, R⁹ and R¹⁰ together with the carbon atoms to which they are bound form a saturated or partially unsaturated five-, six- or seven-membered carbo- and heterocycle that is unsubstituted or substituted.

According to one embodiment, R⁹ and R¹⁰ form a 3-membered saturated carbocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 4-membered saturated carbocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 5-membered saturated carbocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 6-membered saturated carbocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 7-membered saturated carbocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 3-membered saturated heterocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 4-membered saturated heterocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 5-membered saturated heterocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 6-membered saturated heterocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 7-membered saturated heterocycle. According to one embodiment thereof, the carbocycle is unsubstituted, i.e. it does not carry any substituent R¹¹. According to still another embodiment of formula I, it is substituted with R¹¹.

According to one embodiment, R⁹ and R¹⁰ form a 5-membered saturated heteroaryl. According to one embodiment thereof, the heteroaryl is unsubstituted, i.e. it does not carry any substituent R¹¹. According to still another embodiment of formula I, it is substituted with R¹¹.

According to one embodiment, R⁹ and R¹⁰ form a 6-membered heteroaryl. According to one embodiment thereof, the heteroaryl is unsubstituted, i.e. it does not carry any substituent R¹¹. According to still another embodiment of formula I, it is substituted with R¹¹.

R¹¹ according to the invention is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, saturated or partially unsaturated three-, four-, five-, six-, seven-, eight-, nine-, or ten-membered carbo- and heterocycle, five- or six-membered heteroaryl and aryl; wherein the heterocycle and heteroaryl contains one, two or three heteroatoms selected from N, O and S; and wherein in each case one or two CH₂ groups of the carbo- or heterocycle may be replaced by a group independently selected from C(═O) and C(═S); and wherein

R^{x} is as defined above;
wherein the acyclic moieties of R¹¹ are unsubstituted or substituted with identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or unsubstituted or substituted with R^{11a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

For every R¹¹ that is present in the inventive compounds, the following embodiments and preferences apply independently of the meaning of any other R¹¹ that may be present in the ring.

According to one embodiment of formula I, wherein m is 0, 1, 2, 3 or 4.

According to still another embodiment of formula I, m is 0.

According to still another embodiment of formula I, m is 1.

According to still another embodiment of formula I, m is 2 or 3. According to one specific embodiment thereof, m is 2. According to still another embodiment of formula I, m is 3.

According to one embodiment of formula I, R¹¹ is halogen, C₁-C₆-alkyl, C₁-C₆-halogenalkyl, C₁-C₆-alkoxy or, C₁-C₆-halogenalkoxy, in particular CH₃, Et, CHF₂, OCH₃, OCHF₂, OCF₃, F, Cl, more specifically H, CH₃, F or Cl most preferred F or Cl.

According to still another embodiment of formula I, R¹¹ is halogen, in particular Br, F or Cl, more specifically F or Cl.

According to still another embodiment of formula I, R¹¹ is OH.

According to still another embodiment of formula I, R¹¹ is CN.

According to still another embodiment of formula I R¹¹ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x1} independently selected from C₁-C₄-alkyl.

According to still another embodiment of formula I, R¹¹ is C₁-C₆-alkyl, in particular C₁-C₄-alkyl, such as CH₃.

According to still another embodiment of formula I, R¹¹ is C₁-C₆-halogenalkyl, in particular C₁-C₄-halogenalkyl, such as CF₃, CHF₂, CH₂F, CCl₃, CHCl₂ or CH₂Cl.

According to still another embodiment of formula I, R¹¹ is C₂-C₆-alkenyl or C₂-C₆-halogenalkenyl, in particular C₂-C₄-alkenyl or C₂-C₄-halogenalkenyl, such as CH═CH₂, C(CH₃)═CH₂, CH₂CH═CH₂, CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still another embodiment of formula I, R¹¹ is C₂-C₆-alkynyl or C₂-C₆-halogenalkynyl, in particular C₂-C₄-alkynyl or C₂-C₄-halogenalkynyl, such as C≡CH, CH₂C≡CH, C≡C—Cl, C≡C—CH₃, CH₂C≡CH, CH₂C≡CCl or CH₂C≡C—CH₃.

According to still another embodiment of formula I, R¹¹ is C₁-C₆-alkoxy, in particular C₁-C₄-alkoxy, more specifically C₁-C₂-alkoxy such as OCH₃ or OCH₂CH₃.

According to still another embodiment of formula I, R¹¹ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R¹¹ is C₃-C₆-cycloalkyl, in particular cyclopropyl.

According to still another embodiment of formula I, R¹¹ is C₃-C₆-cycloalkyl, for example cyclopropyl, substituted with one, two, three or up to the maximum possible number of identical or different groups R^{11b} as defined and preferably herein.

According to still another embodiment of formula I, R¹¹ is C₃-C₆-halogencycloalkyl. In a special embodiment R¹¹ is fully or partially halogenated cyclopropyl.

According to still another embodiment of formula I, R¹¹ is unsubstituted aryl or aryl that is substituted with one, two, three or four R^{11b}, as defined herein. In particular, R¹¹ is unsubstituted phenyl or phenyl that is substituted with one, two, three or four R^{11b}, as defined herein.

According to still another embodiment of formula I, R¹¹ is unsubstituted 5- or 6-membered heteroaryl. According to still a further embodiment, R¹¹ is 5- or 6-membered heteroaryl that is substituted with one, two or three R^{11b}, as defined herein.

According to still another embodiment of formula I, R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R¹¹ are not further substituted or carry one, two, three, four or five identical or different groups R^{11a} as defined below and wherein the carbocyclic, heterocyclic and heteroaryl moieties of R¹¹ are not further substituted or carry one, two, three, four or five identical or different groups R^{11b} as defined below.

According to still another embodiment of formula I, R¹¹ is independently selected from halogen, OH, C₁-C₆-alkyl, C₁-C₆-halogenalkyl, C₁-C₆-alkoxy and C₁-C₆-halogenalkoxy, in particular independently selected from F, Cl, Br, CN, OH, C₁-C₄-alkyl, C₁-C₄-halogenalkyl, C₁-C₄-alkoxy and C₁-C₄-halogenalkoxy.

R^{11a} are the possible substituents for the acyclic moieties of R¹¹.

R^{11a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio.

R^{11a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or unsubstituted or substituted with R^{111a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy, C₁-C₂-halogenalkoxy, more specifically selected from halogen, such as F, Cl and Br.

In to one embodiment R^{11a} is independently selected from halogen, OH, CN, C₁-C₂-alkoxy, C₃-C₆-cycloalkyl, C₃-C₆-halogencycloalkyl and C₁-C₂-halogenalkoxy. Specifically, R^{11a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{11a} is independently selected from halogen, such as F, Cl, Br and I, more specifically F, Cl and Br.

According to still another embodiment of formula I, R^{11a} is independently selected from OH, C₃-C₆-cycloalkyl, C₃-C₆-halogencycloalkyl and C₁-C₂-halogenalkoxy. Specifically, R^{11a} is independently selected from OH, cyclopropyl and C₁-C₂-halogenalkoxy.

R^{11b} are the possible substituents for the carbocyclic, heterocyclic and heteroaryl moieties of R¹¹.

R^{11b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl and C₁-C₄-halogenalkoxy.

According to one embodiment thereof R^{11b} is independently selected from halogen, CN, C₁-C₂-alkyl, C₁-C₂-alkoxy, C₁-C₂-halogenalkyl, C₃-C₆-cycloalkyl, C₃-C₆-halogencycloalky and C₁-C₂-halogenalkoxy. Specifically, R^{11b} is independently selected from F, Cl, OH, CN, CH₃, OCH₃, cyclopropyl, 1-F-cyclopropyl, 1-Cl-cyclopropyl, 1,1-F₂-cyclopropyl, 1,1-Cl₂-cyclopropyl and halogenmethoxy.

According to still another embodiment thereof R^{11b} is independently selected from C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{1b} is independently selected from OH, CH₃, OCH₃, cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from OH, CH₃, OCH₃, cyclopropyl, 1-F-cyclopropyl, 1-Cl-cyclopropyl, 1,1-F₂-cyclopropyl, 1,1-Cl₂-cyclopropyl cyclopropyl and OCHF₂.

Particularly preferred embodiments of combinations of R⁹ and R¹⁰ according to the invention are in Table P35 below, wherein each line of lines P35-1 to P35-305 corresponds to one particular embodiment of the invention, wherein P35-1 to P35-305 are also in any combination with one another a preferred embodiment of the present invention. The carbon atom, to which R⁹ bound is marked with * in the drawings and the carbon atom, to which R¹⁰ is bound is marked with # in the drawings. cPr stands for cyclopropyl.

TABLE P35

| line | R⁹ | R¹⁰ |
|---|---|---|
| P35-1 | H | H |
| P35-2 | H | F |
| P35-3 | H | Cl |
| P35-4 | H | Br |
| P35-5 | H | CH₃ |
| P35-6 | H | CH₂CH₃ |
| P35-7 | H | CH₂F |
| P35-8 | H | CHF₂ |
| P35-9 | H | CF₃ |
| P35-10 | H | OCH₃ |
| P35-11 | H | OCH₂F |
| P35-12 | H | OCHF₂ |
| P35-13 | H | OCF₃ |
| P35-14 | H | cPr |
| P35-15 | H | C≡CH |
| P35-16 | H | CN |
| P35-17 | H | S—CH₃ |
| P35-18 | F | H |
| P35-19 | F | F |
| P35-20 | F | Cl |
| P35-21 | F | Br |
| P35-22 | F | CH₃ |
| P35-23 | F | CH₂CH₃ |
| P35-24 | F | CH₂F |
| P35-25 | F | CHF₂ |
| P35-26 | F | CF₃ |
| P35-27 | F | OCH₃ |
| P35-28 | F | OCH₂F |
| P35-29 | F | OCHF₂ |
| P35-30 | F | OCF₃ |
| P35-31 | F | cPr |
| P35-32 | F | C≡CH |
| P35-33 | F | CN |
| P35-34 | F | S—CH₃ |
| P35-35 | Cl | H |
| P35-36 | Cl | F |
| P35-37 | Cl | Cl |
| P35-38 | Cl | Br |
| P35-39 | Cl | CH₃ |
| P35-40 | Cl | CH₂CH₃ |
| P35-41 | Cl | CH₂F |
| P35-42 | Cl | CHF₂ |
| P35-43 | Cl | CF₃ |
| P35-44 | Cl | OCH₃ |
| P35-45 | Cl | OCH₂F |
| P35-46 | Cl | OCHF₂ |
| P35-47 | Cl | OCF₃ |
| P35-48 | Cl | cPr |
| P35-49 | Cl | C≡CH |
| P35-50 | Cl | CN |
| P35-51 | Cl | S—CH₃ |
| P35-52 | Br | H |
| P35-53 | Br | F |
| P35-54 | Br | Cl |
| P35-55 | Br | Br |
| P35-56 | Br | CH₃ |
| P35-57 | Br | CH₂CH₃ |
| P35-58 | Br | CH₂F |
| P35-59 | Br | CHF₂ |
| P35-60 | Br | CF₃ |
| P35-61 | Br | OCH₃ |
| P35-62 | Br | OCH₂F |
| P35-63 | Br | OCHF₂ |
| P35-64 | Br | OCF₃ |
| P35-65 | Br | cPr |
| P35-66 | Br | C≡CH |
| P35-67 | Br | CN |
| P35-68 | Br | S—CH₃ |
| P35-69 | CH₃ | H |
| P35-70 | CH₃ | F |
| P35-71 | CH₃ | Cl |
| P35-72 | CH₃ | Br |
| P35-73 | CH₃ | CH₃ |
| P35-74 | CH₃ | CH₂CH₃ |
| P35-75 | CH₃ | CH₂F |
| P35-76 | CH₃ | CHF₂ |
| P35-77 | CH₃ | CF₃ |
| P35-78 | CH₃ | OCH₃ |
| P35-79 | CH₃ | OCH₂F |
| P35-80 | CH₃ | OCHF₂ |
| P35-81 | CH₃ | OCF₃ |
| P35-82 | CH₃ | cPr |
| P35-83 | CH₃ | C≡CH |
| P35-84 | CH₃ | CN |
| P35-85 | CH₃ | S—CH₃ |
| P35-86 | CH₂CH₃ | H |
| P35-87 | CH₂CH₃ | F |
| P35-88 | CH₂CH₃ | Cl |
| P35-89 | CH₂CH₃ | Br |
| P35-90 | CH₂CH₃ | CH₃ |
| P35-91 | CH₂CH₃ | CH₂CH₃ |
| P35-92 | CH₂CH₃ | CH₂F |
| P35-93 | CH₂CH₃ | CHF₂ |
| P35-94 | CH₂CH₃ | CF₃ |
| P35-95 | CH₂CH₃ | OCH₃ |
| P35-96 | CH₂CH₃ | OCH₂F |
| P35-97 | CH₂CH₃ | OCHF₂ |
| P35-98 | CH₂CH₃ | OCF₃ |
| P35-99 | CH₂CH₃ | cPr |
| P35-100 | CH₂CH₃ | C≡CH |
| P35-101 | CH₂CH₃ | CN |
| P35-102 | CH₂CH₃ | S—CH₃ |
| P35-103 | CH₂F | H |
| P35-104 | CH₂F | F |
| P35-105 | CH₂F | Cl |
| P35-106 | CH₂F | Br |
| P35-107 | CH₂F | CH₃ |
| P35-108 | CH₂F | CH₂CH₃ |
| P35-109 | CH₂F | CH₂F |
| P35-110 | CH₂F | CHF₂ |
| P35-111 | CH₂F | CF₃ |
| P35-112 | CH₂F | OCH₃ |
| P35-113 | CH₂F | OCH₂F |
| P35-114 | CH₂F | OCHF₂ |
| P35-115 | CH₂F | OCF₃ |
| P35-116 | CH₂F | cPr |
| P35-117 | CH₂F | C≡CH |
| P35-118 | CH₂F | CN |
| P35-119 | CH₂F | S—CH₃ |
| P35-120 | CHF₂ | H |
| P35-121 | CHF₂ | F |
| P35-122 | CHF₂ | Cl |
| P35-123 | CHF₂ | Br |
| P35-124 | CHF₂ | CH₃ |
| P35-125 | CHF₂ | CH₂CH₃ |
| P35-126 | CHF₂ | CH₂F |
| P35-127 | CHF₂ | CHF₂ |
| P35-128 | CHF₂ | CF₃ |
| P35-129 | CHF₂ | OCH₃ |
| P35-130 | CHF₂ | OCH₂F |
| P35-131 | CHF₂ | OCHF₂ |
| P35-132 | CHF₂ | OCF₃ |
| P35-133 | CHF₂ | cPr |
| P35-134 | CHF₂ | C≡CH |
| P35-135 | CHF₂ | CN |
| P35-136 | CHF₂ | S—CH₃ |
| P35-137 | CF₃ | H |
| P35-138 | CF₃ | F |
| P35-139 | CF₃ | Cl |
| P35-140 | CF₃ | Br |
| P35-141 | CF₃ | CH₃ |
| P35-142 | CF₃ | CH₂CH₃ |
| P35-143 | CF₃ | CH₂F |
| P35-144 | CF₃ | CHF₂ |
| P35-145 | CF₃ | CF₃ |
| P35-146 | CF₃ | OCH₃ |
| P35-147 | CF₃ | OCH₂F |
| P35-148 | CF₃ | OCHF₂ |
| P35-149 | CF₃ | OCF₃ |
| P35-150 | CF₃ | cPr |
| P35-151 | CF₃ | C≡CH |
| P35-152 | CF₃ | CN |
| P35-153 | CF₃ | S—CH₃ |
| P35-154 | OCH₃ | H |
| P35-155 | OCH₃ | F |
| P35-156 | OCH₃ | Cl |
| P35-157 | OCH₃ | Br |
| P35-158 | OCH₃ | CH₃ |
| P35-159 | OCH₃ | CH₂CH₃ |
| P35-160 | OCH₃ | CH₂F |
| P35-161 | OCH₃ | CHF₂ |
| P35-162 | OCH₃ | CF₃ |
| P35-163 | OCH₃ | OCH₃ |
| P35-164 | OCH₃ | OCH₂F |
| P35-165 | OCH₃ | OCHF₂ |
| P35-166 | OCH₃ | OCF₃ |
| P35-167 | OCH₃ | cPr |
| P35-168 | OCH₃ | C≡CH |
| P35-169 | OCH₃ | CN |
| P35-170 | OCH₃ | S—CH₃ |
| P35-171 | OCH₂F | H |
| P35-172 | OCH₂F | F |
| P35-173 | OCH₂F | Cl |
| P35-174 | OCH₂F | Br |
| P35-175 | OCH₂F | CH₃ |
| P35-176 | OCH₂F | CH₂CH₃ |
| P35-177 | OCH₂F | CH₂F |
| P35-178 | OCH₂F | CHF₂ |
| P35-179 | OCH₂F | CF₃ |
| P35-180 | OCH₂F | OCH₃ |
| P35-181 | OCH₂F | OCH₂F |
| P35-182 | OCH₂F | OCHF₂ |
| P35-183 | OCH₂F | OCF₃ |
| P35-184 | OCH₂F | cPr |
| P35-185 | OCH₂F | C≡CH |
| P35-186 | OCH₂F | CN |
| P35-187 | OCH₂F | S—CH₃ |
| P35-188 | OCHF₂ | H |
| P35-189 | OCHF₂ | F |
| P35-190 | OCHF₂ | Cl |
| P35-191 | OCHF₂ | Br |
| P35-192 | OCHF₂ | CH₃ |
| P35-193 | OCHF₂ | CH₂CH₃ |
| P35-194 | OCHF₂ | CH₂F |
| P35-195 | OCHF₂ | CHF₂ |
| P35-196 | OCHF₂ | CF₃ |
| P35-197 | OCHF₂ | OCH₃ |
| P35-198 | OCHF₂ | OCH₂F |
| P35-199 | OCHF₂ | OCHF₂ |
| P35-200 | OCHF₂ | OCF₃ |
| P35-201 | OCHF₂ | cPr |
| P35-202 | OCHF₂ | C≡CH |
| P35-203 | OCHF₂ | CN |
| P35-204 | OCHF₂ | S—CH₃ |
| P35-205 | OCF₃ | H |
| P35-206 | OCF₃ | F |
| P35-207 | OCF₃ | Cl |
| P35-208 | OCF₃ | Br |
| P35-209 | OCF₃ | CH₃ |
| P35-210 | OCF₃ | CH₂CH₃ |
| P35-211 | OCF₃ | CH₂F |
| P35-212 | OCF₃ | CHF₂ |
| P35-213 | OCF₃ | CF₃ |
| P35-214 | OCF₃ | OCH₃ |
| P35-215 | OCF₃ | OCH₂F |
| P35-216 | OCF₃ | OCHF₂ |
| P35-217 | OCF₃ | OCF₃ |
| P35-218 | OCF₃ | cPr |
| P35-219 | OCF₃ | C≡CH |
| P35-220 | OCF₃ | CN |
| P35-221 | OCF₃ | S—CH₃ |
| P35-222 | cPr | H |
| P35-223 | cPr | F |
| P35-224 | cPr | Cl |
| P35-225 | cPr | Br |
| P35-226 | cPr | CH₃ |
| P35-227 | cPr | CH₂CH₃ |
| P35-228 | cPr | CH₂F |
| P35-229 | cPr | CHF₂ |
| P35-230 | cPr | CF₃ |
| P35-231 | cPr | OCH₃ |
| P35-232 | cPr | OCH₂F |
| P35-233 | cPr | OCHF₂ |
| P35-234 | cPr | OCF₃ |
| P35-235 | cPr | cPr |
| P35-236 | cPr | C≡CH |
| P35-237 | cPr | CN |
| P35-238 | cPr | S—CH₃ |
| P35-239 | C≡CH | H |
| P35-240 | C≡CH | F |
| P35-241 | C≡CH | Cl |
| P35-242 | C≡CH | Br |
| P35-243 | C≡CH | CH₃ |
| P35-244 | C≡CH | CH₂CH₃ |
| P35-245 | C≡CH | CH₂F |
| P35-246 | C≡CH | CHF₂ |
| P35-247 | C≡CH | CF₃ |
| P35-248 | C≡CH | OCH₃ |
| P35-249 | C≡CH | OCH₂F |
| P35-250 | C≡CH | OCHF₂ |
| P35-251 | C≡CH | OCF₃ |
| P35-252 | C≡CH | cPr |
| P35-253 | C≡CH | C≡CH |
| P35-254 | C≡CH | CN |
| P35-255 | C≡CH | S—CH₃ |
| P35-256 | CN | H |
| P35-257 | CN | F |
| P35-258 | CN | Cl |
| P35-259 | CN | Br |
| P35-260 | CN | CH₃ |
| P35-261 | CN | CH₂CH₃ |
| P35-262 | CN | CH₂F |
| P35-263 | CN | CHF₂ |
| P35-264 | CN | CF₃ |
| P35-265 | CN | OCH₃ |
| P35-266 | CN | OCH₂F |
| P35-267 | CN | OCHF₂ |
| P35-268 | CN | OCF₃ |
| P35-269 | CN | cPr |
| P35-270 | CN | C≡CH |
| P35-271 | CN | CN |
| P35-272 | CN | S—CH₃ |
| P35-273 | S—CH₃ | H |
| P35-274 | S—CH₃ | F |
| P35-275 | S—CH₃ | Cl |
| P35-276 | S—CH₃ | Br |
| P35-277 | S—CH₃ | CH₃ |
| P35-278 | S—CH₃ | CH₂CH₃ |
| P35-279 | S—CH₃ | CH₂F |
| P35-280 | S—CH₃ | CHF₂ |
| P35-281 | S—CH₃ | CF₃ |
| P35-282 | S—CH₃ | OCH₃ |
| P35-283 | S—CH₃ | OCH₂F |
| P35-284 | S—CH₃ | OCHF₂ |
| P35-285 | S—CH₃ | OCF₃ |
| P35-286 | S—CH₃ | cPr |
| P35-287 | S—CH₃ | C≡CH |
| P35-288 | S—CH₃ | CN |
| P35-289 | S—CH₃ | S—CH₃ |

| | |
|---|---|
| P35-290 | 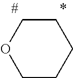 |
| P35-291 | 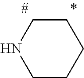 |
| P35-292 | 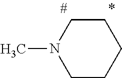 |
| P35-293 | 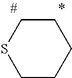 |
| P35-294 | 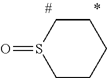 |
| P35-295 | 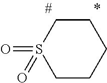 |
| P35-296 | 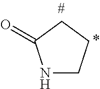 |
| P35-297 | 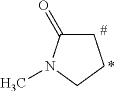 |
| P35-298 | 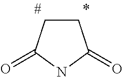 |
| P35-299 | 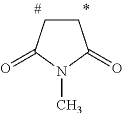 |
| P35-300 | 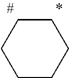 |
| P35-301 | 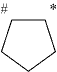 |
| P35-302 | 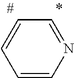 |
| P35-303 | 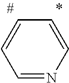 |
| P35-304 | 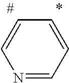 |
| P35-305 | 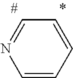 |

R¹² is in each case independently selected from hydrogen, OH, CH(═O), C(═O)C₁-C₆-alkyl, C(═O)C₂-C₆-alkenyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O) NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, O₁—O₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙ-aryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy; wherein n and R^{Y} are as defined above.

R^{12a} is the substituent of the acyclic moieties of R¹². The acyclic moieties of R¹² are not further substituted or carry one, two, three or up to the maximum possible number of identical or different groups R^{12a} which independently of one another are selected from halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, aryl and phenoxy, wherein the heteroaryl, aryl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
R^{12b} is the substituted of carbocyclic, phenyl, heterocyclic and heteroaryl moieties of R¹². The carbocyclic, phenyl, heterocyclic and heteroaryl moieties of R¹² are not further substituted or carry one, two, three, four, five or up to the maximum number of identical or different groups R^{12b} which independently of one another are selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R¹² is H.

According to still another embodiment of formula I, R¹² is OH.

According to a further specific embodiment of formula I, R¹² is CH(═O).

According to a further specific embodiment of formula I, R¹² is C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R¹² is C(═O)C₂-C₆-alkenyl, C(═O)O(C₂-C₆-alkenyl), C(═O)NH(C₂-C₆-alkenyl) or C(═O)N(C₂-C₆-alkenyl)₂), wherein alkenyl is CH═CH₂, CH₂CH═CH₂.

According to a further specific embodiment of formula I, R¹² is C(═O)C₂-C₆-alkynyl, C(═O)O(C₂-C₆-alkynyl), C(═O)NH(C₂-C₆-alkynyl) or C(═O)N(C₂-C₆-alkynyl)₂, wherein alkynyl is C≡CH, CH₂C≡CH.

According to a further specific embodiment of formula I, R¹² is C(═O)C₃-C₆-cycloalkyl, C(═O)O(C₃-C₆-cycloalkyl), C(═O)NH(C₃-C₆-cycloalkyl) or C(═O)N(C₃-C₆-cycloalkyl)₂, wherein cycloalkyl is cyclopropyl (C₃H₇) or cyclobutyl (C₄H₉).

According to a further specific embodiment of formula I, R¹² is CH(═S).

According to a further specific embodiment of formula I, R¹² is C(═S)C₁-C₆-alkyl, C(═S)O(C₁-C₆-alkyl), C(═S)NH(C₁-C₆-alkyl) or C(═S)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R¹² is C(═S)C₂-C₆-alkenyl, C(═S)O(C₂-C₆-alkenyl), C(═S)NH(C₂-C₆-alkenyl) or C(═S)N(C₂-C₆-alkenyl)₂, wherein alkenyl is CH═CH₂, CH₂CH═CH₂.

According to a further specific embodiment of formula I, R¹² is C(═S)O(C₂-C₆-alkynyl), C(═S)NH(C₂-C₆-alkynyl) or C(═S)N(C₂-C₆-alkynyl)₂, wherein alkynyl is C≡CH, CH₂C≡CH.

According to a further specific embodiment of formula I, R¹² is C(═S)C₃-C₆-cycloalkyl, C(═S)O(C₃-C₆-cycloalkyl) or C(═S)N(C₃-C₆-cycloalkyl)₂, wherein cycloalkyl is cyclopropyl (C₃H₇) or cyclobutyl (C₄H₉).

According to still another embodiment of formula I, R¹² is C₁-C₆-alkyl, such as CH₃, C₂H₅, n-propyl, i-propyl, n-butyl, i-butyl, tert-butyl, n-pentyl or i-pentyl.

According to still another embodiment of formula I, R¹² is C₁-C₆-alkyl, in particular C₁-C₄-alkyl, such as CH₃, C₂H₅, n-propyl, i-propyl.

According to still another embodiment of formula I, R¹² is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I R¹² is C₃-C₆-cycloalkyl, in particular cyclopropyl.

According to still another embodiment of formula I, R¹² is C₃-C₆-halogencycloalkyl. In a special embodiment R^{12b} is fully or partially halogenated cyclopropyl, such as 1-F-cyclopropyl, 1-Cl-cyclopropyl, 1,1-F₂-cyclopropyl, 1,1-Cl₂-cyclopropyl.

According to still another embodiment of formula I, R¹² is C₁-C₄-alkoxy and C₁-C₄-halogenalkoxy, in particular C₁-C₃-alkoxy, C₁-C₃-halogenalkoxy, such as CH₂OCH₃, CH₂OCF₃ or CH₂OCHF₂.

According to a further specific embodiment of formula I, R¹² is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;

According to a further specific embodiment of formula I, R¹² is OR^{Y}, wherein R^{Y} is C₁-C₆-alkyl, in particular C₁-C₄-alkyl, more specifically C₁-C₂-alkyl. R¹² is such as OCH₃ or OCH₂CH₃.

According to a further specific embodiment of formula I, R¹² is OR, wherein R^{Y} is C₁-C₆-halogenalkyl, in particular C₁-C₄-halogenalkyl, more specifically C₁-C₂-halogenalkyl. R¹² is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R¹² is OR^{Y}, wherein R^{Y} C₂-C₆-alkenyl, in particular C₂-C₄-alkenyl, more specifically C₁-C₂-alkenyl. R¹² is such as OCH═CH₂, OCH₂CH═CH₂.

According to a further specific embodiment of formula I, R¹² is OR^{Y}, wherein R^{Y} C₂-C₆-alkynyl, in particular C₂-C₆-alkynyl, in particular C₂-C₄-alkynyl, more specifically C₁-C₂-alkynyl. R¹² is such as OC≡CH

According to still another embodiment of formula I, R¹² is OR^{Y}, wherein R^{Y} is C₃-C₆-halogencycloalkyl. In a special embodiment R¹ is fully or partially halogenated cyclopropyl.

According to still another embodiment of formula I, R¹² is is OR^{Y}, wherein R^{Y} and phenyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy.

According to still another embodiment of formula I, R¹² is is OR^{Y}, wherein R^{Y} phenyl-C₁-C₆-alkyl, such as phenyl-CH₂, herein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy. R¹² is such as OCH₂Ph.

According to still a further embodiment of formula I, R¹² is C₂-C₆-alkenyl, in particular C₂-C₄-alkenyl, such as CH═CH₂, C(CH₃)═CH₂, CH₂CH═CH₂.

According to a further specific embodiment of formula I, R¹² is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R¹² is C₂-C₆-alkynyl or C₂-C₆-halogenalkynyl, in particular C₂-C₄-alkynyl or C₂-C₄-halogenalkynyl, such as C≡CH, CH₂C≡CH.

According to still another embodiment of formula I, R¹² is S(O)ₙ—C₁-C₆-alkyl such as SCH₃, S(═O) CH₃, S(O)₂CH₃.

According to still another embodiment of formula I, R¹² is S(O)ₙ—C₁-C₆-halogenalkyl such as SCF₃, S(═O)CF₃, S(O)₂CF₃, SCHF₂, S(═O)CHF₂, S(O)₂CHF₂.

According to still another embodiment of formula I, R¹² is S(O)ₙ-aryl such as S-phenyl, S(═O) phenyl, S(O)₂phenyl, wherein the phenyl group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;

According to still another embodiment of formula I, R¹² is S(O)ₙ—C₂-C₆-alkenyl such as SCH═CH₂, S(═O)CH═CH₂, S(O)₂CH═CH₂, SCH₂CH═CH₂, S(═O)CH₂CH═CH₂, S(O)₂CH₂CH═CH₂.

According to still another embodiment of formula I, R¹² is S(O)ₙ—C₂-C₆-alkynyl such as SC≡CH, S(═O)C≡CH, S(O)₂C≡CH, SCH₂C≡CH, S(═O)CH₂C≡CH, S(O)₂CH₂C≡CH.

According to still another embodiment of formula I, R¹² is SO₂—NH(C₁-C₆-alkyl), is C₁-C₆-alkyl, in particular C₁-C₄-alkyl, more specifically C₁-C₂-alkyl. R¹² is such as SO₂NHCH₃ or SO₂NHCH₂CH₃.

According to still another embodiment of formula I, R¹² is SO₂—NH(C₁-C₆-halogenalkyl), wherein C₁-C₆-halogenalkyl, in particular C₁-C₄-halogenalkyl, more specifically C₁-C₂-halogenalkyl. R¹² is such as SO₂NHCF₃, SO₂NHCHF₂, SO₂NHCH₂F, SO₂NHCCl₃, SO₂NHCHCl₂ or SO₂NHCH₂Cl, in particular SO₂NHCF₃, SO₂NHCHF₂, SO₂NHCCl₃ or SO₂NHCHCl₂.

According to still another embodiment of formula I, R¹² is SO₂—NHaryl, wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy. R¹² is such as SO₂NHPh.

According to still another embodiment of formula I, R¹² is tri-(C₁-C₆ alkyl)silyl, in particular C₁-C₄-alkyl, such as CH₃. or C₂H₅. R¹² is such as OSi(CH₃)₃

According to still another embodiment of formula I, R¹² is di-(C₁-C₆ alkoxy)phosphoryl), in particular C₁-C₄-alkoxy, such as OCH₃. or OC₂H₅. R¹² is such as OPO(OCH₃)₂.

According to still another embodiment of formula I, R¹² is phenyl-C₁-C₆-alkyl, such as phenyl-CH₂, wherein the phenyl moiety in each case is unsubstituted or substituted by one, two or three identical or different groups R^{12b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R¹² is aryl, in particular phenyl, wherein the aryl or phenyl moiety in each case is unsubstituted or substituted by identical or different groups R^{12b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to one embodiment, R¹² is unsubstituted phenyl. According to another embodiment, R¹² is phenyl, that is substituted by one, two or three, in particular one, halogen, in particular selected from F, Cl and Br, more specifically selected from F and Cl.

According to still another embodiment of formula I, R¹² is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R¹² is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R¹² is in each case independently selected from H, halogen, OH, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy and C₃-C₆-cycloalkyl wherein the acyclic moieties of R¹² are unsubstituted or substituted with identical or different groups R^{12a} as defined and preferably defined herein, and wherein the carbocyclic, phenyl and heteroaryl moieties of R¹² are unsubstituted or substituted with identical or different groups R^{12b} as defined and preferably defined herein.

According to still another embodiment of formula I, R¹² is in each case independently selected from H, halogen, OH, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy and C₃-C₆-cycloalkyl, wherein the acyclic moieties of R¹² are unsubstituted or substituted with identical or different groups R^{12a} as defined and preferably defined herein, and wherein the cycloalkyl moieties of R¹² are unsubstituted or substituted with identical or different groups R^{12b} as defined and preferably defined herein.

According to still another embodiment of formula I, R¹² is in each case independently selected from H and OR^{Y}, wherein R^{Y} is most preferably C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy.

According to still another embodiment of formula I, R¹² is in each case independently selected from H and OR^{Y}, wherein R^{Y} is most preferably C₂-C₆-alkenyl, C₂-C₆-alkynyl, phenyl and phenyl-C₁-C₆-alkyl; wherein the phenyl groups are unsubstituted or carry one, two, three, four or five substituents selected from the group consisting of CN, halogen, C₁-C₄-alkyl, C₁-C₄-halogenalkyl, C₁-C₄-alkoxy and C₁-C₄-halogenalkoxy.

According to still another embodiment of formula I, R¹² is in each case independently selected from H, CH(═O), C(═O)C₁-C₆-alkyl, C(═O)O(C₁-C₆-alkyl) and C(═O)NH(C₁-C₆-alkyl), C(═O)N(C₁-C₆-alkyl)₂, C(═O)C₂-C₆-alkenyl, C(═O)O(C₂-C₆-alkenyl), C(═O)NH(C₂-C₆-alkenyl), C(═O)N(C₂-C₆-alkenyl)₂, C(═O)C₂-C₆-alkynyl, C(═O)O(C₂-C₆-alkynyl), C(═O)NH(C₂-C₆-alkynyl), C(═O)N(C₂-C₆-alkynyl)₂C(═O)C₃-C₆-cycloalkyl, C(═O)O(C₃-C₆-cycloalkyl), C(═O)NH(C₃-C₆-cycloalkyl) and C(═O)N(C₃-C₆-cycloalkyl)₂, wherein the acyclic moieties of R¹² are unsubstituted or substituted with identical or different groups R^{12a} as defined and preferably defined herein, and wherein the cycloalkyl moieties of R¹² are unsubstituted or substituted with identical or different groups R^{12b} as defined and preferably defined herein.

According to still another embodiment of formula I, R¹² is in each case independently selected from H, C(═O)C₁-C₆-alkyl, C(═O)O(C₁-C₆-alkyl), C(═O)NH(C₁-C₆-alkyl), C(═O)N(C₁-C₆-alkyl)₂, C(═O)C₂-C₆-alkenyl, C(═O)O(C₂-C₆-alkenyl), C(═O)NH(C₂-C₆-alkenyl), C(═O)N(C₂-C₆-alkenyl)₂, wherein the acyclic moieties of R¹² are unsubstituted or substituted with identical or different groups R^{12a} as defined and preferably defined herein, and wherein the cycloalkyl moieties of R¹² are unsubstituted or substituted with identical or different groups R^{12b} as defined and preferably defined herein.

According to still another embodiment of formula I, R¹² is in each case independently selected from H, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, wherein the acyclic moieties of R¹² are unsubstituted or substituted with identical or different groups R^{12a} as defined and preferably defined herein, and wherein the aryl moieties of R¹² are unsubstituted or substituted with identical or different groups R^{12b} as defined and preferably defined herein.

According to still another embodiment of formula I, R¹² is in each case independently selected from H, SO₂—NH(C₁-C₆-alkyl), SO₂—NH(C₁-C₆-halogenalkyl), SO₂—NHphenyl, wherein the acyclic moieties of R¹² are unsubstituted or substituted with identical or different groups R^{12a} as defined and preferably defined herein, and wherein the aryl moieties of R¹² are unsubstituted or substituted with identical or different groups R^{12b} as defined and preferably defined herein.

According to still another embodiment of formula I, R¹² is in each case independently selected from H, C₁-C₆-alkyl, C(═O)C₁-C₆-alkyl, C(═O)O(C₁-C₆-alkyl), S(O)ₙ—C₁-C₆-alkyl, S(O)ₙaryl, wherein the acyclic moieties of R¹² are unsubstituted or substituted with identical or different groups R^{12a} as defined and preferably defined herein, and wherein the aryl moieties of R¹² are unsubstituted or substituted with identical or different groups R^{12b} as defined and preferably defined herein.

According to still another embodiment of formula I, R¹² is in each case independently selected from H, C(═O)C₁-C₆-alkyl, C(═O)OC₁-C₆-alkyl, C(═O)NHC₁-C₆-alkyl, S(O)₂—C₁-C₆-alkyl, S(O)₂-aryl, SO₂—NH(C₁-C₆-alkyl), OR^{Y}, or C₁-C₄-alkyl; wherein R^{Y} is C₁-C₆-alkyl, C₁-C₆-halogenalkyl, C₂-C₆-alkenyl or C₂-C₆-alkynyl.

According to one embodiment R^{12a} is independently selected from halogen, C₁-C₆-alkoxy, C₃-C₆-cycloalkyl, C₃-C₆-halogencycloalkyl and C₁-C₄-halogenalkoxy. Specifically, R^{12a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R^{12a} is independently halogen, in particular selected from F, Cl, Br and I, more specifically F, Cl and Br.

R^{12b} are the possible substituents for the cycloalkyl, heteroaryl and phenyl moieties of R¹². R^{12b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{12b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{12b} is independently selected from F, Cl, CN, CH₃, CHF₂, CF₃OCH₃ and halogenmethoxy.

Particularly preferred embodiments of R¹² according to the invention are in Table P12 below, wherein each line of lines P12-1 to P12-50 corresponds to one particular embodiment of the invention, wherein P12-1 to P12-50 are also in any combination with one another a preferred embodiment of the present invention. The connection point to the carbon atom, to which R¹² is bound is marked with “#” in the drawings.

TABLE P12

| No. | R¹² |
|---|---|
| P12-1 | H |
| P12-2 | CH₃ |
| P12-3 | CH₂F |
| P12-4 | CHF₂ |
| P12-5 | CF₃ |
| P12-6 | C₂H₅ |
| P12-7 | C₃H₇ |
| P12-8 | CH(CH₃)₂ |
| P12-9 | CH₂CH₂CH₃ |
| P12-10 | CH₂CH₂CH₂CH₃ |
| P12-11 | CH₂CH(CH₃)₂ |
| P12-12 | C(CH₃)₃ |
| P12-13 | CH₂CH₂CH₂CH₂CH₃ |
| P12-14 | CH═CH₂ |
| P12-15 | CH₂CH═CH₂ |
| P12-16 | C≡CH |
| P12-17 | CH₂C≡CH |
| P12-18 | CH₂CH₂CH(CH₃)₂ |
| P12-19 | OH |
| P12-20 | OCH₃ |
| P12-21 | OCHF₂ |
| P12-22 | OC₂H₅ |
| P12-23 | OCH₂OCH₃ |
| P12-24 | OCH₂Ph |
| P12-25 | OCH₂CH═CH₂ |
| P12-26 | C(O)CH₃ |
| P12-27 | C(O)OCH₃ |
| P12-28 | C(O)OCH₂CH₃ |
| P12-29 | C(O)OCH(CH₃)₂ |
| P12-30 | C(O)OC(CH₃)₃ |
| P12-31 | CO—NH₂ |
| P12-32 | CO—NH(CH₃) |
| P12-33 | CO—N(CH₃)₂ |
| P12-34 | SO₂H |
| P12-35 | SO₂—CH₃ |
| P12-36 | SO—CH₃ |
| P12-37 | S—CH₃ |
| P12-38 | SO₂NHCH₃ |
| P12-39 | SO₂NHCF₃ |
| P12-40 | SO₂NHPh |
| P12-41 | SO₂Ph |
| P12-42 | SO₂C₆H₄-4-CH₃ |
| P12-43 | Si(CH₃)₃ |
| P12-44 | PO(OCH₃)₂ |
| P12-45 | 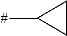 |
| P12-46 | 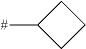 |
| P12-47 | 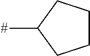 |
| P12-48 | 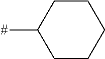 |
| P12-49 | 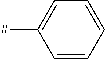 |
| P12-50 | 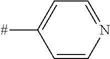 |

Particular embodiments of the compounds I are the following compounds: I-A, I-B, I-C, I-D, I-E, I-F, I-G; II-A, II-B, II-C, II-D, II-E, II-F, II-G; III-A, III-B, III-C, III-D, III-E, III-F, III-G; IV-A, IV-B, IV-C, IV-D, IV-E, IV-F, IV-G. In these formulae, the substituents R⁴, R⁹, R¹⁰ and R¹² are independently as defined in claim 1 or preferably defined below:

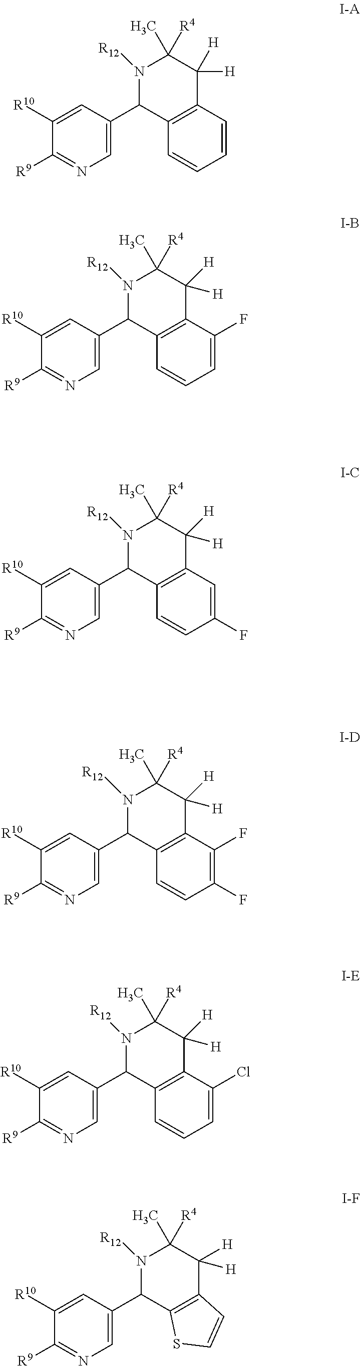

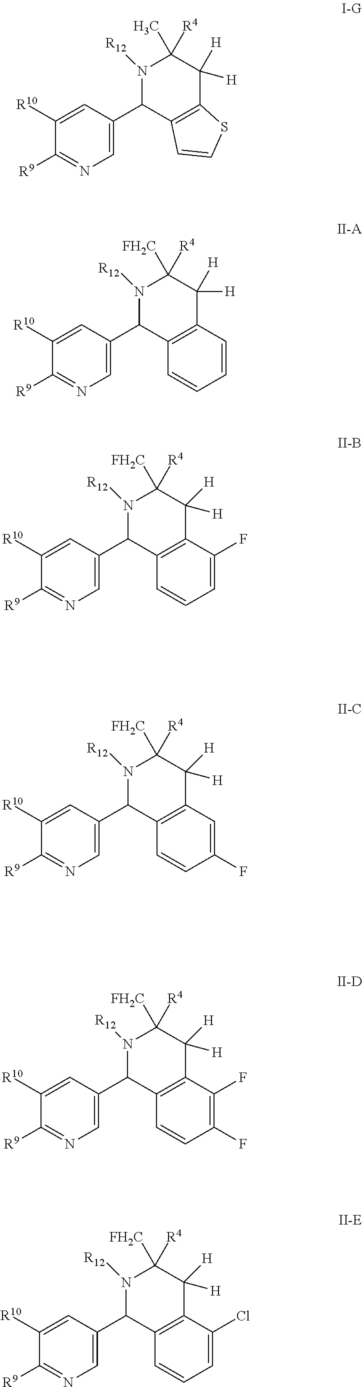

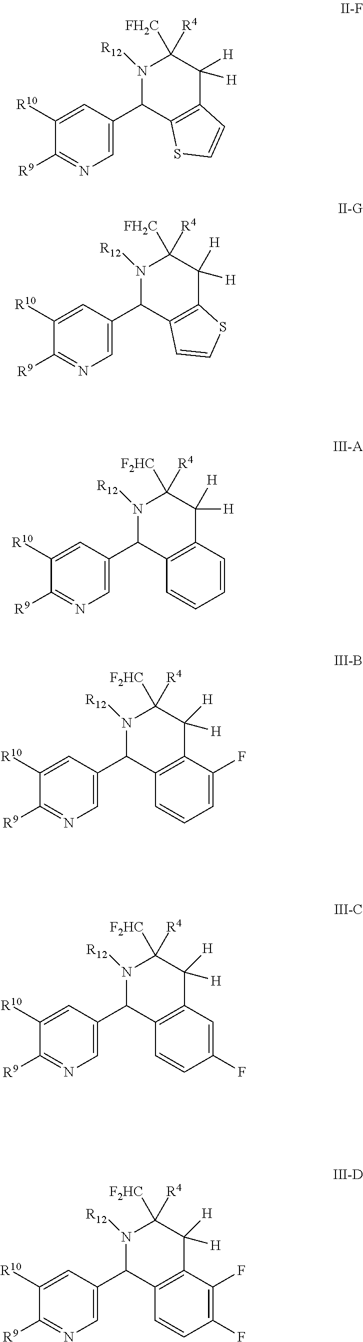

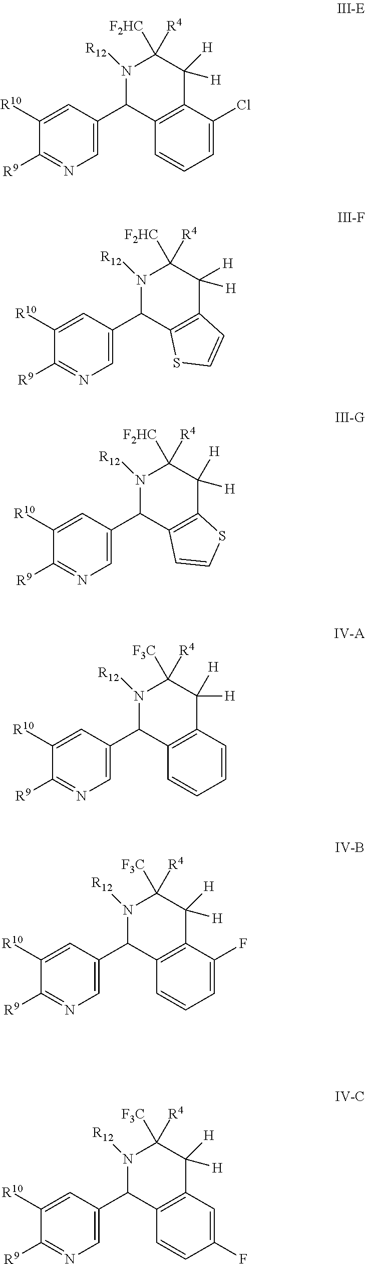

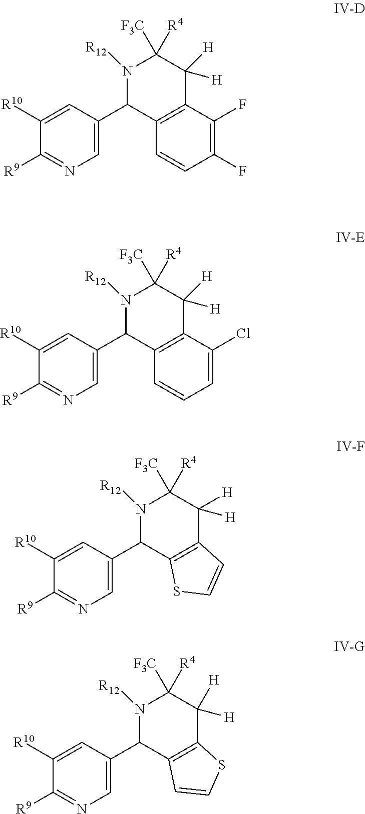

Table 1-1 Compounds of the formula I-A, I-B, I-C, I-D, I-E, I-F, I-G in which R¹² is H and the meaning for the combination of R⁴, R⁹ and R¹⁰ for each individual compound corresponds in each case to one line of Table A (compounds I-A.1-1.A-1 to I-A.1-1.A-550, I-B.1-1.A-1 to I-B.1-1.A-550, I-C.1-1.A-1 to I-C.1-1.A-550, I-D.1-1.A-1 to I-D.1-1.A-550, I-E.1-1.A-1 to I-E.1-1.A-550, I-F.1-1.A-1 to I-F.1-1.A-550, I-G.1-1.A-1 to I-G.1-1.A-550).

Table 1-2 Compounds of the formula I-A, I-B, I-C, I-D, I-E, I-F, I-G in which R¹² is CH₃ and the meaning for the combination of R⁴, R⁹ and R¹⁰ for each individual compound corresponds in each case to one line of Table A (compounds I-A.1-2.A-1 to I-A.1-2.A-550, I-B.1-2.A-1 to I-B.1-2.A-550, I-C.1-2.A-1 to I-C.1-2.A-550, I-D.1-2.A-1 to I-D.1-2.A-550, I-E.1-2.A-1 to I-E.1-2.A-550, I-F.1-2.A-1 to I-F.1-2.A-550, I-G.1-2.A-1 to I-G.1-2.A-550).

Table 1-3 Compounds of the formula I-A, I-B, I-C, I-D, I-E, I-F, I-G in which R¹² is CH₂CH═CH₂ and the meaning for the combination of R⁴, R⁹ and R¹⁰ for each individual compound corresponds in each case to one line of Table A (compounds I-A.1-3.A-1 to I-A.1-3.A-550, I-B.1-3.A-1 to I-B.1-3.A-550, I-C.1-3.A-1 to I-C.1-3.A-550, I-D.1-3.A-1 to I-D.1-3.A-550, I-E.1-3.A-1 to I-E.1-3.A-550, I-F.1-3.A-1 to I-F.1-3.A-550, I-G.1-3.A-1 to I-G.1-3.A-550).

Table 1-4 Compounds of the formula I-A, I-B, I-C, I-D, I-E, I-F, I-G in which R¹² is C(═O)OCH₃ and the meaning for the combination of R⁴, R⁹ and R¹⁰ for each individual compound corresponds in each case to one line of Table A (compounds I-A.1-4.A-1 to I-A.1-4.A-550, I-B.1-4.A-1 to I-B.1-4.A-550, I-C.1-4.A-1 to I-C.1-4.A-550, I-D.1-4.A-1 to I-D.1-4.A-550, I-E.1-4.A-1 to I-E.1-4.A-550, I-F.1-4.A-1 to I-F.1-4.A-550, I-G.1-4.A-1 to I-G.1-4.A-550).

Table 1-5 Compounds of the formula I-A, I-B, I-C, I-D, I-E, I-F, I-G in which R¹² is SO₂NHCH₃ and the meaning for the combination of R⁴, R⁹ and R¹⁰ for each individual compound corresponds in each case to one line of Table A (compounds I-A.1-5.A-1 to I-A.1-5.A-550, I-B.1-5.A-1 to I-B.1-5.A-550, I-C.1-5.A-1 to I-C.1-5.A-550, I-D.1-5.A-1 to I-D.1-5.A-550, I-E.1-5.A-1 to I-E.1-5.A-550, I-F.1-5.A-1 to I-F.1-5.A-550, I-G.1-5.A-1 to I-G.1-5.A-550).

Table 2-1 Compounds of the formula II-A, II-B, II-C, II-D, II-E, II-F, II-G in which R¹² is H and the meaning for the combination of R⁴, R⁹ and R¹⁰ for each individual compound corresponds in each case to one line of Table A (compounds II-A.2-1.A-1 to II-A.2-1.A-550, II-B.2-1.A-1 to II-B.2-1.A-550, II-C.2-1.A-1 to II-C.2-1.A-550, II-D.2-1.A-1 to II-D.2-1.A-550, II-E.2-1.A-1 to II-E.2-1.A-550, II-F.2-1.A-1 to II-F.2-1.A-550, II-G.2-1.A-1 to II-G.2-1.A-550).

Table 2-2 Compounds of the formula II-A, II-B, II-C, II-D, II-E, II-F, II-G in which R¹² is CH₃ and the meaning for the combination of R⁴, R⁹ and R¹⁰ for each individual compound corresponds in each case to one line of Table A (compounds II-A.2-2.A-1 to II-A.2-2.A-550, II-B.2-2.A-1 to II-B.2-2.A-550, II-C.2-2.A-1 to II-C.2-2.A-550, II-D.2-2.A-1 to II-D.2-2.A-550, II-E.2-2.A-1 to II-E.2-2.A-550, II-F.2-2.A-1 to II-F.2-2.A-550, II-G.2-2.A-1 to II-G.2-2.A-550).

Table 2-3 Compounds of the formula II-A, II-B, II-C, II-D, II-E, II-F, II-G in which R¹² is CH₂CH═CH₂ and the meaning for the combination of R⁴, R⁹ and R¹⁰ for each individual compound corresponds in each case to one line of Table A (compounds II-A.2-3.A-1 to II-A.2-3.A-550, II-B.2-3.A-1 to II-B.2-3.A-550, II-C.2-3.A-1 to II-C.2-3.A-550, II-D.2-3.A-1 to II-D.2-3.A-550, II-E.2-3.A-1 to II-E.2-3.A-550, II-F.2-3.A-1 to II-F.2-3.A-550, II-G.2-3.A-1 to II-G.2-3.A-550).

Table 2-4 Compounds of the formula II-A, II-B, II-C, II-D, II-E, II-F, II-G in which R¹² is C(═O)OCH₃ and the meaning for the combination of R⁴, R⁹ and R¹⁰ for each individual compound corresponds in each case to one line of Table A (compounds II-A.2-4.A-1 to II-A.2-4.A-550, II-B.2-4.A-1 to II-B.2-4.A-550, II-C.2-4.A-1 to II-C.2-4.A-550, II-D.2-4.A-1 to II-D.2-4.A-550, II-E.2-4.A-1 to II-E.2-4.A-550, II-F.2-4.A-1 to II-F.2-4.A-550, II-G.2-4.A-1 to II-G.2-4.A-550).

Table 2-5 Compounds of the formula II-A, II-B, II-C, II-D, II-E, II-F, II-G in which R¹² is SO₂NHCH₃ and the meaning for the combination of R⁴, R⁹ and R¹⁰ for each individual compound corresponds in each case to one line of Table A (compounds II-A.2-5.A-1 to II-A.2-5.A-550, II-B.2-5.A-1 to II-B.2-5.A-550, II-C.2-5.A-1 to II-C.2-5.A-550, II-D.2-5.A-1 to II-D.2-5.A-550, II-E.2-5.A-1 to II-E.2-5.A-550, II-F.2-5.A-1 to II-F.2-5.A-550, II-G.2-5.A-1 to II-G.2-5.A-550).

Table 3-1 Compounds of the formula III-A, III-B, III-C, III-D, III-E, III-F, III-G in which R¹² is H and the meaning for the combination of R⁴, R⁹ and R¹⁰ for each individual compound corresponds in each case to one line of Table A (compounds III-A.3-1.A-1 to III-A.3-1.A-550, III-B.3-1.A-1 to III-B.3-1.A-550, III-C.3-1.A-1 to III-C.3-1.A-550, III-D.3-1.A-1 to III-D.3-1.A-550, III-E.3-1.A-1 to III-E.3-1.A-550, III-F.3-1.A-1 to III-F.3-1.A-550, III-G.3-1.A-1 to III-G.3-1.A-550).

Table 3-2 Compounds of the formula III-A, III-B, III-C, III-D, III-E, III-F, III-G in which R¹² is CH₃ and the meaning for the combination of R⁴, R⁹ and R¹⁰ for each individual compound corresponds in each case to one line of Table A (compounds III-A.3-2.A-1 to III-A.3-2.A-550, III-B.3-2.A-1 to III-B.3-2.A-550, III-C.3-2.A-1 to III-C.3-2.A-550, III-D.3-2.A-1 to III-D.3-2.A-550, III-E.3-2.A-1 to III-E.3-2.A-550, III-F.3-2.A-1 to III-F.3-2.A-550, III-G.3-2.A-1 to III-G.3-2.A-550).

Table 3-3 Compounds of the formula III-A, III-B, III-C, III-D, III-E, III-F, III-G in which R¹² is CH₂CH═CH₂ and the meaning for the combination of R⁴, R⁹ and R¹⁰ for each individual compound corresponds in each case to one line of Table A (compounds III-A.3-3.A-1 to III-A.3-3.A-550, III-B.3-3.A-1 to III-B.3-3.A-550, III-C.3-3.A-1 to III-C.3-3.A-550, III-D.3-3.A-1 to III-D.3-3.A-550, III-E.3-3.A-1 to III-E.3-3.A-550, III-F.3-3.A-1 to III-F.3-3.A-550, III-G.3-3.A-1 to III-G.3-3.A-550).

Table 3-4 Compounds of the formula III-A, III-B, III-C, III-D, III-E, III-F, III-G in which R¹² is C(═O)OCH₃ and the meaning for the combination of R⁴, R⁹ and R¹⁰ for each individual compound corresponds in each case to one line of Table A (compounds III-A.3-4.A-1 to III-A.3-4.A-550, III-B.3-4.A-1 to III-B.3-4.A-550, III-C.3-4.A-1 to III-C.3-4.A-550, III-D.3-4.A-1 to III-D.3-4.A-550, III-E.3-4.A-1 to III-E.3-4.A-550, III-F.3-4.A-1 to III-F.3-4.A-550, III-G.3-4.A-1 to III-G.3-4.A-550).

Table 3-5 Compounds of the formula III-A, III-B, III-C, III-D, III-E, III-F, III-G in which R¹² is SO₂NHCH₃ and the meaning for the combination of R⁴, R⁹ and R¹⁰ for each individual compound corresponds in each case to one line of Table A (compounds III-A.3-5.A-1 to III-A.3-5.A-550, III-B.3-5.A-1 to III-B.3-5.A-550, III-C.3-5.A-1 to III-C.3-5.A-550, III-D.3-5.A-1 to III-D.3-5.A-550, III-E.3-5.A-1 to III-E.3-5.A-550, III-F.3-5.A-1 to III-F.3-5.A-550, III-G.3-5.A-1 to III-G.3-5.A-550).

Table 4-1 Compounds of the formula IV-A, IV-B, IV-C, IV-D, IV-E, IV-F, IV-G in which R¹² is H and the meaning for the combination of R⁴, R⁹ and R¹⁰ for each individual compound corresponds in each case to one line of Table A (compounds IV-A.4-1.A-1 to IV-A.4-1.A-550, IV-B.4-1.A-1 to IV-B.4-1.A-550, IV-C.4-1.A-1 to IV-C.4-1.A-550, IV-D.4-1.A-1 to IV-D.4-1.A-550, IV-E.4-1.A-1 to IV-E.4-1.A-550, IV-F.4-1.A-1 to IV-F.4-1.A-550, IV-G.4-1.A-1 to IV-G.4-1.A-550).

Table 4-2 Compounds of the formula IV-A, IV-B, IV-C, IV-D, IV-E, IV-F, IV-G in which R¹² is CH₃ and the meaning for the combination of R⁴, R⁹ and R¹⁰ for each individual compound corresponds in each case to one line of Table A (compounds IV-A.4-2.A-1 to IV-A.4-2.A-550, IV-B.4-2.A-1 to IV-B.4-2.A-550, IV-C.4-2.A-1 to IV-C.4-2.A-550, IV-D.4-2.A-1 to IV-D.4-2.A-550, IV-E.4-2.A-1 to IV-E.4-2.A-550, IV-F.4-2.A-1 to IV-F.4-2.A-550, IV-G.4-2.A-1 to IV-G.4-2.A-550).

Table 4-3 Compounds of the formula IV-A, IV-B, IV-C, IV-D, IV-E, IV-F, IV-G in which R¹² is CH₂CH═CH₂ and the meaning for the combination of R⁴, R⁹ and R¹⁰ for each individual compound corresponds in each case to one line of Table A (compounds IV-A.4-3.A-1 to IV-A.4-3.A-550, IV-B.4-3.A-1 to IV-B.4-3.A-550, IV-C.4-3.A-1 to IV-C.4-3.A-550, IV-D.4-3.A-1 to IV-D.4-3.A-550, IV-E.4-3.A-1 to IV-E.4-3.A-550, IV-F.4-3.A-1 to IV-F.4-3.A-550, IV-G.4-3.A-1 to IV-G.4-3.A-550).

Table 4-4 Compounds of the formula IV-A, IV-B, IV-C, IV-D, IV-E, IV-F, IV-G in which R¹² is C(═O)OCH₃ and the meaning for the combination of R⁴, R⁹ and R¹⁰ for each individual compound corresponds in each case to one line of Table A (compounds IV-A.4-4.A-1 to IV-A.4-4.A-550, IV-B.4-4.A-1 to IV-B.4-4.A-550, IV-C.4-4.A-1 to IV-C.4-4.A-550, IV-D.4-4.A-1 to IV-D.4-4.A-550, IV-E.4-4.A-1 to IV-E.4-4.A-550, IV-F.4-4.A-1 to IV-F.4-4.A-550, IV-G.4-4.A-1 to IV-G.4-4.A-550).

Table 4-5 Compounds of the formula IV-A, IV-B, IV-C, IV-D, IV-E, IV-F, IV-G in which R¹² is SO₂NHCH₃ and the meaning for the combination of R⁴, R⁹ and R¹⁰ for each individual compound corresponds in each case to one line of Table A (compounds IV-A.4-5.A-1 to IV-A.4-5.A-550, IV-B.4-5.A-1 to IV-B.4-5.A-550, IV-C.4-5.A-1 to IV-C.4-5.A-550, IV-D.4-5.A-1 to IV-D.4-5.A-550, IV-E.4-5.A-1 to IV-E.4-5.A-550, IV-F.4-5.A-1 to IV-F.4-5.A-550, IV-G.4-5.A-1 to IV-G.4-5.A-550).

TABLE A

| No. | R⁴ | R⁹ | R¹⁰ |
|---|---|---|---|
| A-1 | CH₂F | CH₃ | CH₃ |
| A-2 | CHF₂ | CH₃ | CH₃ |
| A-3 | CF₃ | CH₃ | CH₃ |
| A-4 | CH₂Cl | CH₃ | CH₃ |
| A-5 | CH₂CH₂Cl | CH₃ | CH₃ |
| A-6 | CN | CH₃ | CH₃ |
| A-7 | CH₂CH₂OCH₃ | CH₃ | CH₃ |
| A-8 | CH═CH₂ | CH₃ | CH₃ |
| A-9 | CCH | CH₃ | CH₃ |
| A-10 | 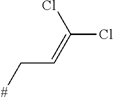 | CH₃ | CH₃ |
| A-11 | 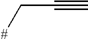 | CH₃ | CH₃ |
| A-12 | C₆H₅ | CH₃ | CH₃ |
| A-13 | —CH₂—C₆H₅ | CH₃ | CH₃ |
| A-14 | 2-py | CH₃ | CH₃ |
| A-15 | 3-py | CH₃ | CH₃ |
| A-16 | 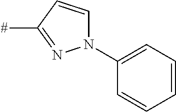 | CH₃ | CH₃ |
| A-17 | 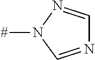 | CH₃ | CH₃ |
| A-18 | 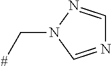 | CH₃ | CH₃ |
| A-19 | 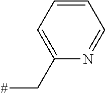 | CH₃ | CH₃ |
| A-20 | 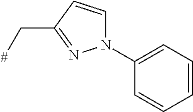 | CH₃ | CH₃ |
| A-21 | 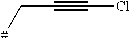 | CH₃ | CH₃ |
| A-22 | 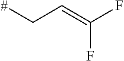 | CH₃ | CH₃ |
| A-23 | CH₂F | CHF₂ | CH₃ |
| A-24 | CHF₂ | CHF₂ | CH₃ |
| A-25 | CF₃ | CHF₂ | CH₃ |
| A-26 | CH₂Cl | CHF₂ | CH₃ |
| A-27 | CH₂CH₂Cl | CHF₂ | CH₃ |
| A-28 | CN | CHF₂ | CH₃ |
| A-29 | CH₂CH₂OCH₃ | CHF₂ | CH₃ |
| A-30 | CH═CH₂ | CHF₂ | CH₃ |
| A-31 | CCH | CHF₂ | CH₃ |
| A-32 | 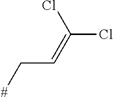 | CHF₂ | CH₃ |
| A-33 | 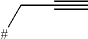 | CHF₂ | CH₃ |
| A-34 | C₆H₅ | CHF₂ | CH₃ |
| A-35 | —CH₂—C₆H₅ | CHF₂ | CH₃ |
| A-36 | 2-py | CHF₂ | CH₃ |
| A-37 | 3-py | CHF₂ | CH₃ |
| A-38 | 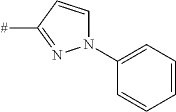 | CHF₂ | CH₃ |
| A-39 | 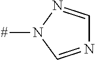 | CHF₂ | CH₃ |
| A-40 | 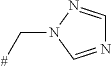 | CHF₂ | CH₃ |
| A-41 | 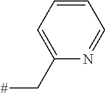 | CHF₂ | CH₃ |
| A-42 | 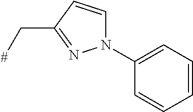 | CHF₂ | CH₃ |
| A-43 | 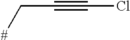 | CHF₂ | CH₃ |
| A-44 | 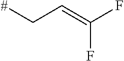 | CHF₂ | CH₃ |
| A-45 | CH₂F | CF₃ | CH₃ |
| A-46 | CHF₂ | CF₃ | CH₃ |
| A-47 | CF₃ | CF₃ | CH₃ |
| A-48 | CH₂Cl | CF₃ | CH₃ |
| A-49 | CH₂CH₂Cl | CF₃ | CH₃ |
| A-50 | CN | CF₃ | CH₃ |
| A-51 | CH₂CH₂OCH₃ | CF₃ | CH₃ |
| A-52 | CH═CH₂ | CF₃ | CH₃ |
| A-53 | CCH | CF₃ | CH₃ |
| A-54 | 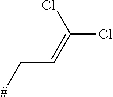 | CF₃ | CH₃ |
| A-55 | 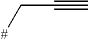 | CF₃ | CH₃ |
| A-56 | C₆H₅ | CF₃ | CH₃ |
| A-57 | —CH₂—C₆H₅ | CF₃ | CH₃ |
| A-58 | 2-py | CF₃ | CH₃ |
| A-59 | 3-py | CF₃ | CH₃ |
| A-60 | 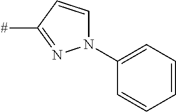 | CF₃ | CH₃ |
| A-61 | 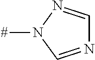 | CF₃ | CH₃ |
| A-62 | 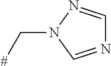 | CF₃ | CH₃ |
| A-63 | 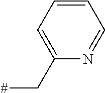 | CF₃ | CH₃ |
| A-64 | 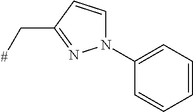 | CF₃ | CH₃ |
| A-65 | 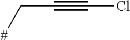 | CF₃ | CH₃ |
| A-66 | 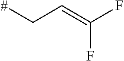 | CF₃ | CH₃ |
| A-67 | CH₂F | Cl | CH₃ |
| A-68 | CHF₂ | Cl | CH₃ |
| A-69 | CF₃ | Cl | CH₃ |
| A-70 | CH₂Cl | Cl | CH₃ |
| A-71 | CH₂CH₂Cl | Cl | CH₃ |
| A-72 | CN | Cl | CH₃ |
| A-73 | CH₂CH₂OCH₃ | Cl | CH₃ |
| A-74 | CH═CH₂ | Cl | CH₃ |
| A-75 | CCH | Cl | CH₃ |
| A-76 | 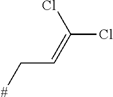 | Cl | CH₃ |
| A-77 | 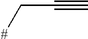 | Cl | CH₃ |
| A-78 | C₆H₅ | Cl | CH₃ |
| A-79 | —CH₂—C₆H₅ | Cl | CH₃ |
| A-80 | 2-py | Cl | CH₃ |
| A-81 | 3-py | Cl | CH₃ |
| A-82 | 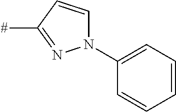 | Cl | CH₃ |
| A-83 | 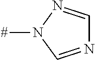 | Cl | CH₃ |
| A-84 | 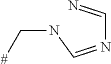 | Cl | CH₃ |
| A-85 | 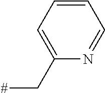 | Cl | CH₃ |
| A-86 | 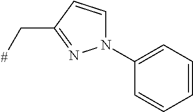 | Cl | CH₃ |
| A-87 | 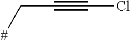 | Cl | CH₃ |
| A-88 | 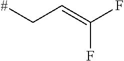 | Cl | CH₃ |
| A-89 | CH₂F | OCH₃ | CH₃ |
| A-90 | CHF₂ | OCH₃ | CH₃ |
| A-91 | CF₃ | OCH₃ | CH₃ |
| A-92 | CH₂Cl | OCH₃ | CH₃ |
| A-93 | CH₂CH₂Cl | OCH₃ | CH₃ |
| A-94 | CN | OCH₃ | CH₃ |
| A-95 | CH₂CH₂OCH₃ | OCH₃ | CH₃ |
| A-96 | CH═CH₂ | OCH₃ | CH₃ |
| A-97 | CCH | OCH₃ | CH₃ |
| A-98 | 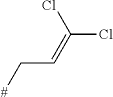 | OCH₃ | CH₃ |
| A-99 | 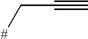 | OCH₃ | CH₃ |
| A-100 | C₆H₅ | OCH₃ | CH₃ |
| A-101 | —CH₂—C₆H₅ | OCH₃ | CH₃ |
| A-102 | 2-py | OCH₃ | CH₃ |
| A-103 | 3-py | OCH₃ | CH₃ |
| A-104 | 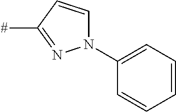 | OCH₃ | CH₃ |
| A-105 | 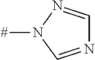 | OCH₃ | CH₃ |
| A-106 | 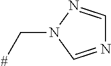 | OCH₃ | CH₃ |
| A-107 | 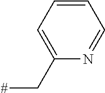 | OCH₃ | CH₃ |
| A-108 | 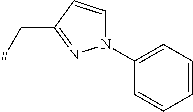 | OCH₃ | CH₃ |
| A-109 | 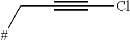 | OCH₃ | CH₃ |
| A-110 | 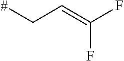 | OCH₃ | CH₃ |
| A-111 | CH₂F | CH₃ | CHF₂ |
| A-112 | CHF₂ | CH₃ | CHF₂ |
| A-113 | CF₃ | CH₃ | CHF₂ |
| A-114 | CH₂Cl | CH₃ | CHF₂ |
| A-115 | CH₂CH₂Cl | CH₃ | CHF₂ |
| A-116 | CN | CH₃ | CHF₂ |
| A-117 | CH₂CH₂OCH₃ | CH₃ | CHF₂ |
| A-118 | CH═CH₂ | CH₃ | CHF₂ |
| A-119 | CCH | CH₃ | CHF₂ |
| A-120 | 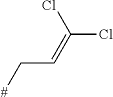 | CH₃ | CHF₂ |
| A-121 | 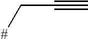 | CH₃ | CHF₂ |
| A-122 | C₆H₅ | CH₃ | CHF₂ |
| A-123 | —CH₂—C₆H₅ | CH₃ | CHF₂ |
| A-124 | 2-py | CH₃ | CHF₂ |
| A-125 | 3-py | CH₃ | CHF₂ |
| A-126 | 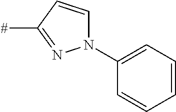 | CH₃ | CHF₂ |
| A-127 | 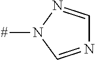 | CH₃ | CHF₂ |
| A-128 | 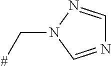 | CH₃ | CHF₂ |
| A-129 | 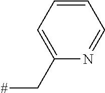 | CH₃ | CHF₂ |
| A-130 | 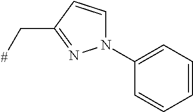 | CH₃ | CHF₂ |
| A-131 | 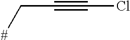 | CH₃ | CHF₂ |
| A-132 | 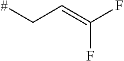 | CH₃ | CHF₂ |
| A-133 | CH₂F | CHF₂ | CHF₂ |
| A-134 | CHF₂ | CHF₂ | CHF₂ |
| A-135 | CF₃ | CHF₂ | CHF₂ |
| A-136 | CH₂Cl | CHF₂ | CHF₂ |
| A-137 | CH₂CH₂Cl | CHF₂ | CHF₂ |
| A-138 | CN | CHF₂ | CHF₂ |
| A-139 | CH₂CH₂OCH₃ | CHF₂ | CHF₂ |
| A-140 | CH═CH₂ | CHF₂ | CHF₂ |
| A-141 | CCH | CHF₂ | CHF₂ |
| A-142 | 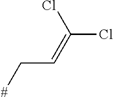 | CHF₂ | CHF₂ |
| A-143 | 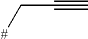 | CHF₂ | CHF₂ |
| A-144 | C₆H₅ | CHF₂ | CHF₂ |
| A-145 | —CH₂—C₆H₅ | CHF₂ | CHF₂ |
| A-146 | 2-py | CHF₂ | CHF₂ |
| A-147 | 3-py | CHF₂ | CHF₂ |
| A-148 | 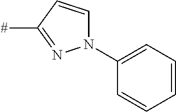 | CHF₂ | CHF₂ |
| A-149 | 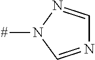 | CHF₂ | CHF₂ |
| A-150 | 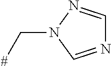 | CHF₂ | CHF₂ |
| A-151 | 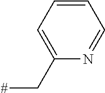 | CHF₂ | CHF₂ |
| A-152 | 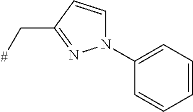 | CHF₂ | CHF₂ |
| A-153 | 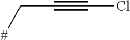 | CHF₂ | CHF₂ |
| A-154 | 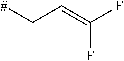 | CHF₂ | CHF₂ |
| A-155 | CH₂F | CF₃ | CHF₂ |
| A-156 | CHF₂ | CF₃ | CHF₂ |
| A-157 | CF₃ | CF₃ | CHF₂ |
| A-158 | CH₂Cl | CF₃ | CHF₂ |
| A-159 | CH₂CH₂Cl | CF₃ | CHF₂ |
| A-160 | CN | CF₃ | CHF₂ |
| A-161 | CH₂CH₂OCH₃ | CF₃ | CHF₂ |
| A-162 | CH═CH₂ | CF₃ | CHF₂ |
| A-163 | CCH | CF₃ | CHF₂ |
| A-164 | 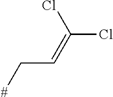 | CF₃ | CHF₂ |
| A-165 | 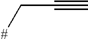 | CF₃ | CHF₂ |
| A-166 | C₆H₅ | CF₃ | CHF₂ |
| A-167 | —CH₂—C₆H₅ | CF₃ | CHF₂ |
| A-168 | 2-py | CF₃ | CHF₂ |
| A-169 | 3-py | CF₃ | CHF₂ |
| A-170 | 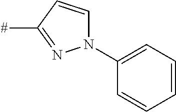 | CF₃ | CHF₂ |
| A-171 | 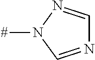 | CF₃ | CHF₂ |
| A-172 | 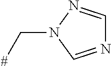 | CF₃ | CHF₂ |
| A-173 | 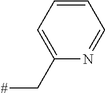 | CF₃ | CHF₂ |
| A-174 | 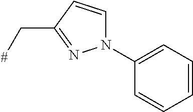 | CF₃ | CHF₂ |
| A-175 | 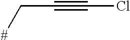 | CF₃ | CHF₂ |
| A-176 | 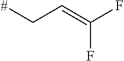 | CF₃ | CHF₂ |
| A-177 | CH₂F | Cl | CHF₂ |
| A-178 | CHF₂ | Cl | CHF₂ |
| A-179 | CF₃ | Cl | CHF₂ |
| A-180 | CH₂Cl | Cl | CHF₂ |
| A-181 | CH₂CH₂Cl | Cl | CHF₂ |
| A-182 | CN | Cl | CHF₂ |
| A-183 | CH₂CH₂OCH₃ | Cl | CHF₂ |
| A-184 | CH═CH₂ | Cl | CHF₂ |
| A-185 | CCH | Cl | CHF₂ |
| A-186 | 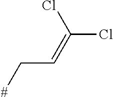 | Cl | CHF₂ |
| A-187 | 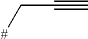 | Cl | CHF₂ |
| A-188 | C₆H₅ | Cl | CHF₂ |
| A-189 | —CH₂—C₆H₅ | Cl | CHF₂ |
| A-190 | 2-py | Cl | CHF₂ |
| A-191 | 3-py | Cl | CHF₂ |
| A-192 | 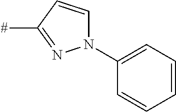 | Cl | CHF₂ |
| A-193 | 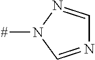 | Cl | CHF₂ |
| A-194 | 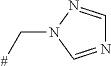 | Cl | CHF₂ |
| A-195 | 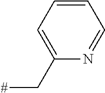 | Cl | CHF₂ |
| A-196 | 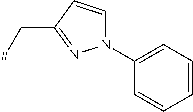 | Cl | CHF₂ |
| A-197 | 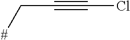 | Cl | CHF₂ |
| A-198 | 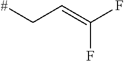 | Cl | CHF₂ |
| A-199 | CH₂F | OCH₃ | CHF₂ |
| A-200 | CHF₂ | OCH₃ | CHF₂ |
| A-201 | CF₃ | OCH₃ | CHF₂ |
| A-202 | CH₂Cl | OCH₃ | CHF₂ |
| A-203 | CH₂CH₂Cl | OCH₃ | CHF₂ |
| A-204 | CN | OCH₃ | CHF₂ |
| A-205 | CH₂CH₂OCH₃ | OCH₃ | CHF₂ |
| A-206 | CH═CH₂ | OCH₃ | CHF₂ |
| A-207 | CCH | OCH₃ | CHF₂ |
| A-208 | 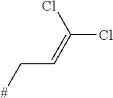 | OCH₃ | CHF₂ |
| A-209 | 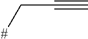 | OCH₃ | CHF₂ |
| A-210 | C₆H₅ | OCH₃ | CHF₂ |
| A-211 | —CH₂—C₆H₅ | OCH₃ | CHF₂ |
| A-212 | 2-py | OCH₃ | CHF₂ |
| A-213 | 3-py | OCH₃ | CHF₂ |
| A-214 | 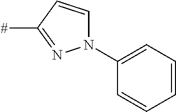 | OCH₃ | CHF₂ |
| A-215 | 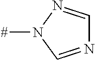 | OCH₃ | CHF₂ |
| A-216 | 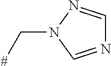 | OCH₃ | CHF₂ |
| A-217 | 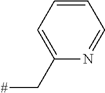 | OCH₃ | CHF₂ |
| A-218 | 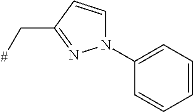 | OCH₃ | CHF₂ |
| A-219 | 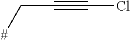 | OCH₃ | CHF₂ |
| A-220 | 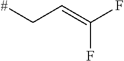 | OCH₃ | CHF₂ |
| A-221 | CH₂F | CH₃ | CF₃ |
| A-222 | CHF₂ | CH₃ | CF₃ |
| A-223 | CF₃ | CH₃ | CF₃ |
| A-224 | CH₂Cl | CH₃ | CF₃ |
| A-225 | CH₂CH₂Cl | CH₃ | CF₃ |
| A-226 | CN | CH₃ | CF₃ |
| A-227 | CH₂CH₂OCH₃ | CH₃ | CF₃ |
| A-228 | CH═CH₂ | CH₃ | CF₃ |
| A-229 | CCH | CH₃ | CF₃ |
| A-230 | 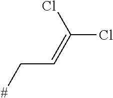 | CH₃ | CF₃ |
| A-231 | 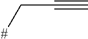 | CH₃ | CF₃ |
| A-232 | C₆H₅ | CH₃ | CF₃ |
| A-233 | —CH₂—C₆H₅ | CH₃ | CF₃ |
| A-234 | 2-py | CH₃ | CF₃ |
| A-235 | 3-py | CH₃ | CF₃ |
| A-236 | 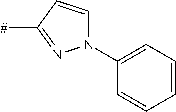 | CH₃ | CF₃ |
| A-237 | 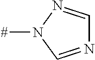 | CH₃ | CF₃ |
| A-238 | 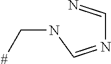 | CH₃ | CF₃ |
| A-239 | 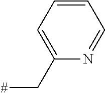 | CH₃ | CF₃ |
| A-240 | 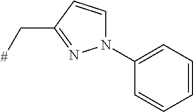 | CH₃ | CF₃ |
| A-241 | 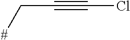 | CH₃ | CF₃ |
| A-242 | 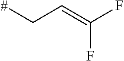 | CH₃ | CF₃ |
| A-243 | CH₂F | CHF₂ | CF₃ |
| A-244 | CHF₂ | CHF₂ | CF₃ |
| A-245 | CF₃ | CHF₂ | CF₃ |
| A-246 | CH₂Cl | CHF₂ | CF₃ |
| A-247 | CH₂CH₂Cl | CHF₂ | CF₃ |
| A-248 | CN | CHF₂ | CF₃ |
| A-249 | CH₂CH₂OCH₃ | CHF₂ | CF₃ |
| A-250 | CH═CH₂ | CHF₂ | CF₃ |
| A-251 | CCH | CHF₂ | CF₃ |
| A-252 | 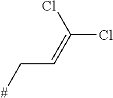 | CHF₂ | CF₃ |
| A-253 | 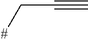 | CHF₂ | CF₃ |
| A-254 | C₆H₅ | CHF₂ | CF₃ |
| A-255 | —CH₂—C₆H₅ | CHF₂ | CF₃ |
| A-256 | 2-py | CHF₂ | CF₃ |
| A-257 | 3-py | CHF₂ | CF₃ |
| A-258 | 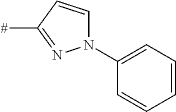 | CHF₂ | CF₃ |
| A-259 | 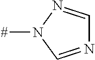 | CHF₂ | CF₃ |
| A-260 | 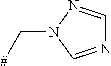 | CHF₂ | CF₃ |
| A-261 | 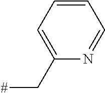 | CHF₂ | CF₃ |
| A-262 | 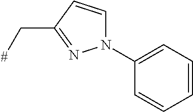 | CHF₂ | CF₃ |
| A-263 | 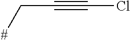 | CHF₂ | CF₃ |
| A-264 | 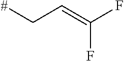 | CHF₂ | CF₃ |
| A-265 | CH₂F | CF₃ | CF₃ |
| A-266 | CHF₂ | CF₃ | CF₃ |
| A-267 | CF₃ | CF₃ | CF₃ |
| A-268 | CH₂Cl | CF₃ | CF₃ |
| A-269 | CH₂CH₂Cl | CF₃ | CF₃ |
| A-270 | CN | CF₃ | CF₃ |
| A-271 | CH₂CH₂OCH₃ | CF₃ | CF₃ |
| A-272 | CH═CH₂ | CF₃ | CF₃ |
| A-273 | CCH | CF₃ | CF₃ |
| A-274 | 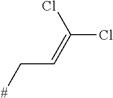 | CF₃ | CF₃ |
| A-275 | 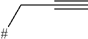 | CF₃ | CF₃ |
| A-276 | C₆H₅ | CF₃ | CF₃ |
| A-277 | —CH₂—C₆H₅ | CF₃ | CF₃ |
| A-278 | 2-py | CF₃ | CF₃ |
| A-279 | 3-py | CF₃ | CF₃ |
| A-280 | 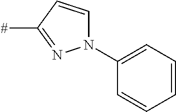 | CF₃ | CF₃ |
| A-281 | 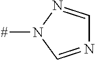 | CF₃ | CF₃ |
| A-282 | 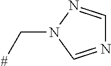 | CF₃ | CF₃ |
| A-283 | 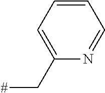 | CF₃ | CF₃ |
| A-284 | 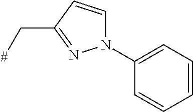 | CF₃ | CF₃ |
| A-285 | 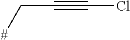 | CF₃ | CF₃ |
| A-286 | 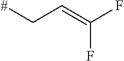 | CF₃ | CF₃ |
| A-287 | CH₂F | Cl | CF₃ |
| A-288 | CHF₂ | Cl | CF₃ |
| A-289 | CF₃ | Cl | CF₃ |
| A-290 | CH₂Cl | Cl | CF₃ |
| A-291 | CH₂CH₂Cl | Cl | CF₃ |
| A-292 | CN | Cl | CF₃ |
| A-293 | CH₂CH₂OCH₃ | Cl | CF₃ |
| A-294 | CH═CH₂ | Cl | CF₃ |
| A-295 | CCH | Cl | CF₃ |
| A-296 | 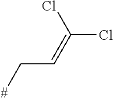 | Cl | CF₃ |
| A-297 | 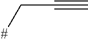 | Cl | CF₃ |
| A-298 | C₆H₅ | Cl | CF₃ |
| A-299 | —CH₂—C₆H₅ | Cl | CF₃ |
| A-300 | 2-py | Cl | CF₃ |
| A-301 | 3-py | Cl | CF₃ |
| A-302 | 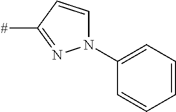 | Cl | CF₃ |
| A-303 | 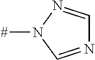 | Cl | CF₃ |
| A-304 | 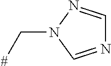 | Cl | CF₃ |
| A-305 | 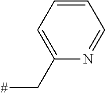 | Cl | CF₃ |
| A-306 | 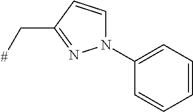 | Cl | CF₃ |
| A-307 | 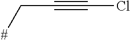 | Cl | CF₃ |
| A-308 | 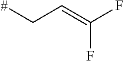 | Cl | CF₃ |
| A-309 | CH₂F | OCH₃ | CF₃ |
| A-310 | CHF₂ | OCH₃ | CF₃ |
| A-311 | CF₃ | OCH₃ | CF₃ |
| A-312 | CH₂Cl | OCH₃ | CF₃ |
| A-313 | CH₂CH₂Cl | OCH₃ | CF₃ |
| A-314 | CN | OCH₃ | CF₃ |
| A-315 | CH₂CH₂OCH₃ | OCH₃ | CF₃ |
| A-316 | CH═CH₂ | OCH₃ | CF₃ |
| A-317 | CCH | OCH₃ | CF₃ |
| A-318 | 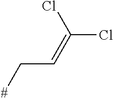 | OCH₃ | CF₃ |
| A-319 | 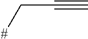 | OCH₃ | CF₃ |
| A-320 | C₆H₅ | OCH₃ | CF₃ |
| A-321 | —CH₂—C₆H₅ | OCH₃ | CF₃ |
| A-322 | 2-py | OCH₃ | CF₃ |
| A-323 | 3-py | OCH₃ | CF₃ |
| A-324 | 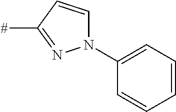 | OCH₃ | CF₃ |
| A-325 | 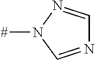 | OCH₃ | CF₃ |
| A-326 | 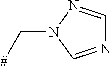 | OCH₃ | CF₃ |
| A-327 | 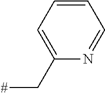 | OCH₃ | CF₃ |
| A-328 | 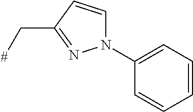 | OCH₃ | CF₃ |
| A-329 | 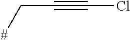 | OCH₃ | CF₃ |
| A-330 | 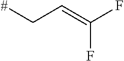 | OCH₃ | CF₃ |
| A-331 | CH₂F | CH₃ | Cl |
| A-332 | CHF₂ | CH₃ | Cl |
| A-333 | CF₃ | CH₃ | Cl |
| A-334 | CH₂Cl | CH₃ | Cl |
| A-335 | CH₂CH₂Cl | CH₃ | Cl |
| A-336 | CN | CH₃ | Cl |
| A-337 | CH₂CH₂OCH₃ | CH₃ | Cl |
| A-338 | CH═CH₂ | CH₃ | Cl |
| A-339 | CCH | CH₃ | Cl |
| A-340 | 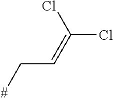 | CH₃ | Cl |
| A-341 | 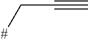 | CH₃ | Cl |
| A-342 | C₆H₅ | CH₃ | Cl |
| A-343 | —CH₂—C₆H₅ | CH₃ | Cl |
| A-344 | 2-py | CH₃ | Cl |
| A-345 | 3-py | CH₃ | Cl |
| A-346 | 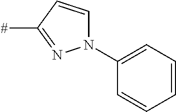 | CH₃ | Cl |
| A-347 | 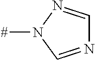 | CH₃ | Cl |
| A-348 | 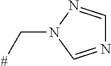 | CH₃ | Cl |
| A-349 | 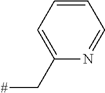 | CH₃ | Cl |
| A-350 | 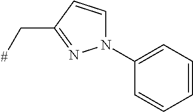 | CH₃ | Cl |
| A-351 | 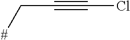 | CH₃ | Cl |
| A-352 | 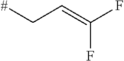 | CH₃ | Cl |
| A-353 | CH₂F | CHF₂ | Cl |
| A-354 | CHF₂ | CHF₂ | Cl |
| A-355 | CF₃ | CHF₂ | Cl |
| A-356 | CH₂Cl | CHF₂ | Cl |
| A-357 | CH₂CH₂Cl | CHF₂ | Cl |
| A-358 | CN | CHF₂ | Cl |
| A-359 | CH₂CH₂OCH₃ | CHF₂ | Cl |
| A-360 | CH═CH₂ | CHF₂ | Cl |
| A-361 | CCH | CHF₂ | Cl |
| A-362 | 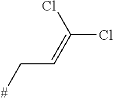 | CHF₂ | Cl |
| A-363 | 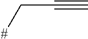 | CHF₂ | Cl |
| A-364 | C₆H₅ | CHF₂ | Cl |
| A-365 | —CH₂—C₆H₅ | CHF₂ | Cl |
| A-366 | 2-py | CHF₂ | Cl |
| A-367 | 3-py | CHF₂ | Cl |
| A-368 | 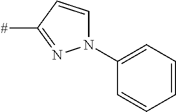 | CHF₂ | Cl |
| A-369 | 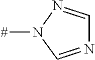 | CHF₂ | Cl |
| A-370 | 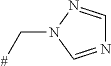 | CHF₂ | Cl |
| A-371 | 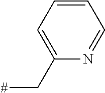 | CHF₂ | Cl |
| A-372 | 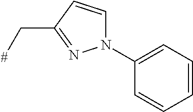 | CHF₂ | Cl |
| A-373 | 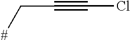 | CHF₂ | Cl |
| A-374 | 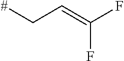 | CHF₂ | Cl |
| A-375 | CH₂F | CF₃ | Cl |
| A-376 | CHF₂ | CF₃ | Cl |
| A-377 | CF₃ | CF₃ | Cl |
| A-378 | CH₂Cl | CF₃ | Cl |
| A-379 | CH₂CH₂Cl | CF₃ | Cl |
| A-380 | CN | CF₃ | Cl |
| A-381 | CH₂CH₂OCH₃ | CF₃ | Cl |
| A-382 | CH═CH₂ | CF₃ | Cl |
| A-383 | CCH | CF₃ | Cl |
| A-384 | 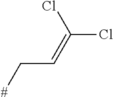 | CF₃ | Cl |
| A-385 | 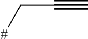 | CF₃ | Cl |
| A-386 | C₆H₅ | CF₃ | Cl |
| A-387 | —CH₂—C₆H₅ | CF₃ | Cl |
| A-388 | 2-py | CF₃ | Cl |
| A-389 | 3-py | CF₃ | Cl |
| A-390 | 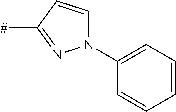 | CF₃ | Cl |
| A-391 | 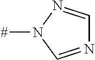 | CF₃ | Cl |
| A-392 | 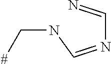 | CF₃ | Cl |
| A-393 | 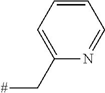 | CF₃ | Cl |
| A-394 | 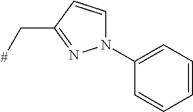 | CF₃ | Cl |
| A-395 | 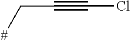 | CF₃ | Cl |
| A-396 | 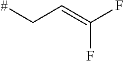 | CF₃ | Cl |
| A-397 | CH₂F | Cl | Cl |
| A-398 | CHF₂ | Cl | Cl |
| A-399 | CF₃ | Cl | Cl |
| A-400 | CH₂Cl | Cl | Cl |
| A-401 | CH₂CH₂Cl | Cl | Cl |
| A-402 | CN | Cl | Cl |
| A-403 | CH₂CH₂OCH₃ | Cl | Cl |
| A-404 | CH═CH₂ | Cl | Cl |
| A-405 | CCH | Cl | Cl |
| A-406 | 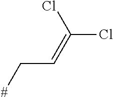 | Cl | Cl |
| A-407 | 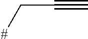 | Cl | Cl |
| A-408 | C₆H₅ | Cl | Cl |
| A-409 | —CH₂—C₆H₅ | Cl | Cl |
| A-410 | 2-py | Cl | Cl |
| A-411 | 3-py | Cl | Cl |
| A-412 | 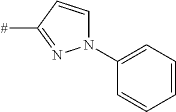 | Cl | Cl |
| A-413 | 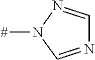 | Cl | Cl |
| A-414 | 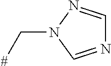 | Cl | Cl |
| A-415 | 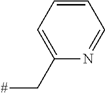 | Cl | Cl |
| A-416 | 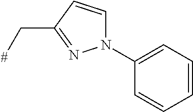 | Cl | Cl |
| A-417 | 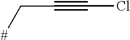 | Cl | Cl |
| A-418 | 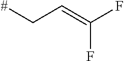 | Cl | Cl |
| A-419 | CH₂F | OCH₃ | Cl |
| A-420 | CHF₂ | OCH₃ | Cl |
| A-421 | CF₃ | OCH₃ | Cl |
| A-422 | CH₂Cl | OCH₃ | Cl |
| A-423 | CH₂CH₂Cl | OCH₃ | Cl |
| A-424 | CN | OCH₃ | Cl |
| A-425 | CH₂CH₂OCH₃ | OCH₃ | Cl |
| A-426 | CH═CH₂ | OCH₃ | Cl |
| A-427 | CCH | OCH₃ | Cl |
| A-428 | 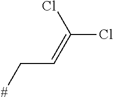 | OCH₃ | Cl |
| A-429 | 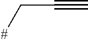 | OCH₃ | Cl |
| A-430 | C₆H₅ | OCH₃ | Cl |
| A-431 | —CH₂—C₆H₅ | OCH₃ | Cl |
| A-432 | 2-py | OCH₃ | Cl |
| A-433 | 3-py | OCH₃ | Cl |
| A-434 | 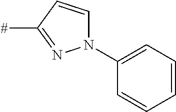 | OCH₃ | Cl |
| A-435 | 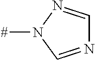 | OCH₃ | Cl |
| A-436 | 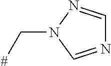 | OCH₃ | Cl |
| A-437 | 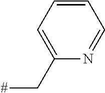 | OCH₃ | Cl |
| A-438 | 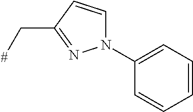 | OCH₃ | Cl |
| A-439 | 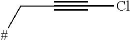 | OCH₃ | Cl |
| A-440 | 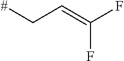 | OCH₃ | Cl |
| A-441 | CH₂F | CH₃ | OCH₃ |
| A-442 | CHF₂ | CH₃ | OCH₃ |
| A-443 | CF₃ | CH₃ | OCH₃ |
| A-444 | CH₂Cl | CH₃ | OCH₃ |
| A-445 | CH₂CH₂Cl | CH₃ | OCH₃ |
| A-446 | CN | CH₃ | OCH₃ |
| A-447 | CH₂CH₂OCH₃ | CH₃ | OCH₃ |
| A-448 | CH═CH₂ | CH₃ | OCH₃ |
| A-449 | CCH | CH₃ | OCH₃ |
| A-450 | 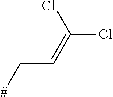 | CH₃ | OCH₃ |
| A-451 | 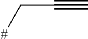 | CH₃ | OCH₃ |
| A-452 | C₆H₅ | CH₃ | OCH₃ |
| A-453 | —CH₂—C₆H₅ | CH₃ | OCH₃ |
| A-454 | 2-py | CH₃ | OCH₃ |
| A-455 | 3-py | CH₃ | OCH₃ |
| A-456 | 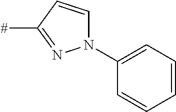 | CH₃ | OCH₃ |
| A-457 | 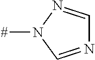 | CH₃ | OCH₃ |
| A-458 | 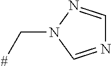 | CH₃ | OCH₃ |
| A-459 | 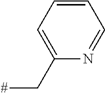 | CH₃ | OCH₃ |
| A-460 | 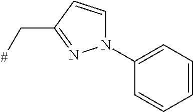 | CH₃ | OCH₃ |
| A-461 | 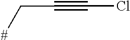 | CH₃ | OCH₃ |
| A-462 | 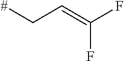 | CH₃ | OCH₃ |
| A-463 | CH₂F | CHF₂ | OCH₃ |
| A-464 | CHF₂ | CHF₂ | OCH₃ |
| A-465 | CF₃ | CHF₂ | OCH₃ |
| A-466 | CH₂Cl | CHF₂ | OCH₃ |
| A-467 | CH₂CH₂Cl | CHF₂ | OCH₃ |
| A-468 | CN | CHF₂ | OCH₃ |
| A-469 | CH₂CH₂OCH₃ | CHF₂ | OCH₃ |
| A-470 | CH═CH₂ | CHF₂ | OCH₃ |
| A-471 | CCH | CHF₂ | OCH₃ |
| A-472 | 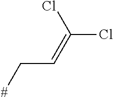 | CHF₂ | OCH₃ |
| A-473 | 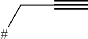 | CHF₂ | OCH₃ |
| A-474 | C₆H₅ | CHF₂ | OCH₃ |
| A-475 | —CH₂—C₆H₅ | CHF₂ | OCH₃ |
| A-476 | 2-py | CHF₂ | OCH₃ |
| A-477 | 3-py | CHF₂ | OCH₃ |
| A-478 | 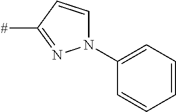 | CHF₂ | OCH₃ |
| A-479 | 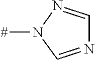 | CHF₂ | OCH₃ |
| A-480 | 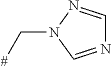 | CHF₂ | OCH₃ |
| A-481 | 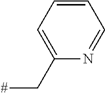 | CHF₂ | OCH₃ |
| A-482 | 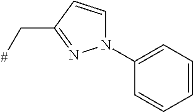 | CHF₂ | OCH₃ |
| A-483 | 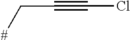 | CHF₂ | OCH₃ |
| A-484 | 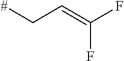 | CHF₂ | OCH₃ |
| A-485 | CH₂F | CF₃ | OCH₃ |
| A-486 | CHF₂ | CF₃ | OCH₃ |
| A-487 | CF₃ | CF₃ | OCH₃ |
| A-488 | CH₂Cl | CF₃ | OCH₃ |
| A-489 | CH₂CH₂Cl | CF₃ | OCH₃ |
| A-490 | CN | CF₃ | OCH₃ |
| A-491 | CH₂CH₂OCH₃ | CF₃ | OCH₃ |
| A-492 | CH═CH₂ | CF₃ | OCH₃ |
| A-493 | CCH | CF₃ | OCH₃ |
| A-494 | 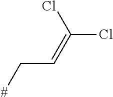 | CF₃ | OCH₃ |
| A-495 | 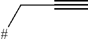 | CF₃ | OCH₃ |
| A-496 | C₆H₅ | CF₃ | OCH₃ |
| A-497 | —CH₂—C₆H₅ | CF₃ | OCH₃ |
| A-498 | 2-py | CF₃ | OCH₃ |
| A-499 | 3-py | CF₃ | OCH₃ |
| A-500 | 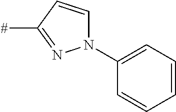 | CF₃ | OCH₃ |
| A-501 | 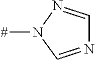 | CF₃ | OCH₃ |
| A-502 | 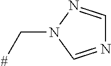 | CF₃ | OCH₃ |
| A-503 | 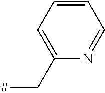 | CF₃ | OCH₃ |
| A-504 | 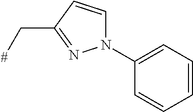 | CF₃ | OCH₃ |
| A-505 | 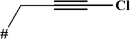 | CF₃ | OCH₃ |
| A-506 | 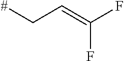 | CF₃ | OCH₃ |
| A-507 | CH₂F | Cl | OCH₃ |
| A-508 | CHF₂ | Cl | OCH₃ |
| A-509 | CF₃ | Cl | OCH₃ |
| A-510 | CH₂Cl | Cl | OCH₃ |
| A-511 | CH₂CH₂Cl | Cl | OCH₃ |
| A-512 | CN | Cl | OCH₃ |
| A-513 | CH₂CH₂OCH₃ | Cl | OCH₃ |
| A-514 | CH═CH₂ | Cl | OCH₃ |
| A-515 | CCH | Cl | OCH₃ |
| A-516 | 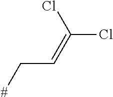 | Cl | OCH₃ |
| A-517 | 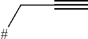 | Cl | OCH₃ |
| A-518 | C₆H₅ | Cl | OCH₃ |
| A-519 | —CH₂—C₆H₅ | Cl | OCH₃ |
| A-520 | 2-py | Cl | OCH₃ |
| A-521 | 3-py | Cl | OCH₃ |
| A-522 | 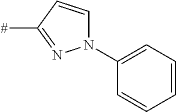 | Cl | OCH₃ |
| A-523 | 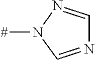 | Cl | OCH₃ |
| A-524 | 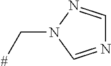 | Cl | OCH₃ |
| A-525 | 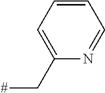 | Cl | OCH₃ |
| A-526 | 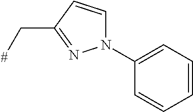 | Cl | OCH₃ |
| A-527 | 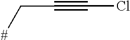 | Cl | OCH₃ |
| A-528 | 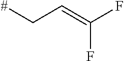 | Cl | OCH₃ |
| A-529 | CH₂F | OCH₃ | OCH₃ |
| A-530 | CHF₂ | OCH₃ | OCH₃ |
| A-531 | CF₃ | OCH₃ | OCH₃ |
| A-532 | CH₂Cl | OCH₃ | OCH₃ |
| A-533 | CH₂CH₂Cl | OCH₃ | OCH₃ |
| A-534 | CN | OCH₃ | OCH₃ |
| A-535 | CH₂CH₂OCH₃ | OCH₃ | OCH₃ |
| A-536 | CH═CH₂ | OCH₃ | OCH₃ |
| A-537 | CCH | OCH₃ | OCH₃ |
| A-538 | 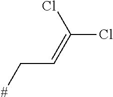 | OCH₃ | OCH₃ |
| A-539 | 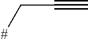 | OCH₃ | OCH₃ |
| A-540 | C₆H₅ | OCH₃ | OCH₃ |
| A-541 | —CH₂—C₆H₅ | OCH₃ | OCH₃ |
| A-542 | 2-py | OCH₃ | OCH₃ |
| A-543 | 3-py | OCH₃ | OCH₃ |
| A-544 | 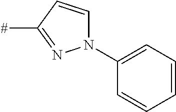 | OCH₃ | OCH₃ |
| A-545 | 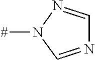 | OCH₃ | OCH₃ |
| A-546 | 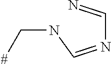 | OCH₃ | OCH₃ |
| A-547 | 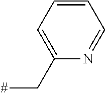 | OCH₃ | OCH₃ |
| A-548 | 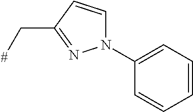 | OCH₃ | OCH₃ |
| A-549 | 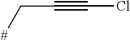 | OCH₃ | OCH₃ |
| A-550 | 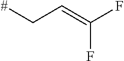 | OCH₃ | OCH₃ |

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as 6-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the Mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici(anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi(Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypi), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuror Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broadleaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii ion sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii(orange rust) on sugar cane and P. asparagion asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani(sheath blight) on rice or R. cerealis (Rhizoctonia spring blight) on wheat or barley; Rhizopus stolonifer(black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. scerotiorum) and soybeans (e. g. S. rolfsiior S. scerotiorum); Septoria spp. on various plants, e. g. S. glycines (brown spot) on soybeans, S. tritici(Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici(syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compounds I and compositions thereof, respectively, are also suitable for controlling harmful fungi in the protection of stored products or harvest and in the protection of materials.

The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Scerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof.

Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible Concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

vi) Water-Dispersible Granules and Water-Soluble Granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable Powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

Biopesticides have been defined as a form of pesticides based on microorganisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classified as microbial pesticides, even though they are multi-cellular.
(2) Biochemical pesticides are naturally occurring substances that control pests or provide other crop protection uses as defined below, but are relatively non-toxic to mammals.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

When living microorganisms, such as microbial pesticides from groups L1), L3) and L5), form part of such kit, it must be taken care that choice and amounts of the components (e. g. chemical pesticides) and of the further auxiliaries should not influence the viability of the microbial pesticides in the composition mixed by the user. Especially for bactericides and solvents, compatibility with the respective microbial pesticide has to be taken into account.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e. g. pesticidally-active substances and biopesticides), in conjunction with which the compounds I can be used, is intended to illustrate the possible combinations but does not limit them:

### A) Respiration Inhibitors

Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenylpyrazol-3-yl)oxylmethyl]phenyl]-N-methoxy-carbamate (A.1.22), 1 [3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methyl pyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methy]phenyl]tetrazol-5-one (A.1.32), (Z,2E)-5-[1-(2,4-dichlorophenyl)pyrazol-3-yl]-oxy-2-methoxyimino-N,3-dimethylpent-3-enamide (A.1.34), (Z,2E)-5-[1-(4-chlorophenyl)pyrazol-3-yl]oxy-2-methoxyimino-N,3-dimethyl-1-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carb y no]-6-methyl-4,9-dioxo-1,5-dioxonan-7-yl] 2-methylpropanoate (A.2.3), fenpicoxamid (A.2.4);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), 3-(difluoromethyl)-1-methyl-N-(1,1,3-trimethylindan-4-yl)pyrazole-4-carboxamide (A.3.22), 3-(trifluoromethyl)-1-methyl-N-(1,1,3-trimethylindan-4-yl)pyrazole-4-carboxamide (A.3.23), 1,3-dimethyl-N-(1,1,3-trimethylindan-4-yl)pyrazole-4-carboxamide (A.3.24), 3-(trifluoromethyl)-1,5-dimethyl-N-(1,1,3-trimethylindan-4-yl)pyrazole-4-carboxamide (A.3.25), 1,3,5-trimethyl-N-(1,1,3-trimethylindan-4-yl)pyrazole-4-carboxamide (A.3.26), 3-(difluoromethyl)-1,5-dimethyl-N-(1,1,3-trimethylindan-4-yl)pyrazole-4-carboxamide (A.3.27), 3-(difluoromethyl)-N-(7-fluoro-1,1,3-trimethyl-indan-4-yl)-1-methyl-pyrazole-4-carboxamide (A.3.28), N-[(5-chloro-2-isopropyl-phenyl)methyl]-N-cyclopropyl-5-fluoro-1,3-dimethyl-pyrazole-4-carboxamide (A.3.29), methyl (E)-2-[2-[(5-cyano-2-methyl-phenoxy)methyl]phenyl]-3-methoxy-prop-2-enoate (A.3.30), N-[(5-chloro-2-isopropyl-phenyl)methyl]-N-cyclopropyl-3-(difluoromethyl)-5-fluoro-1-methyl-pyrazole-4-carboxamide (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), ipfentrifluconazole, (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines and piperazines: fenarimol (B.1.49), pyrifenox (B. 1.50), triforine (B.1.51), [3-(4-chloro-2-fluoro-phenyl)-5-(2,4-difluorophenyl)isoxazol-4-yl]-(3-pyridyl)methanol (B.1.52);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thiabendazole (D.1.4), thiophanate-methyl (D.1.5), 3-chloro-4-(2,6-difluorophenyl)-6-methyl-5-phenyl-pyridazine (D.1.6), 3-chloro-6-methyl-5-phenyl-4-(2,4,6-trifluorophenyl)pyridazine (D.1.7), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);

### E) Inhibitors of Amino Acid and Protein Synthesis

methionine synthesis inhibitors: cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acides: propamocarb (G.4.1);
inhibitors of oxysterol binding protein: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3), 4-[1-[2-[3-(difluoromethyl)-5-methyl-pyrazol-1-yl]acetyl]-4-piperidyl]-N-tetralin-1-ylpyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]-oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K. 1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), 2-[2-[(7,8-difluoro-2-methyl-3-quinolyl)oxy]-6-fluoro-phenyl]propan-2-ol (K.1.44), 2-[2-fluoro-6-[(8-fluoro-2-methyl-3-quinolyl)oxy]phen-yl]propan-2-ol (K.1.45), quinofumelin (K.1.47), 9-fluoro-2,2-dimethyl-5-(3-quinolyl)-3H-1,4-benzoxazepine (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxyphenyl)-N-ethyl-N-methyl-formamidine (K.1.53);

### L) Biopesticides

L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. megaterium, B. mojavensis, B. mycoides, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacterantibioticus, L. enzymogenes, Metschnikowia fructicola, Microdochium dimerum, Microsphaeropsis ochracea, Muscodor albus, Paenibacillus alvei, Paenibacllus polymyxa, Pantoea vagans, Penicllium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperelloides, T. asperellum, T. atroviride, T. fertile, T. gamsil T. harmatum, T. harzianum, T. polysporum, T. stromaticum, T. virens, T. viride, Typhula phacorrhiza, Ulocladium oudemansi Verticillium dahlia, zucchini yellow mosaic virus (avirulent strain);
L2) Biochemical pesticides with fungicidal, bactericidal, viricidal and/or plant defense activator activity: harpin protein, Reynoutria sachalinensis extract;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniarti Burkholderia spp., Chromobacterium subtsugae, Cydia pomonella granulovirus (CpGV), Cryptophlebia leucotreta granulovirus (CrleGV), Flavobacterium spp., Helicoverpa armigera nucleopolyhedrovirus (HearN PV), Helicoverpa zea nucleopolyhedrovirus (HzN PV), Helicoverpa zea single capsid nucleopolyhedrovirus (HzSN PV), Heterorhabditis bacteriophora, Isaria fumosorosea, Lecanicillium longisporum, L. muscarium, Metarhizium anisopliae, Metarhizium anisopliae var. anisopliae, M. anisopliae var. acridum, Nomuraea rileyi, Paecilomyces fumosoroseus, P. liliacinus, Paenibacillus popilliae, Pasteuria spp., P. nishizawae, P. penetrans, P. ramosa, P. thornea, P. usgae, Pseudomonas fluorescens, Spodoptera littoralis nucleopolyhedrovirus (SpliNPV), Steinernema carpocapsae, S. feltiae, S. krausse Streptomyces galbus, S. microflavus;
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, extract of Chenopodium ambrosiodes, Neem oil, Quillay extract;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. laoningense, B. lupini, Delftia acidovorans, Glomus intraradices, Mesorhizobium spp., Rhizobium leguminosarum bv. phaseoli, R. l. bv. trifolii R. l. bv. viciae, R. tropici Sinorhizobium meliloti;

### M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl and uniconazole;

N) Herbicides from Classes N.1 to N.15
N.1 Lipid biosynthesis inhibitors: alloxydim, alloxydim-sodium, butroxydim, clethodim, clodinafop, clodinafop-propargyl, cycloxydim, cyhalofop, cyhalofop-butyl, diclofop, diclofopmethyl, fenoxaprop, fenoxaprop-ethyl, fenoxaprop-P, fenoxaprop-P-ethyl, fluazifop, fluazifopbutyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclo¬propyl-2′-fluoro[1,1′-biphenyl]-3-yl)-5-hydroxy-2,2,6,6-tetramethyl-2H-pyran-3(6H)-one (CAS 1312337-72-6); 4-(2′,4′-dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-dichloro-4-ethyl¬[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;
N.2 ALS inhibitors: amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuronmethyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl, tritosulfuron, imazamethabenz, imazamethabenz-methyl, imazamox, imazapic, imazapyr, imazaquin, imazethapyr; cloransulam, cloransulam-methyl, diclosulam, flumetsulam, florasulam, metosulam, penoxsulam, pyrimisulfan and pyroxsulam; bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methyl¬ethyl ester (CAS 420138-41-6), 4-[[[2-[(4,6-dimethoxy-2-pyrimidinyl)oxy]phenyl]¬methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8); flucarbazone, flucarbazone-sodium, propoxycarbazone, propoxycarbazone-sodium, thiencarbazone, thiencarbazone-methyl; triafamone;
N.3 Photosynthesis inhibitors: amicarbazone; chlorotriazine; ametryn, atrazine, chloridazone, cyanazine, desmetryn, dimethametryn, hexazinone, metribuzin, prometon, prometryn, propazine, simazine, simetryn, terbumeton, terbuthylazin, terbutryn, trietazin; chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron, thiadiazuron, desmedipham, karbutilat, phenmedipham, phenmediphamethyl, bromofenoxim, bromoxynil and its salts and esters, ioxynil and its salts and esters, bromacil, lenacil, terbacil, bentazon, bentazon-sodium, pyridate, pyridafol, pentanochlor, propanil; diquat, diquat-dibromide, paraquat, paraquat-dichloride, paraquat-dimetilsulfate;
N.4 protoporphyrinogen-IX oxidase inhibitors: acifluorfen, acifluorfen-sodium, azafenidin, bencarbazone, benzfendizone, bifenox, butafenacil, carfentrazone, carfentrazone-ethyl, chlormethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6), N-ethyl-3-(2,6-dichloro-4-trifluoro-methylphenoxy)-5-methyl-1H-pyrazole-1-carboxamide (CAS 452098-92-9), N tetrahydrofurfuryl-3-(2,6-dichloro-4-trifluoromethyl phenoxy)-5-methyl-1H-pyrazole-1-carboxamide (CAS 915396-43-9), N-ethyl-3-(2-chloro-6-fluoro-4-trifluoromethyl phenoxy)-5-methyl-1H-pyrazole-1-carboxamide (CAS 452099-05-7), N tetrahydro¬furfuryl-3-(2-chloro-6-fluoro-4-trifluoro¬methylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate (CAS 948893-00-3), 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4);
N.5 Bleacher herbicides: beflubutamid, diflufenican, fluridone, flurochloridone, flurtamone, norflurazon, picolinafen, 4-(3-trifluoromethyl¬phenoxy)-2-(4-trifluoromethylphenyl)¬pyrimidine (CAS 180608-33-7); benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone; aclonifen, amitrole, flumeturon;
N.6 EPSP synthase inhibitors: glyphosate, glyphosate-isopropylammonium, glyposate-potassium, glyphosate-trimesium (sulfosate);
N.7 Glutamine synthase inhibitors: bilanaphos (bialaphos), bilanaphos-sodium, glufosinate, glufosinate-P, glufosinate-ammonium;
N.8 DHP synthase inhibitors: asulam;
N.9 Mitosis inhibitors: benfluralin, butralin, dinitramine, ethalfluralin, fluchloralin, oryzalin, pendimethalin, prodiamine, trifluralin; amiprophos, amiprophos-methyl, butamiphos; chlorthal, chlorthal-dimethyl, dithiopyr, thiazopyr, propyzamide, tebutam; carbetamide, chlorpropham, flamprop, flamprop-isopropyl, flamprop-methyl, flamprop-M-isopropyl, flamprop-M-methyl, propham;
N.10 VLCFA inhibitors: acetochlor, alachlor, butachlor, dimethachlor, dimethenamid, dimethenamid-P, metazachlor, metolachlor, metolachlor-S, pethoxamid, pretilachlor, propachlor, propisochlor, thenylchlor, flufenacet, mefenacet, diphenamid, naproanilide, napropamide, napropamide-M, fentrazamide, anilofos, cafenstrole, fenoxasulfone, ipfencarbazone, piperophos, pyroxasulfone, isoxazoline compounds of the formulae II.1, II.2, II.3, II.4, II.5, II.6, II.7, II.8 and II.9

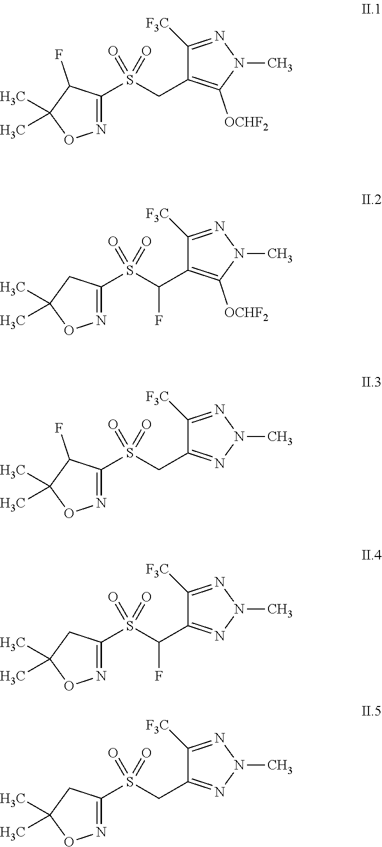

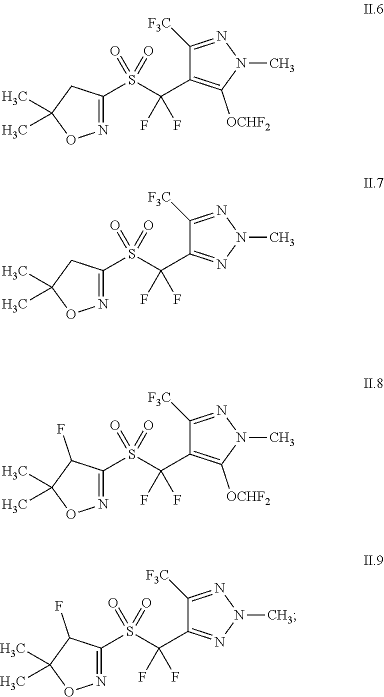

N.11 Cellulose biosynthesis inhibitors: chlorthiamid, dichlobenil, flupoxam, indaziflam, isoxaben, triaziflam, 1-cyclohexyl-5-pentafluorphenyloxy-14-[1,2,4,6]thiatriazin-3-ylamine (CAS 175899-01-1);
N.12 Decoupler herbicides: dinoseb, dinoterb, DNOC and its salts;
N.13 Auxinic herbicides: 2,4-D and its salts and esters, clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (CAS 943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid, benzyl 4-amino-3-chloro-6-(4-chloro-2-fluoro-3-methoxyphenyl)-5-fluoropyridine-2-carboxylate (CAS 1390661-72-9);
N.14 Auxin transport inhibitors: diflufenzopyr, diflufenzopyr-sodium, naptalam and naptalam-sodium;
N.15 Other herbicides: bromobutide, chlorflurenol, chlorflurenol-methyl, cinmethylin, cumyluron, cyclopyrimorate (CAS 499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, tridiphane;
   O) Insecticides from classes O.1 to O.29
O.1 Acetylcholine esterase (AChE) inhibitors: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
O.2 GABA-gated chloride channel antagonists: endosulfan, chlordane; ethiprole, fipronil, flufiprole, pyrafluprole, pyriprole;
O.3 Sodium channel modulators: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alphacypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; DDT, methoxychlor;
O.4 Nicotinic acetylcholine receptor agonists (nAChR): acetamiprid, clothianidin, cycloxaprid, dinotefuran, imidacloprid, nitenpyram, thiacloprid, thiamethoxam; (2E)-1-[(6-chloropyridin-3-yl)methyl]-N′-nitro-2-pentylidenehydrazinecarboximidamide; 1-[(6-chloropyridin-3-yl)methyl]-7-methyl-8-nitro-5-propoxy-1,2,3,5,6,7-hexahydroimidazo[1,2-a]pyridine; nicotine;
O.5 Nicotinic acetylcholine receptor allosteric activators: spinosad, spinetoram;
O.6 Chloride channel activators: abamectin, emamectin benzoate, ivermectin, lepimectin, milbemectin;
O.7 Juvenile hormone mimics: hydroprene, kinoprene, methoprene; fenoxycarb, pyriproxyfen;
O.8 miscellaneous non-specific (multi-site) inhibitors: methyl bromide and other alkyl halides; chloropicrin, sulfuryl fluoride, borax, tartar emetic;
O.9 Selective homopteran feeding blockers: pymetrozine, flonicamid;
O.10 Mite growth inhibitors: clofentezine, hexythiazox, diflovidazin; etoxazole;
O.11 Microbial disruptors of insect midgut membranes: Bacillus thuringiensis, Bacillus sphaericus and the insecticdal proteins they produce: Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, the Bt crop proteins: Cry1Ab, Cry1Ac, Cry1Fa, Cry2Ab, mCry3A, Cry3Ab, Cry3Bb, Cry34/35Ab1;
O.12 Inhibitors of mitochondrial ATP synthase: diafenthiuron; azocyclotin, cyhexatin, fenbutatin oxide, propargite, tetradifon;
O.13 Uncouplers of oxidative phosphorylation via disruption of the proton gradient: chlorfenapyr, DNOC, sulfluramid;
O.14 Nicotinic acetylcholine receptor (nAChR) channel blockers: bensultap, cartap hydrochloride, thiocyclam, thiosultap sodium;
O.15 Inhibitors of the chitin biosynthesis type 0: bistrifluron, chlorfluazuron, diflubenzuron, flucycloxuron, flufenoxuron, hexaflumuron, lufenuron, novaluron, noviflumuron, teflubenzuron, triflumuron;
O.16 Inhibitors of the chitin biosynthesis type 1: buprofezin;
O.17 Moulting disruptors: cyromazine;
O.18 Ecdyson receptor agonists: methoxyfenozide, tebufenozide, halofenozide, fufenozide, chromafenozide;
O.19 Octopamin receptor agonists: amitraz;
O.20 Mitochondrial complex III electron transport inhibitors: hydramethylnon, acequinocyl, fluacrypyrim;
O.21 Mitochondrial complex I electron transport inhibitors: fenazaquin, fenpyroximate, pyrimidifen, pyridaben, tebufenpyrad, tolfenpyrad; rotenone;
O.22 Voltage-dependent sodium channel blockers: indoxacarb, metaflumizone, 2-[2-(4-cyanophenyl)-1-[3-(trifluoromethyl)phenyl]ethylidene]-N-[4-(difluoromethoxy)phenyl]-hydrazinecarboxamide, N-(3-chloro-2-methylphenyl)-2-[(4-chlorophenyl)-[4-[methyl(methylsulfonyl)-amino]phenyl]methylene]-hydrazinecarboxamide;
O.23 Inhibitors of the of acetyl CoA carboxylase: spirodiclofen, spiromesifen, spirotetramat;
O.24 Mitochondrial complex IV electron transport inhibitors: aluminium phosphide, calcium phosphide, phosphine, zinc phosphide, cyanide;
O.25 Mitochondrial complex II electron transport inhibitors: cyenopyrafen, cyflumetofen;
O.26 Ryanodine receptor-modulators: flubendiamide, chlorantraniliprole, cyantraniliprole, cyclaniliprole, tetraniliprole; (R)-3-chloro-N1-{2-methyl-4-[1,2,2,2-tetrafluoro-1-(trifluoromethyl)-ethyl]phenyl}-N2-(1-methyl-2-methylsulfonylethyl)phthalamide, (S)-3-chloro-N1-{2-methyl-4-[1,2,2,2-tetrafluoro-1-(trifluoromethyl)ethyl]phenyl}-N2-(1-methyl-2-methylsulfonylethyl)-phthalamide, methyl-2-[3,5-dibromo-2-({[3-bromo-1-(3-chloropyridin-2-yl)-1H-pyrazol-5-yl]-carbonyl}amino)benzoyl]-1,2-dimethylhydrazinecarboxylate; N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; N-[2-(5-amino-1,3,4-thiadiazol-2-yl)-4-chloro-6-methylphenyl]-3-bromo-1-(3-chloro-2-pyridinyl)-1H-pyrazole-5-carboxamide; 3-chloro-1-(3-chloro-2-pyridinyl)-N-[2,4-dichloro-6-[[(1-cyano-1-methylethyl)amino]carbonyl]phenyl]-1H-pyrazole-5-carboxamide; 3-bromo-N-[2,4-dichloro-6-(methylcarbamoyl)phenyl]-1-(3,5-dichloro-2-pyridyl)-1H-pyrazole-5-carboxamide; N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; cyhalodiamide;
O.27. insecticidal active compounds of unknown or uncertain mode of action: afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, 11-(4-chloro-2,6-dimethylphenyl)-12-hydroxy-1,4-dioxa-9-azadispiro[4.2.4.2]-tetradec-11-en-10-one, 3-(4′-fluoro-2,4-di methyl biphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine, Bacillus firmus; (E/Z)—N-[1-[(6-chloro-3-pyridyl)methyl]-2-pyridylidene]-2,2,2-trifluoro-acetamide; (E/Z)—N-[1-[(6-chloro-5-fluoro-3-pyridyl)methyl]-2-pyridylidene]-2,2,2-trifluoro-acetamide; (E/Z)-2,2,2-trifluoro-N-[1-[(6-fluoro-3-pyridyl)methyl]-2-pyridylidene]acetamide; (E/Z)—N-[1-[(6-bromo-3-pyridyl)methyl]-2-pyridylidene]-2,2,2-trifluoro-acetamide; (E/Z)—N-[1-[1-(6-chloro-3-pyridyl)ethyl]-2-pyridylidene]-2,2,2-trifluoro-acetamide; (E/Z)—N-[1-[(6-chloro-3-pyridyl)methyl]-2-pyridylidene]-2,2-difluoro-acetamide; (E/Z)-2-chloro-N-[1-[(6-chloro-3-pyridyl)methyl]-2-pyridylidene]-2,2-difluoro-acetamide; (E/Z)—N-[1-[(2-chloropyrimidin-5-yl)methyl]-2-pyridylidene]-2,2,2-trifluoro-acetamide; (E/Z)—N-[1-[(6-chloro-3-pyridyl)methyl]-2-pyridylidene]-2,2,3,3,3-pentafluoro-propanamide.); N-[1-[(6-chloro-3-pyridyl)methyl]-2-pyridylidene]-2,2,2-trifluoro-thioacetamide; N-[1-[(6-chloro-3-pyridyl)methyl]-2-pyridylidene]-2,2,2-trifluoro-N′-isopropyl-acetamidine; fluazaindolizine; 4-[5-(3,5-dichlorophenyl)-5-(trifluoromethyl)-4H-isoxazol-3-yl]-2-methyl-N-(1-oxothietan-3-yl)benzamide; fluxametamide; 5-[3-[2,6-dichloro-4-(3,3-dichloroallyloxy)phenoxy]propoxy]-1H-pyrazole; 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide; 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide; N-[3-[[[2-iodo-4-[1,2,2,2-tetrafluoro-1-(trifluoromethyl)ethyl]-6-(trifluoromethyl)phenyl]amino]carbonyl]phenyl]-N-methylbenzamide; N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide; 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; 3-fluoro-N-[2-fluoro-3-[[[2-iodo-4-[1,2,2,2-tetrafluoro-1-(trifluoromethyl)ethyl]-6(trifluoromethyl)phenyl]amino]carbonyl]phenyl]-N-methyl-benzamide; 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide; 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsulfonyl-6-[2-(3-pyridyl)thiazol-5-yl]pyridine-2-carboxamide; N-methylsulfonyl-6-[2-(3-pyridyl)thiazol-5-yl]pyridine-2-carboxamide; N-ethyl-N-[4-methyl-2-(3-pyridyl)thiazol-5-yl]-3-methylthio-propanamide; N-methyl-N-[4-methyl-2-(3-pyridyl)thiazol-5-yl]-3-methylthiopropanamide; N,2-dimethyl-N-[4-methyl-2-(3-pyridyl)thiazol-5-yl]-3-methylthio-propanamide; N-ethyl-2-methyl-N-[4-methyl-2-(3-pyridyl)thiazol-5-yl]-3-methylthio-propanamide; N-[4-chloro-2-(3-pyridyl)thiazol-5-yl]-N-ethyl-2-methyl-3-methylthio-propanamide; N-[4-chloro-2-(3-pyridyl)thiazol-5-yl]-N,2-dimethyl-3-methylthio-propanamide; N-[4-chloro-2-(3-pyridyl)thiazol-5-yl]-N-methyl-3-methylthio-propanamide; N-[4-chloro-2-(3-pyridyl)thiazol-5-yl]-N-ethyl-3-methylthio-propanamide; 1-[(6-chloro-3-pyridinyl)methyl]-1,2,3,5,6,7-hexahydro-5-methoxy-7-methyl-8-nitro-imidazo[1,2-a]pyridine; 1-[(6-chloropyridin-3-yl)methyl]-7-methyl-8-nitro-1,2,3,5,6,7-hexahydroimidazo[1,2-a]pyridin-5-ol; 1-isopropyl-N,5-dimethyl-N-pyridazin-4-yl-pyrazole-4-carboxamide; 1-(1,2-dimethylpropyl)-N-ethyl-5-methyl-N-pyridazin-4-yl-pyrazole-4-carboxamide; N,5-dimethyl-N-pyridazin-4-yl-1-(2,2,2-trifluoro-1-methyl-ethyl)pyrazole-4-carboxamide; 1-[1-(1-cyanocyclopropyl)ethyl]-N-ethyl-5-methyl-N-pyridazin-4-yl-pyrazole-4-carboxamide; N-ethyl-1-(2-fluoro-1-methyl-propyl)-5-methyl-N-pyridazin-4-yl-pyrazole-4-carboxamide; 1-(1,2-dimethylpropyl)-N,5-dimethyl-N-pyridazin-4-yl-pyrazole-4-carboxamide; 1-[1-(1-cyanocyclopropyl)ethyl]-N,5-dimethyl-N-pyridazin-4-yl-pyrazole-4-carboxamide; N-methyl-1-(2-fluoro-1-methyl-propyl]-5-methyl-N-pyridazin-4-yl-pyrazole-4-carboxamide; 1-(4,4-difluorocyclohexyl)-N-ethyl-5-methyl-N-pyridazin-4-yl-pyrazole-4-carboxamide; 1-(4,4-difluorocyclohexyl)-N,5-dimethyl-N-pyridazin-4-yl-pyrazole-4-carboxamide, N-(1-methylethyl)-2-(3-pyridinyl)-2H-indazole-4-carboxamide; N-cyclopropyl-2-(3-pyridinyl)-2H-indazole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tetrahydro-2-furanyl)methyl]-2H-indazole-5-carboxamide; methyl 2-[[2-(3-pyridinyl)-2H-indazol-5-yl]carbonyl]hydrazinecarboxylate; N-[(2,2-difluorocyclopropyl)methyl]-2-(3-pyridinyl)-2H-indazole-5-carboxamide; N-(2,2-difluoropropyl)-2-(3-pyridinyl)-2H-indazole-5-carboxamide; 2-(3-pyridinyl)-N-(2-pyrimidinylmethyl)-2H-indazole-5-carboxamide; N-[(5-methyl-2-pyrazinyl)methyl]-2-(3-pyridinyl)-2H-indazole-5-carboxamide, N-[3-chloro-1-(3-pyridyl)pyrazol-4-yl]-N-ethyl-3-(3,3,3-trifluoropropylsulfanyl)propanamide; N-[3-chloro-1-(3-pyridyl)pyrazol-4-yl]-N-ethyl-3-(3,3,3-trifluoropropylsulfinyl)propanamide; N-[3-chloro-1-(3-pyridyl)pyrazol-4-yl]-3-[(2,2-difluorocyclopropyl)methylsulfanyl]-N-ethyl-propanamide; N-[3-chloro-1-(3-pyridyl)pyrazol-4-yl]-3-[(2,2-difluorocyclopropyl)methylsulfinyl]-N-ethyl-propanamide; sarolaner, lotilaner.

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441).

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In case of a mixture comprising a pesticide II selected from group L), it is preferred that the pesticide II is applied as last treatment.

According to the invention, the solid material (dry matter) of the biopesticides (with the exception of oils such as Neem oil) are considered as active components (e. g. to be obtained after drying or evaporation of the extraction or suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema feltiae.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to further embodiments of the mixtures and compositions, the weight ratio of the component 1) and the component 2) usually is in the range of from 20,000:1 to 1:10, often in the range of from 10,000:1 to 1:1, regularly in the range of from 5,000:1 to 5:1, preferably in the range of from 5,000:1 to 10:1, more preferably in the range of from 2,000:1 to 30:1, even more preferably in the range of from 2,000:1 to 100:1 and in particular in the range of from 1,000:1 to 100:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

According to further embodiments of the mixtures and compositions, the weight ratio of the component 1) and the component 2) usually is in the range of from 10:1 to 1:20,000, often in the range of from 1:1 to 1:10,000, regularly in the range of from 1:5 to 1:5,000, preferably in the range of from 1:10 to 1:5,000, more preferably in the range of from 1:30 to 1:2,000, even more preferably in the range of from 1:100 to 1:2,000 to and in particular in the range of from 1:100 to 1:1,000.

In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

When mixtures comprising microbial pesticides are employed in crop protection, the application rates preferably range from about 1×10⁶ to 5×10¹⁶ (or more) CFU/ha, preferably from about 1×10⁸ to about 1×10¹³ CFU/ha, and even more preferably from about 1×10⁹ to 5×10¹⁵ CFU/ha and particularly preferred even more preferably from 1×10¹² to 5×10¹⁴ CFU/ha. In the case of (entomopathogenic) nematodes as microbial pesticides (e. g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e. g. in the form of eggs, juvenile or any other live stages, preferably in an infective juvenile stage) per ha.

When mixtures comprising microbial pesticides are employed in seed treatment, the application rates with respect to plant propagation material preferably range from about 1×10⁶ to 1×10¹² (or more) CFU/seed. Preferably, the concentration is about 1×10⁶ to about 1×10⁹ CFU/seed. In the case of the microbial pesticides II, the application rates with respect to plant propagation material also preferably range from about 1×10⁷ to 1×10¹⁴ (or more) CFU per 100 kg of seed, preferably from 1×10⁹ to about 1×10¹² CFU per 100 kg of seed.

Preference is also given to mixtures comprising as component 2) at least one active substance selected from inhibitors of complex III at Qₒ site in group A), more preferably selected from compounds (A.1.1), (A.1.4), (A.1.8), (A.1.9), (A.1.10), (A.1.12), (A.1.13), (A.1.14), (A.1.17), (A.1.21), (A.1.24), (A.1.25), (A.1.26), (A.1.27), (A.1.30), (A.1.31), (A.1.32), (A.1.34) and (A.1.35); particularly selected from (A.1.1), (A.1.4), (A.1.8), (A.1.9), (A.1.13), (A.1.14), (A.1.17), (A.1.24), (A.1.25), (A.1.26), (A.1.27), (A.1.30), (A.1.31), (A.1.32), (A.1.34) and (A.1.35).

Preference is also given to mixtures comprising as component 2) at least one active substance selected from inhibitors of complex III at Qᵢ site in group A), more preferably selected from compounds (A.2.1), (A.2.3) and (A.2.4); particularly selected from (A.2.3) and (A.2.4).

Preference is also given to mixtures comprising as component 2) at least one active substance selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.24), (A.3.25), (A.3.27), (A.3.28), (A.3.29), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.12), (A.3.15), (A.3.17), (A.3.19), (A.3.22), (A.3.23), (A.3.24), (A.3.25), (A.3.27), (A.3.29), (A.3.31), (A.3.32), (A.3.33), (A.3.34), (A.3.35), (A.3.36), (A.3.37), (A.3.38) and (A.3.39).

Preference is also given to mixtures comprising as component 2) at least one active substance selected from other respiration inhibitors in group A), more preferably selected from compounds (A.4.5) and (A.4.11); in particular (A.4.11).

Preference is also given to mixtures comprising as component 2) at least one active substance selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.34), (B.1.37), (B.1.38), (B.1.43) and (B.1.46); particularly selected from (B.1.5), (B.1.8), (B.1.10), (B.1.17), (B.1.22), (B.1.23), (B.1.25), (B.1.33), (B.1.34), (B.1.37), (B.138), (B.1.43) and (B.1.46).

Preference is also given to mixtures comprising as component 2) at least one active substance selected from Delta14-reductase inhibitors in group B), more preferably selected from compounds (B.2.4), (B.2.5), (B.2.6) and (B.2.8); in particular (B.2.4).

Preference is also given to mixtures comprising as component 2) at least one active substance selected from phenylamides and acyl amino acid fungicides in group C), more preferably selected from compounds (C.1.1), (C.1.2), (C.1.4) and (C.1.5); particularly selected from (C.1.1) and (C.1.4).

Preference is also given to mixtures comprising as component 2) at least one active substance selected from other nucleic acid synthesis inhibitors in group C), more preferably selected from compounds (C.2.6), (C.2.7) and (C.2.8).

Preference is also given to mixtures comprising as component 2) at least one active substance selected from group D), more preferably selected from compounds (D.1.1), (D.1.2), (D.1.5), (D.2.4) and (D.2.6); particularly selected from (D.1.2), (D.1.5) and (D.2.6).

Preference is also given to mixtures comprising as component 2) at least one active substance selected from group E), more preferably selected from compounds (E.1.1), (E.1.3), (E.2.2) and (E.2.3); in particular (E.1.3).

Preference is also given to mixtures comprising as component 2) at least one active substance selected from group F), more preferably selected from compounds (F.1.2), (F.1.4) and (F.1.5).

Preference is also given to mixtures comprising as component 2) at least one active substance selected from group G), more preferably selected from compounds (G.3.1), (G.3.3), (G.3.6), (G.5.1), (G.5.2), (G.5.3), (G.5.4), (G.5.5), G.5.6), G.5.7), (G.5.8), (G.5.9), (G.5.10) and (G.5.11); particularly selected from (G.3.1), (G.5.1), (G.5.2) and (G.5.3).

Preference is also given to mixtures comprising as component 2) at least one active substance selected from group H), more preferably selected from compounds (H.2.2), (H.2.3), (H.2.5), (H.2.7), (H.2.8), (H.3.2), (H.3.4), (H.3.5), (H.4.9) and (H.4.10); particularly selected from (H.2.2), (H.2.5), (H.3.2), (H.4.9) and (H.4.10).

Preference is also given to mixtures comprising as component 2) at least one active substance selected from group I), more preferably selected from compounds (I.2.2) and (I.2.5).

Preference is also given to mixtures comprising as component 2) at least one active substance selected from group J), more preferably selected from compounds (J.1.2), (J.1.5), (J.1.8), (J.1.11) and (J.1.12); in particular (J.1.5).

Preference is also given to mixtures comprising as component 2) at least one active substance selected from group K), more preferably selected from compounds (K.1.41), (K.1.42), (K.1.44), (K.1.45), (K.1.47) and (K.1.49); particularly selected from (K.1.41), (K.1.44), (K.1.45), (K.1.47) and (K.1.49).

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) may also have fungicidal, bactericidal, viricidal, plant defense activator, insecticidal, acaricidal, molluscidal, pheromone and/or nematicidal activity.

Many of these biopesticides have been deposited under deposition numbers mentioned herein (the prefices such as ATCC or DSM refer to the acronym of the respective culture collection, for details see e. g. here: http://www.wfcc.info/ccinfo/collection/by_acronym/), are referred to in literature, registered and/or are commercially available: mixtures of Aureobasidium pullulans DSM 14940 and DSM 14941 isolated in 1989 in Konstanz, Germany (e. g. blastospores in BlossomProtect® from bio-ferm GmbH, Austria), Azospirillum brasilense Sp245 originally isolated in wheat region of South Brazil (Passo Fundo) at least prior to 1980 (BR 11005; e. g. GELFIX® Gramineas from BASF Agricultural Specialties Ltd., Brazil), A. brasilense strains Ab-V5 and Ab-V6 (e. g. in AzoMax from Novozymes BioAg Produtos papra Agricultura Ltda., Quattro Barras, Brazil or Simbiose-Maiz® from Simbiose-Agro, Brazil; Plant Soil 331, 413-425, 2010), Bacillus amyloliquefaciens strain AP-188 (NRRL B-50615 and B-50331; U.S. Pat. No. 8,445,255); B. amyloliquefaciens spp. plantarum D747 isolated from air in Kikugawa-shi, Japan (US 20130236522 A1; FERM BP-8234; e. g. Double Nickel™ 55 WDG from Certis LLC, USA), B. amyloliquefaciens spp. plantarum FZB24 isolated from soil in Brandenburg, Germany (also called SB3615; DSM 96-2; J. Plant Dis. Prot. 105, 181-197, 1998; e. g. Taegro® from Novozyme Biologicals, Inc., USA), B. amyloliquefaciens ssp. plantarum FZB42 isolated from soil in Brandenburg, Germany (DSM 23117; J. Plant Dis. Prot. 105, 181-197, 1998; e. g. RhizoVital® 42 from AbiTEP GmbH, Germany), B. amyloliquefaciens ssp. plantarum MBI600 isolated from faba bean in Sutton Bonington, Nottinghamshire, U.K. at least before 1988 (also called 1430; NRRL B-50595; US 2012/0149571 A1; e. g. Integral® from BASF Corp., USA), B. amyloliquefaciens spp. plantarum QST-713 isolated from peach orchard in 1995 in California, U.S.A. (NRRL B-21661; e. g. Serenade® MAX from Bayer Crop Science LP, USA), B. amyloliquefaciens spp. plantarumTJ1000 isolated in 1992 in South Dakoda, U.S.A. (also called 1 BE; ATCC BAA-390; CA 2471555 A1; e. g. QuickRoots™ from TJ Technologies, Watertown, S. Dak., USA), B. firmus CNCM I-1582, a variant of parental strain EIP-N1 (CNCM I-1556) isolated from soil of central plain area of Israel (WO 2009/126473, U.S. Pat. No. 6,406,690; e. g. Votivo® from Bayer CropScience LP, USA), B. pumilus GHA 180 isolated from apple tree rhizosphere in Mexico (IDAC 260707-01; e. g. PRO-MIX® BX from Premier Horticulture, Quebec, Canada), B. pumilus INR-7 otherwise referred to as BU-F22 and BU-F33 isolated at least before 1993 from cucumber infested by Erwinia tracheiphila (NRRL B-50185, NRRL B-50153; U.S. Pat. No. 8,445,255), B. pumilus KFP9F isolated from the rhizosphere of grasses in South Africa at least before 2008 (NRRL B-50754; WO 2014/029697; e. g. BAC-UP or FUSION-P from BASF Agricultural Specialities (Pty) Ltd., South Africa), B. pumilus QST 2808 was isolated from soil collected in Pohnpei, Federated States of Micronesia, in 1998 (NRRL B-30087; e. g. Sonata® or Ballad® Plus from Bayer Crop Science LP, USA), B. simplex ABU 288 (NRRL B-50304; U.S. Pat. No. 8,445,255), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. thuringiensis ssp. aizawai ABTS-1857 isolated from soil taken from a lawn in Ephraim, Wis., U.S.A., in 1987 (also called ABG-6346; ATCC SD-1372; e. g. XenTari® from BioFa AG, Monsingen, Germany), B. t. ssp. kurstaki ABTS-351 identical to HD-1 isolated in 1967 from diseased Pink Bollworm black larvae in Brownsville, Tex., U.S.A. (ATCC SD-1275; e. g. Dipel® DF from Valent BioSciences, IL, USA), B. t. ssp. kurstaki SB4 isolated from E. saccharina larval cadavers (NRRL B-50753; e. g. Beta Pro® from BASF Agricultural Specialities (Pty) Ltd., South Africa), B. t. ssp. tenebrionis NB-176-1, a mutant of strain NB-125, a wild type strain isolated in 1982 from a dead pupa of the beetle Tenebrio molitor (DSM 5480; EP 585 215 B1; e. g. Novodor® from Valent BioSciences, Switzerland), Beauveria bassiana GHA (ATCC 74250; e. g. BotaniGard® 22WGP from Laverlam Int. Corp., USA), B. bassiana JW-1 (ATCC 74040; e. g. Naturalis® from CBC (Europe) S.r.l., Italy), B. bassiana PPRI 5339 isolated from the larva of the tortoise beetle Conchyloctenia punctata (NRRL 50757; e. g. BroadBand® from BASF Agricultural Specialities (Pty) Ltd., South Africa), Bradyrhizobium elkanii strains SEMIA 5019 (also called 29W) isolated in Rio de Janeiro, Brazil and SEMIA 587 isolated in 1967 in the State of Rio Grande do Sul, from an area previously inoculated with a North American isolate, and used in commercial inoculants since 1968 (Appl. Environ. Microbiol. 73(8), 2635, 2007; e. g. GELFIX 5 from BASF Agricultural Specialties Ltd., Brazil), B. japonicum 532c isolated from Wisconsin field in U.S.A. (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011); B. japonicum strains deposited at SEMIA known from Appl. Environ. Microbiol. 73(8), 2635, 2007: SEMIA 5079 isolated from soil in Cerrados region, Brazil by Embrapa-Cerrados used in commercial inoculants since 1992 (CPAC 15; e. g. GELFIX 5 or ADHERE 60 from BASF Agricultural Specialties Ltd., Brazil), B. japonicum SEMIA 5080 obtained under lab conditions by Embrapa-Cerrados in Brazil and used in commercial inoculants since 1992, being a natural variant of SEMIA 586 (CB1809) originally isolated in U.S.A. (CPAC 7; e. g. GELFIX 5 or ADHERE 60 from BASF Agricultural Specialties Ltd., Brazil); Burkholderia sp. A396 isolated from soil in Nikko, Japan, in 2008 (NRRL B-50319; WO 2013/032693; Marrone Bio Innovations, Inc., USA), Coniothyrium minitans CON/M/91-08 isolated from oilseed rape (WO 1996/021358; DSM 9660; e. g. Contans® WG, Intercept® WG from Bayer CropScience AG, Germany), harpin (alpha-beta) protein (Science 257, 85-88, 1992; e. g. Messenger™ or HARP-N-Tek from Plant Health Care plc, U.K.), Helicoverpa armigera nucleopolyhedrovirus (HearNPV) (J. Invertebrate Pathol. 107, 112-126, 2011; e. g. Helicovex® from Adermatt Biocontrol, Switzerland; Diplomata® from Koppert, Brazil; Vivus® Max from AgBiTech Pty Ltd., Queensland, Australia), Helicoverpa zea single capsid nucleopolyhedrovirus (HzSNPV) (e. g. Gemstar® from Certis LLC, USA), Helicoverpa zea nucleopolyhedrovirus ABA-NPV-U (e. g. Heligen® from AgBiTech Pty Ltd., Queensland, Australia), Heterorhabditis bacteriophora (e. g. Nemasys® G from BASF Agricultural Specialities Limited, UK), Isaria fumosorosea Apopka-97 isolated from mealy bug on gynura in Apopka, Fla., U.S.A. (ATCC 20874; Biocontrol Science Technol. 22(7), 747-761, 2012; e. g. PFR-97™ or PreFeRal® from Certis LLC, USA), Metarhizium anisopliae var. anisopliae F52 also called 275 or V275 isolated from codling moth in Austria (DSM 3884, ATCC 90448; e. g. Met52® Novozymes Biologicals BioAg Group, Canada), Metschnikowia fructicola 277 isolated from grapes in the central part of Israel (U.S. Pat. No. 6,994,849; NRRL Y-30752; e. g. formerly Shemer® from Agrogreen, Israel), Paeciomyces ilacinus 251 isolated from infected nematode eggs in the Philippines (AGAL 89/030550; WO1991/02051; Crop Protection 27, 352-361, 2008; e. g. BioAct® from Bayer CropScience AG, Germany and MeloCon® from Certis, USA), Paenibacillus alvei NAS6G6 isolated from the rhizosphere of grasses in South Africa at least before 2008 (WO 2014/029697; NRRL B-50755; e.g. BAC-UP from BASF Agricultural Specialities (Pty) Ltd., South Africa), Pasteuria nishizawae Pn1 isolated from a soybean field in the mid-2000s in Illinois, U.S.A. (ATCC SD-5833; Federal Register 76(22), 5808, Feb. 2, 2011; e.g. Clariva™ PN from Syngenta Crop Protection, LLC, USA), Penicillium bilaiae (also called P. bilaii) strains ATCC 18309 (=ATCC 74319), ATCC 20851 and/or ATCC 22348 (=ATCC 74318) originally isolated from soil in Alberta, Canada (Fertilizer Res. 39, 97-103, 1994; Can. J. Plant Sci. 78(1), 91-102, 1998; U.S. Pat. No. 5,026,417,

WO 1995/017806; e. g. Jump Start®, Provide® from Novozymes Biologicals BioAg Group, Canada), Reynoutria sachalinensis extract (EP 0307510 B1; e. g. Regalia® SC from Marrone BioInnovations, Davis, Calif., USA or Milsana® from BioFa AG, Germany), Steinernema carpocapsae (e. g. Millenium® from BASF Agricultural Specialities Limited, UK), S. feltiae (e. g. Nemashield® from BioWorks, Inc., USA; Nemasys® from BASF Agricultural Specialities Limited, UK), Streptomyces microflavus NRRL B-50550 (WO 2014/124369; Bayer CropScience, Germany), Trichoderma asperelloides JM41R isolated in South Africa (NRRL 50759; also referred to as T. fertile; e. g. Trichoplus® from BASF Agricultural Specialities (Pty) Ltd., South Africa), T. harzianum T-22 also called KRL-AG2 (ATCC 20847; BioControl 57, 687-696, 2012; e. g. Plantshield® from BioWorks Inc., USA or SabrEx™ from Advanced Biological Marketing Inc., Van Wert, Ohio, USA).

According to one embodiment of the inventive mixtures, the at least one pesticide II is selected from the groups L1) to L5):
L1) Microbial pesticides with fungicidal, bactericidal, viricidal and/or plant defense activator activity: Aureobasidium pullulans DSM 14940 and DSM 14941 (L1.1), Bacillus amyloliquefaciens AP-188 (L.1.2), B. amyloliquefaciens ssp. plantarum D747 (L.1.3), B. amyloliquefaciens ssp. plantarum FZB24 (L.1.4), B. amyloliquefaciens ssp. plantarum FZB42 (L.1.5), B. amyloliquefaciens ssp. plantarum MBI600 (L. 1.6), B. amyloliquefaciens ssp. plantarum QST-713 (L.1.7), B. amyloliquefaciens ssp. plantarum TJ 1000 (L.1.8), B. pumilus GB34 (L.1.9), B. pumilus GHA 180 (L.1.10), B. pumilus INR-7 (L.1.11), B. pumilus KFP9F (L.1.12), B. pumilus QST 2808 (L.1.13), B. simplex ABU 288 (L.1.14), B. subtilis FB17 (L. 1.15), Coniothyrium minitans CON/M/91-08 (L. 1.16), Metschnikowia fructicola NRRL Y-30752 (L.1.17), Paenibacillus alvei NAS6G6 (L.1.18), Penicillium bilaiae ATCC 22348 (L.1.19), P. bilaiae ATCC 20851 (L.1.20), Penicillium bilaiae ATCC 18309 (L.1.21), Streptomyces microflavus NRRL B-50550 (L.1.22), Trichoderma asperelloides JM41R (L.1.23), T. harzianum T-22 (L.1.24);
L2) Biochemical pesticides with fungicidal, bactericidal, viricidal and/or plant defense activator activity: harpin protein (L.2.1), Reynoutria sachalinensis extract (L.2.2);
L3) Microbial pesticides with insecticidal, acaricidal, molluscidal and/or nematicidal activity: Bacillus firmus I-1582 (L.3.1); B. thuringiensis ssp. aizawai ABTS-1857 (L.3.2), B. t. ssp. kurstaki ABTS-351 (L.3.3), B. t. ssp. kurstaki SB4 (L.3.4), B. t. ssp. tenebrionis NB-176-1 (L.3.5), Beauveria bassiana GHA (L.3.6), B. bassiana JW-1 (L.3.7), B. bassiana PPRI 5339 (L.3.8), Burkholderia sp. A396 (L.3.9), Helicoverpa armigera nucleopolyhedrovirus (HearNPV) (L.3.10), Helicoverpa zea nucleopolyhedrovirus (HzN PV) ABA-NPV-U (L.3.11), Helicoverpa zea single capsid nucleopolyhedrovirus (HzSNPV) (L.3.12), Heterohabditis bacteriophora (L.3.13), Isaria fumosorosea Apopka-97 (L.3.14), Metarhizium anisopliae var. anisopliae F52 (L.3.15), Paecilomyces lilacinus 251 (L.3.16), Pasteuria nishizawae Pn1 (L.3.17), Steinernema carpocapsae (L.3.18), S. feltiae (L.3.19);
L4) Biochemical pesticides with insecticidal, acaricidal, molluscidal, pheromone and/or nematicidal activity: cis-jasmone (L.4.1), methyl jasmonate (L.4.2), Quillay extract (L.4.3);
L5) Microbial pesticides with plant stress reducing, plant growth regulator, plant growth promoting and/or yield enhancing activity: Azospirillum brasilense Ab-V5 and Ab-V6 (L.5.1), A. brasilense Sp245 (L.5.2), Bradyrhizobium elkanii SEMIA 587 (L.5.3), B. elkanii SEMIA 5019 (L.5.4), B. japonicum 532c (L.5.5), B. japonicum E-109 (L.5.6), B. japonicum SEMIA 5079 (L.5.7), B. japonicum SEMIA 5080 (L.5.8).

The present invention furthermore relates to agrochemical compositions comprising a mixture of XXX (component 1) and at least one biopesticide selected from the group L) (component 2), in particular at least one biopesticide selected from the groups L1) and L2), as described above, and if desired at least one suitable auxiliary.

The present invention furthermore relates to agrochemical compositions comprising a mixture of XXX (component 1) and at least one biopesticide selected from the group L) (component 2), in particular at least one biopesticide selected from the groups L3) and L4), as described above, and if desired at least one suitable auxiliary.

Preference is also given to mixtures comprising as pesticide II (component 2) a biopesticide selected from the groups L1), L3) and L5), preferably selected from strains denoted above as (L.1.2), (L.1.3), (L.1.4), (L.1.5), (L.1.6), (L.1.7), (L.1.8), (L.1.10), (L.1.11), (L.1.12), (L.1.13), (L.1.14), (L.1.15), (L.1.17), (L.1.18), (L.1.19), (L.1.20), (L.1.21), (L.3.1); (L.3.9), (L.3.16), (L.3.17), (L.5.1), (L.5.2), (L.5.3), (L.5.4), (L.5.5), (L.5.6), (L.5.7), (L.5.8); (L.4.2), and (L.4.1); even more preferably selected from (L.1.2), (L.1.6), (L.1.7), (L.1.8), (L.1.11), (L.1.12), (L.1.13), (L.1.14), (L.1.15), (L.1.18), (L.1.19), (L.1.20), (L.1.21), (L.3.1); (L.3.9), (L.3.16), (L.3.17), (L.5.1), (L.5.2), (L.5.5), (L.5.6); (L.4.2), and (L.4.1). These mixtures are particularly suitable for treatment of propagation materials, i. e. seed treatment purposes and likewise for soil treatment. These seed treatment mixtures are particularly suitable for crops such as cereals, corn and leguminous plants such as soybean.

Preference is also given to mixtures comprising as pesticide II (component 2) a biopesticide selected from the groups L1), L3) and L5), preferably selected from strains denoted above as (L1.1), (L.1.2), (L.1.3), (L.1.6), (L.1.7), (L.1.9), (L.1.11), (L.1.12), (L.1.13), (L.1.14), (L.1.15), (L.1.17), (L.1.18), (L.1.22), (L.1.23), (L.1.24), (L.2.2); (L.3.2), (L.3.3), (L.3.4), (L.3.5), (L.3.6), (L.3.7), (L.3.8), (L.3.10), (L.3.11), (L.3.12), (L.3.13), (L.3.14), (L.3.15), (L.3.18), (L.3.19); (L.4.2), even more preferably selected from (L.1.2), (L.1.7), (L.1.11), (L.1.13), (L.1.14), (L.1.15), (L.1.18), (L.1.23), (L.3.3), (L.3.4), (L.3.6), (L.3.7), (L.3.8), (L.3.10), (L.3.11), (L.3.12), (L.3.15), and (L.4.2). These mixtures are particularly suitable for foliar treatment. These mixtures for foliar treatment are particularly suitable for vegetables, fruits, vines, cereals, corn, leguminous crops such as soybeans.

The mixtures of active substances can be prepared as compositions comprising besides the active ingredients at least one inert ingredient (auxiliary) by usual means, e. g. by the means given for the compositions of compounds I. Concerning usual ingredients of such compositions reference is made to the explanations given for the compositions containing compounds I.

According to one embodiment, the microbial pesticides selected from groups L1), L3) and L5) embrace not only the isolated, pure cultures of the respective microorganism as defined herein, but also its cell-free extract, its suspensions in a whole broth culture or as a metabolite-containing culture medium or a purified metabolite obtained from a whole broth culture of the microorganism.

When living microorganisms, such as pesticides II from groups L1), L3) and L5), form part of the compositions, such compositions can be prepared as compositions comprising besides the active ingredients at least one auxiliary by usual means (e. g. H. D. Burges: Formulation of Micobial Biopesticides, Springer, 1998). Suitable customary types of such compositions are suspensions, dusts, powders, pastes, granules, pressings, capsules, and mixtures thereof. Examples for composition types are suspensions, capsules, pastes, pastilles, wettable powders or dusts, pressings, granules, insecticidal articles, as well as gel formulations. Herein, it has to be taken into account that each formulation type or choice of auxiliary should not influence the viability of the microorganism during storage of the composition and when finally applied to the soil, plant or plant propagation material. Suitable formulations are e. g. mentioned in WO 2008/002371, U.S. Pat. Nos. 6,955,912, 5,422,107.

### SYNTHESIS EXAMPLE

With due modification of the starting compounds, the procedures shown in the synthesis examples below were used to obtain further compounds I. The resulting compounds, together with physical data, are listed in Table I below.
HPLC-MS: HPLC-column Kinetex XB C18 1.7μ (50×2.1 mm); eluent: acetonitrile/water+0.1% TFA (5 gradient from 5:95 to 100:0 in 1.5 min at 60° C., flow gradient from 0.8 to 1.0 ml/min in 1.5 min). MS: Quadrupol Electrospray Ionisation, 80 V (positive mode).

### Example 1) Synthesis of 3-(3,3-dichloroallyl)-1-[6-(difluoromethyl)-5-methyl-3-pyridyl]-3-methyl-2,4-dihydro-1H-isoquinoline (I-1)

### 1. Synthesis of 2-(2,2-dichlorocyclopropyl)-1-phenyl-propan-2-ol

The mixture of 2,2-dichlorocyclopropyl methyl ketone (2,645 g, 18.8 mmol) in THF (100 mL) was added BnMgCl (56.3 mL, 56.3 mmol) dropwise at 0° C. under N₂, the mixture was stirred for 3 h at 0° C. The reaction mixture was quenched with aq. NH₄Cl (50 mL) and extracted with MTBE (50 mL), dried over Na₂SO₄ and concentrated, the residue was purified by column (PE:EtOAc=40:1) to give the tittle compound (2.7 g, 60%) as yellow oil.

¹H-NMR (CDCl₃, δ in ppm): 1.43-1.54 (m, 3H), 1.58 (dd, J=8.38, 7.06 Hz, 1H), 1.65-1.72 (m, 1H), 2.81-2.93 (m, 2H), 7.13-7.42 (m, 5H).

### 2. Synthesis of [(1E)-5,5-dichloro-2-methyl-penta-1,4-dienyl]benzene

The solution of 2-(2,2-dichlorocyclopropyl)-1-phenyl-propan-2-ol (0.4 g, 1.64 mmol) in toluene (20 mL) was added p-TsOH (156 mg, 0.82 mmol) under N₂, the solution was heated to 80° C. for 16 h. The reaction mixture was concentrated, the residue was purified by column (PE) to give the tittle compound (254 mg, crude) as colorless oil.

### 3. Synthesis of methyl 5-[3-(3,3-dichloroallyl)-3-methyl-4H-isoquinolin-1-yl]-3-methyl-pyridine-2-carboxylate

The solution of methyl 5-cyano-3-methyl-pyridine-2-carboxylate (105 mg, 0.6 mmol) and [(1E)-5,5-dichloro-2-methyl-penta-1,4-dienyl]benzene (270 mg, 1.2 mmol) were in DCM (20 mL) was added TfOH (450 mg, 3 mmol) dropwise at 0° C. under N₂, the mixture was stirred for 1.5 h at 0° C. The reaction mixture was quenched with aq. NaHCO₃ (30 mL) and extracted with DCM (20 mL), the organic layer was dried over Na₂SO₄ and concentrated, the residue was purified by Pre-TLC (PE:EtOAc=1:1) to give the tittle compound (111 mg, 46%) as red oil.

¹H-NMR (CDCl₃, δ in ppm): 1.22 (s, 3H), 2.43-2.58 (m, 2H), 2.66 (s, 3H), 2.73-2.93 (m, 2H), 3.96-4.04 (m, 3H), 6.01-6.09 (m, 1H). 7.14 (d, J=7.50 Hz, 1H). 7.21-7.28 (m, 2H). 7.37-7.46 (m, 1H), 7.85 (s, 1H), 8.69 (s, 1H).

### 4. Synthesis of 5-[3-(3,3-dichloroallyl)-3-methyl-4H-isoquinolin-1-yl]-3-methyl-pyridine-2-carbaldehyde

The mixture of 5-[3-(3,3-dichloroallyl)-3-methyl-4H-isoquinolin-1-yl]-3-methyl-pyridine-2-carboxylic acid (360 mg, 0.89 mmol) in DCM (20 mL) was added DIBAL-H (1.34 mL, 1.34 mmol) dropwise at −78° C. under N₂, the mixture was stirred for 1 h at −78° C. The reaction mixture was quenched with aq. NH₄Cl (30 mL) and extracted with DCM (30 mL), dried over Na₂SO₄ and concentrated to give the tittle compound (360 mg, crude) as yellow solid.

¹H-NMR (CDCl₃, δ in ppm): 2.37 (s, 5H), 2.47-2.60 (m, 3H), 2.74 (s, 3H), 2.77-2.95 (m, 3H), 6.08 (t, J=7.59 Hz, 1H), 7.40-7.48 (m, 1H), 7.85 (s, 1H), 8.83 (s, 1H), 10.27 (s, 1H).

### 5. Synthesis of 3-(3,3-dichloroallyl)-1-[6-(difluoromethyl)-5-methyl-3-pyridyl]-3-methyl-4H-isoquinoline

The solution of 5-[3-(3,3-dichloroallyl)-3-methyl-4H-isoquinolin-1-yl]-3-methyl-pyridine-2-carbaldehyde (0.5 g, 1.34 mmol) in DCM (40 mL) was added DAST (1,534 g, 6.7 mmol) dropwise at 0° C. under N₂, the solution was stirred for 1 h at 0° C. The reaction mixture was quenched with aq. NaHCO₃ (60 mL) and extracted with DCM (30 mL), dried over Na₂SO₄ and concentrated, the residue was purified by Pre-TLC (PE:EtOAc=3:1) to give tittle compound (180 mg, 34%) as colorless oil.

¹H-NMR (CDCl₃, δ in ppm): 1.25 (s, 3H), 2.45-2.55 (m, 2H), 2.6 (s, 3H), 2.75 (d, 1H), 2.95 (d, 1H), 6.05 (t, 1H), 6.61-6.90 (t, 1H), 7.15 (d, 1H), 7.20-7.25 (m, 1H+1H), 7.45 (t, 1H), 7.83 (s, 1H), 8.61 (s, 1H).

*HPLC-MS: Rt=1.097 min; M⁺+H=394.9

### 6. Synthesis of 3-(3,3-dichloroallyl)-1-[6-(difluoromethyl)-5-methyl-3-pyridyl]-3-methyl-2,4-dihydro-1H-isoquinoline

To a solution of 3-(3,3-dichloroallyl)-1-[6-(difluoromethyl)-5-methyl-3-pyridyl]-3-methyl-4H-isoquinoline (0.500 g, 1.26 mmol) in MeOH (6 mL) and acetic acid (2 mL), sodium cyanoborohydride (0.079 g, 1.26 mmol) were added at 0° C. The reaction mixture was allowed to warm to room temperature and stirred overnight. The reaction mixture was diluted with AcOEt and water, quenched with Na₂CO₃ to pH 9. After phase separation, the aqueous phase extracted EtOAc. The combined organic phase were washed with aq. NaCl, dried over Na₂SO₄ and concentrated to give the tittle compound (0.471 g, 90%) as diasteromeric mixture (yellow oil).

¹H-NMR (CDCl₃, δ in ppm): 1.2-1.3 (s, 3H_{M}+3Hₘ), 2.3-2.6 (m, 3H_{M}+3Hₘ+2H_{M}+2Hₘ), 2.75 (d, 1H_{M}), 2.95 (d, 1H_{M}), 2.9-3.1 (s+s, 1Hₘ+1Hₘ), 5.1 (s, 1Hₘ), 5.2 (s, 1H_{M}), 6.1 (t, 1Hₘ), 6.2 (t, 1H_{M}), 6.6 (d, 1Hₘ), 6.5-6.9 (overlap t, 1H_{M}+1Hₘ), 7.1-7.3 (m, 4H_{M}, 4Hₘ), 7.55 (s, 1H_{M}+1Hₘ), 8.55 (s, 1H_{M}+1Hₘ).

*HPLC-MS: Rt=0.929 min; M⁺+H=397.1 Table I:

The positions of the heteroaryls given as “R⁷+R⁸” marked with “#” represents the connection points (carbon atoms 5′ and 6′ in formula I) with the remaining skeleton of the compounds of formula

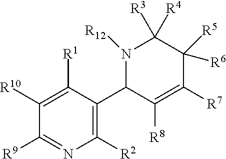

| | | | | | | | | | Mp [°C.]; HPLC-MS |
|---|---|---|---|---|---|---|---|---|---|
| | | | | | | | | | (Rₜ [min], M⁺ + H); |
| No. | R¹ | R² | R³ | R⁴ | R⁷ + R⁸ | R⁹ | R¹⁰ | R¹² | ¹H-NMR (δ in ppm) |
| I-1 | H | H | CH₃ | 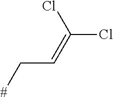 | 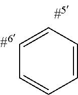 | CHF₂ | CH₃ | H | 0.929 min; M⁺ + H = 397.1 |
| I-2 | H | H | CF₃ | CF₃ | 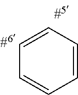 | CHF₂ | CH₃ | H | 1.30 min; M⁺ + H = 411.0 |
| I-3 | H | H | CH₃ | 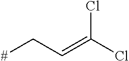 | 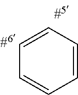 | CHF₂ | CH₃ | CH₃ | Isomerengemisch RT 1.025, M⁺ + H = 411.0; RT 1.077, M⁺ + H = 411.0; |
| I-4 | H | H | CH₃ | 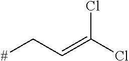 | 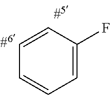 | CHF₂ | CH₃ | H | RT 0.969. M⁺ + H 415.0 |
| I-5 | H | H | CH₃ | 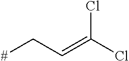 | 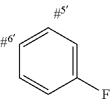 | CHF₂ | CH₃ | H | RT 0.958, M⁺ + H = 415.1 |
| I-6 | H | H | CH₃ | 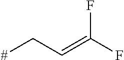 | 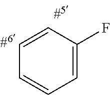 | CHF₂ | CH₃ | H | RT 0.901, M⁺ + H = 383.1 |
| I-7 | H | H | CH₃ | 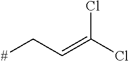 | 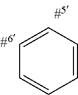 | CHF₂ | CH₃ | CO—CH₃ | RT 1.292, M⁺ + H = 439.1 |
| I-8 | H | H | CH₃ | 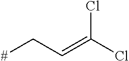 | 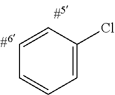 | CHF₂ | OCH₃ | H | RT 1.002, M⁺ + H = 448.9 |
| I-9 | H | H | CH₃ | 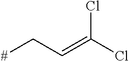 | 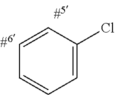 | CHF₂ | CH₃ | H | RT 1.020, M⁺ + H = 433.0 |
| I-10 | H | H | CH₃ | 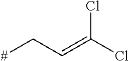 | 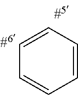 | CHF₂ | OCH₃ | H | RT 0.937, M⁺ + H = 413.0 |
| I-11 | H | H | CH₃ | 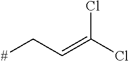 | 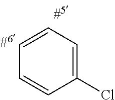 | CHF₂ | OCH₃ | H | RT 1.012, M⁺ + H = 447.0 |
| I-12 | H | H | CH₃ | 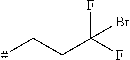 | 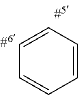 | CHF₂ | CH₃ | H | Isomerengemisch RT 0.942, M⁺ + H = 447.0; RT 0.960, M⁺ + H = 447.0; |
| I-13 | H | H | CH₃ | 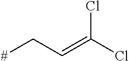 | 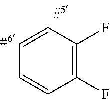 | CHF₂ | OCH₃ | H | RT 0.997, M⁺ + H = 449.0 |
| I-14 | H | H | CH₃ | 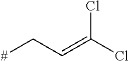 | 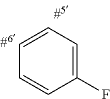 | CHF₂ | OCH₃ | H | RT 0.954, M⁺ + H = 431.1 |
| I-15 | H | H | CH₃ | CF₃ | 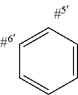 | CHF₂ | CH₃ | H | RT 1.250, M⁺ + H = 357.1 |
| I-16 | H | H | CH₃ | 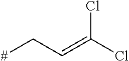 | 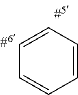 | CH₃OCO | CH₃ | H | RT 0.919, M⁺ + H = 405.0 |
| I-17 | H | H | CH₃ | 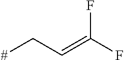 | 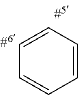 | CHF₂ | CH₃ | H | RT 0.932, M⁺ + H = 365.0 |

### II. Biological Trials

### Microtest

The active compounds were formulated separately as a stock solution having a concentration of 10000 ppm in dimethyl sulfoxide.

### Example 1—Activity Against the Grey Mold Botrytis cinerea in the Microtiterplate Test

The stock solutions were mixed according to the ratio, pipetted onto a micro titer plate (MTP) and diluted with water to the stated concentrations. A spore suspension of Botrci cinerea in a DOB medium solution was then added. The plates were placed in a water vapor-saturated chamber at a temperature of 18° C. Using an absorption photometer, the MTPs were measured at 405 nm 9 days after the inoculation.

In this test, the samples which had been treated with 31 ppm of the active substance from examples I-3, I-9, I-11 and I-12 respectively, showed up to at most 4% growth of the pathogen.

### Example 2—Activity Against Rice Blast Pyricularia oryzae in the Microtiterplate Test

The stock solutions were mixed according to the ratio, pipetted onto a micro titer plate (MTP) and diluted with water to the stated concentrations. A spore suspension of Pyricularia oryzae in a DOB medium solution was then added. The plates were placed in a water vapor-saturated chamber at a temperature of 18° C. Using an absorption photometer, the MTPs were measured at 405 nm 9 days after the inoculation.

In this test, the samples which had been treated with 31 ppm of the active substance from I-3, I-4, I-5, I-8, I-9, and I-12 respectively, showed up to at most 6% growth of the pathogen.

The measured parameters were compared to the growth of the active compound-free control variant (100%) and the fungus-free and active compound-free blank value to determine the relative growth in % of the pathogens in the respective active compounds.

Green House

The Spray Solutions were Prepared in Several Steps:

The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 5 ml.

Water was then added to total volume of 100 ml.

This stock solution was diluted with the described solvent-emulsifier-water mixture to the given concentration.

### Example 1—Preventative Fungicidal Control of Botrytis cinerea on Leaves of Green Pepper

Young seedlings of green pepper were grown in pots to the 4 to 5 leaf stage. These plants were sprayed to run-off with an aqueous suspension, containing the concentration of active ingredient or their mixture mentioned in the table below. The next day the plants were inoculated with an aqueous biomalt solution containing the spore suspension of Botrytis cinerea. Then the plants were immediately transferred to a humid chamber. After 5 days at 22 to 24° C. and a relative humidity close to 100% the extent of fungal attack on the leaves was visually assessed as % diseased leaf area.

In this test, the samples which had been treated with 250 ppm of the active substance from examples from I-1, I-2, I-3, I-4, I-5, I-6, I-7, I-8 and I-9 respectively, showed up to at most 4% growth of the pathogen whereas the untreated plants were 80% infected.

### Example 2—Long Lasting Control of Botrytis cinerea on Leaves of Green Pepper

Young seedlings of green pepper were grown in pots to the 4 to 5 leaf stage. These plants were sprayed to run-off with an aqueous suspension, containing the concentration of active ingredient or their mixture mentioned in the table below. The plants were then cultivated in the greenhouse for 7 days and then inoculated with an aqueous biomalt solution containing the spore suspension of Botrytis cinerea. Then the plants were immediately transferred to a humid chamber. After 5 days at 22 to 24° C. and a relative humidity close to 100% the extent of fungal attack on the leaves was visually assessed as % diseased leaf area.

In this test, the samples which had been treated with 250 ppm of the active substance from examples I-1, I-2, I-3, I-4, I-5, I-6, I-7, I-8 and I-9 respectively, showed up to at most 7% growth of the pathogen whereas the untreated plants were 90% infected.

1-16: (canceled)

17: A compound of formula I, or an N-oxide or an agriculturally acceptable salt thereof, 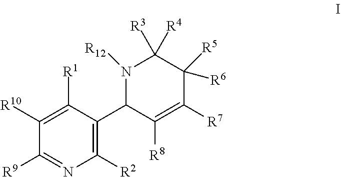
wherein
R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R¹ are unsubstituted or substituted with identical or different groups R^{1a} which independently of one another are selected from:
R^{1a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{1b} which independently of one another are selected from:
R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R² are unsubstituted or substituted with identical or different groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S); wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and
wherein R^{x}, R′, R″ and R″ are as defined above wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2 or
R³, R⁴ together with the carbon atom to which they are bound form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)n-C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
and
wherein the aliphatic moieties of R⁷⁸ are not further substituted or carry 1, 2, 3 or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R⁷⁸ are unsubstituted or substituted with identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R⁹ are unsubstituted or substituted by groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁹ are unsubstituted or substituted by groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R¹¹ are unsubstituted or carry 1, 2, 3 or up to the maximum possible number of identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R¹² are not further substituted or carry one, two, three or up to the maximum possible number of identical or different groups R^{12a} which independently of one another are selected from:
R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
with the proviso that if
R⁷, R⁸ together with the carbon atoms to which they are bound form a ring A, wherein the ring A is phenyl
R¹ is hydrogen; and
R⁴ cannot be an unsubstituted C₁-C₆-alkyl.

18: The compound of claim 17, wherein
R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R¹ are unsubstituted or substituted with identical or different groups R^{1a} which independently of one another are selected from:
R^{1a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{1b} which independently of one another are selected from:
R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R² are unsubstituted or substituted with identical or different groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2 or
R³, R⁴ together with the carbon atom to which they are bound form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)n-C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
and
wherein the aliphatic moieties of R⁷⁸ are not further substituted or carry 1, 2, 3 or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R⁷⁸ are unsubstituted or substituted with identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R⁹ are unsubstituted or substituted by groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁹ are unsubstituted or substituted by groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R¹¹ are unsubstituted or carry 1, 2, 3 or up to the maximum possible number of identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R¹² are not further substituted or carry one, two, three or up to the maximum possible number of identical or different groups R^{12a} which independently of one another are selected from:
R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
and the N-oxides and the agriculturally acceptable salts thereof.

19: The compound of claim 17, wherein
R¹ is H;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R² are unsubstituted or substituted with identical or different groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S); wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and
wherein R^{x}, R′, R″ and R″ are as defined above wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2 or
R³, R⁴ together with the carbon atom to which they are bound form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)n-C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
and
wherein the aliphatic moieties of R⁷⁸ are not further substituted or carry 1, 2, 3 or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R⁷⁸ are unsubstituted or substituted with identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R⁹ are unsubstituted or substituted by groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁹ are unsubstituted or substituted by groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R¹¹ are unsubstituted or carry 1, 2, 3 or up to the maximum possible number of identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R¹² are not further substituted or carry one, two, three or up to the maximum possible number of identical or different groups R^{12a} which independently of one another are selected from:
R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
and the N-oxides and the agriculturally acceptable salts thereof.

20: The compound of claim 17, wherein R² is H, F, Cl, Br, CN, C₁-C₆-alkyl, C₂-C₆-alkenyl, C₂-C₆-alkynyl, C₁-C₆-alkoxy, C₃-C₆-cycloalkyl, wherein the acyclic moieties of R² are unsubstituted or substituted by halogen.

21: The compound of claim 17, wherein R⁴ is independently selected from CN, C₁-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl, a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂-Rx, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl.

22: The compound of claim 17, wherein R⁴ is independently selected from CN, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl.

23: The compound of claim 17, wherein R⁷ and R⁸ together with the carbon atoms to which they are bound form a phenyl, wherein the phenyl carries zero, one, two, three or four substituents (R⁷⁸)ₒ.

24: The compound of claim 17, wherein R⁷ and R⁸ together with the carbon atoms to which they are bound form a five-membered heteroaryl; wherein the heteroaryl contains one, two or three heteroatoms selected from N, O and S, and wherein the heteroaryl carries zero, one, two, three or four substituents (R⁷⁸)ₒ.

25: The compound of claim 17, wherein R⁷⁸ is in each case hydrogen, halogen, C₁-C₆-alkyl, C₁-C₆-halogenalkyl, C₁-C₆-alkoxy, C₁-C₆-halogenalkoxy or CN.

26: The compound of claim 17, wherein R⁹ and R¹⁰ independently are selected from CN, halogen, C₁-C₆-alkyl, C₁-C₆-halogenalkyl, C₂-C₆-alkynyl, OR^{Y}, C₃-C₆-cycloalkyl.
R^{Y} is C₁-C₆-alkyl, C₁-C₆-halogenalkyl, C₂-C₆-alkenyl or C₂-C₆-alkynyl.

27: The compound of claim 17, wherein R¹² is hydrogen, C(═O)C₁-C₆-alkyl, C(═O)OC₁-C₆-alkyl, C(═O)NHC₁-C₆-alkyl, S(O)₂—C₁-C₆-alkyl, S(O)₂-aryl, SO₂—NH(C₁-C₆-alkyl), OR^{Y}, or C₁-C₄-alkyl.
R^{Y} is C₁-C₆-alkyl, C₁-C₆-halogenalkyl, C₂-C₆-alkenyl or C₂-C₆-alkynyl.

28: A composition comprising one compound of formula I, as defined in claim 17, an N-oxide or an agriculturally acceptable salt thereof.

29: A method for combating phytopathogenic fungi, comprising treating the fungi or the materials, plants, the soil or seeds to be protected against fungal attack with an effective amount of at least one compound of formula I, as defined in claim 17.

30: Seed coated with at least one compound of the formula I, as defined in claim 17, or an agriculturally acceptable salt thereof in an amount of from 0.1 to 10 kg per 100 kg of seed.

31: An intermediate of formula XV 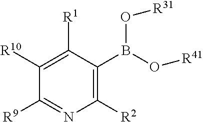
wherein R¹ is hydrogen, R² is hydrogen, R⁹ is CH₂F or CHF₂, R¹⁰ is C₁-C₆-alkyl, F, Cl, Br or C₁-C₆-alkoxy, R³¹ is H or CH₃, R⁴¹ is H or CH₃ or R³¹ and R⁴¹ together form a tetramethyl-1,3,2-dioxaborolan-ring.

32: The method of claim 29, wherein
R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R¹ are unsubstituted or substituted with identical or different groups R^{1a} which independently of one another are selected from:
R^{1a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{1b} which independently of one another are selected from:
R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R² are unsubstituted or substituted with identical or different groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2 or
R³, R⁴ together with the carbon atom to which they are bound form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)n-C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
and
wherein the aliphatic moieties of R⁷⁸ are not further substituted or carry 1, 2, 3 or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R⁷⁸ are unsubstituted or substituted with identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R⁹ are unsubstituted or substituted by groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁹ are unsubstituted or substituted by groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R¹¹ are unsubstituted or carry 1, 2, 3 or up to the maximum possible number of identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R¹² are not further substituted or carry one, two, three or up to the maximum possible number of identical or different groups R^{12a} which independently of one another are selected from:
R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
and the N-oxides and the agriculturally acceptable salts thereof.

33: The method of claim 29, wherein
R¹ is H;
R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
wherein the aliphatic moieties of R² are unsubstituted or substituted with identical or different groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R¹ are not further substituted or carry 1, 2, 3, 4, 5 or up to the maximum number of identical or different groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R³ is in each case independently selected from CH₃, CH₂F, CHF₂ and CF₃;
R⁴ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of R⁴ are independently not further substituted or carry 1, 2, 3 or up to the maximum possible number of identical or different groups R^{4a}, respectively, which independently of one another are selected from:
R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S); wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁴ are independently not further substituted or carry 1, 2, 3, 4, 5 or up to the maximum number of identical or different groups R^{4b}, respectively, which independently of one another are selected from:
R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2 or
R³, R⁴ together with the carbon atom to which they are bound form a saturated or partially unsaturated three-, four-, five-, six-, seven-, eight-, nine-, or ten-membered carbocycle or heterocycle; wherein the heterocycle contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted phenyl or phenyl that is substituted by 1, 2 or 3 substituents selected from CN, C₁-C₄-alkyl, halogen, C₁-C₄-halogenalkyl, C₁-C₄-alkoxy and C₁-C₄-halogenalkoxy; and wherein S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-halogenalkyl, C₁-C₆-alkoxy, C₁-C₆-halogenalkoxy, C₁-C₆-alkylthio, C₁-C₆-halogenalkylthio, C₁-C₄-alkoxy-C₁-C₄-alkyl, phenyl and phenoxy, wherein the phenyl groups are unsubstituted or substituted with substituents R^{34a} selected from the group consisting of CN, halogen, OH, C₁-C₄-alkyl, C₁-C₄-halogenalkyl, C₁-C₄-alkoxy and C₁-C₄-halogenalkoxy; and wherein in each case one or two CH₂ groups of the carbo- or heterocycle may be replaced by a group independently selected from C(═O) and C(═S);
R⁵, R⁶ is hydrogen,
R⁷, R⁸ together with the carbon atoms to which they are bound form a ring A, wherein the ring A is phenyl or five- or six-membered heteroaryl; wherein the heteroaryl contains one, two or three heteroatoms selected from N, O and S, and wherein the ring A is substituent by (R⁷⁸)ₒ, wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₂-C₆-alkenyloxy, C₂-C₆-alkynyloxy, C₃-C₆-cycloalkyl, C₃-C₆-cycloalkenyl, S(O)n-C₁-C₆-alkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein n, R′ and R″ is as defined above;
and
wherein the aliphatic moieties of R⁷⁸ are not further substituted or carry 1, 2, 3 or up to the maximum possible number of identical or different groups R^{78a} which independently of one another are selected from:
R^{78a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or unsubstituted or substituted with R^{78aa} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R⁷⁸ are unsubstituted or substituted with identical or different groups R^{78b} which independently of one another are selected from:
R^{78b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁹ is in each case independently selected from hydrogen, halogen, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), C(═O)N(C₁-C₆-alkyl)₂, C(═O)N(C₂-C₆-alkenyl)₂, C(═O)N(C₂-C₆-alkynyl)₂, C(═O)N(C₃-C₇-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R⁹ are unsubstituted or substituted by groups R^{9a} which independently of one another are selected from:
R^{9a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁹ are unsubstituted or substituted by groups R^{9b} which independently of one another are selected from:
R^{9b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above
R¹⁰ is in each case independently selected from the substituents as defined for R⁹, wherein the possible substituents for R¹⁰ are R^{10a} and R^{10b}, respectively, which correspond to R^{9a} and R^{9b}, respectively;
R⁹, R¹⁰ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R¹¹)ₘ, wherein m is 0, 1, 2, 3 or 4;
R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R¹¹ are unsubstituted or carry 1, 2, 3 or up to the maximum possible number of identical or different groups R^{11a} which independently of one another are selected from:
R^{11a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R¹¹ are unsubstituted or substituted with identical or different groups R^{11b} which independently of one another are selected from:
R^{11b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R¹² is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C(═O)O(C₁-C₆-alkyl), C(═O)O(C₂-C₆-alkenyl), C(═O)O(C₂-C₆-alkynyl), C(═O)O(C₃-C₆-cycloalkyl), C(═O)NH(C₁-C₆-alkyl), C(═O)NH(C₂-C₆-alkenyl), C(═O)NH(C₂-C₆-alkynyl), C(═O)NH(C₃-C₆-cycloalkyl), C(═O)N(C₁-C₆-alkyl)₂, C(═O)N(C₂-C₆-alkenyl)₂, C(═O)N(C₂-C₆-alkynyl)₂, C(═O)N(C₃-C₆-cycloalkyl)₂, CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₁-C₆-alkyl), C(═S)O(C₂-C₆-alkenyl), C(═S)O(C₂-C₆-alkynyl), C(═S)O(C₃-C₆-cycloalkyl), C(═S)NH(C₁-C₆-alkyl), C(═S)NH(C₂-C₆-alkenyl), C(═S)NH(C₂-C₆-alkynyl), C(═S)NH(C₃-C₆-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₆-cycloalkyl)₂, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₁-C₆-alkylthio, C₁-C₆-halogenalkylthio, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, S(O)ₙ—C₁-C₆-alkoxy, S(O)ₙ—C₂-C₆-alkenyl, S(O)ₙ—C₂-C₆-alkynyl, S(O)ₙaryl, SO₂—NH(C₁-C₆-alkyl), SO₂—NH(C₁-C₆-halogenalkyl), SO₂—NH-aryl, tri-(C₁-C₆ alkyl)silyl and di-(C₁-C₆ alkoxy)phosphoryl), five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R¹² are not further substituted or carry one, two, three or up to the maximum possible number of identical or different groups R^{12a} which independently of one another are selected from:
R^{12a} halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{12b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
and the N-oxides and the agriculturally acceptable salts thereof.

34: The method of claim 29, wherein R² is H, F, Cl, Br, CN, C₁-C₆-alkyl, C₂-C₆-alkenyl, C₂-C₆-alkynyl, C₁-C₆-alkoxy, C₃-C₆-cycloalkyl, wherein the acyclic moieties of R² are unsubstituted or substituted by halogen.

35: The method of claim 29, wherein R⁴ is independently selected from CN, C₁-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl, a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl.

36: The method of claim 29, wherein R⁴ is independently selected from CN, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl.

37: The method of claim 29, wherein R⁷ and R⁸ together with the carbon atoms to which they are bound form a phenyl, wherein the phenyl carries zero, one, two, three or four substituents (R⁷⁸)ₒ.

38: The method of claim 29, wherein R⁷ and R⁸ together with the carbon atoms to which they are bound form a five-membered heteroaryl; wherein the heteroaryl contains one, two or three heteroatoms selected from N, O and S, and wherein the heteroaryl carries zero, one, two, three or four substituents (R⁷⁸)ₒ.

39: The method of claim 29, wherein R⁷⁸ is in each case hydrogen, halogen, C₁-C₆-alkyl, C₁-C₆-halogenalkyl, C₁-C₆-alkoxy, C₁-C₆-halogenalkoxy or CN.

40: The method of claim 29, wherein R⁹ and R¹⁰ independently are selected from CN, halogen, C₁-C₆-alkyl, C₁-C₆-halogenalkyl, C₂-C₆-alkynyl, OR^{Y}, C₃-C₆-cycloalkyl.
R^{Y} is C₁-C₆-alkyl, C₁-C₆-halogenalkyl, C₂-C₆-alkenyl or C₂-C₆-alkynyl.

41: The method of claim 29, wherein R¹² is hydrogen, C(═O)C₁-C₆-alkyl, C(═O)OC₁-C₆-alkyl, C(═O)NHC₁-C₆-alkyl, S(O)₂—C₁-C₆-alkyl, S(O)₂-aryl, SO₂—NH(C₁-C₆-alkyl), OR^{Y}, or C₁-C₄-alkyl.
R^{Y} is C₁-C₆-alkyl, C₁-C₆-halogenalkyl, C₂-C₆-alkenyl or C₂-C₆-alkynyl.
